SEQUENCE LISTING (50 sequences)

SEQ ID NO: 1 - DNA, 20 bp, synthetic construct
  source 1..20: mol_type = other DNA
attgccggtcgctatcacat

SEQ ID NO: 2 - DNA, 20 bp, synthetic construct
  source 1..20: mol_type = other DNA
tgtccagagggagacaccaa

SEQ ID NO: 3 - protein, 7 aa, synthetic construct
PMPWRVI

SEQ ID NO: 4 - protein, 16 aa, synthetic construct
EAAAKEAAAKEAAAKA

SEQ ID NO: 5 - protein, 5 aa, synthetic construct
EAAAK

SEQ ID NO: 6 - protein, 65 aa, synthetic construct
PWRVICATSSQFTQITEHNSRRSANYQPNLWNFEFLQSLENDLKVEKLEEKATKLEEEVRPWRVI

SEQ ID NO: 7 - protein, 6 aa, synthetic construct
HHHHHH

SEQ ID NO: 8 - DNA, 20 bp, synthetic construct
  source 1..20: mol_type = other DNA
tgacatggaaatcatcctcc

SEQ ID NO: 9 - DNA, 23 bp, synthetic construct
  source 1..23: mol_type = other DNA
ggtggaaacggcttcagttaaag

SEQ ID NO: 10 - DNA, 24 bp, synthetic construct
  source 1..24: mol_type = other DNA
tgaggaattaggaggtaatatatg

SEQ ID NO: 11 - DNA, 24 bp, synthetic construct
  source 1..24: mol_type = other DNA
gccttgtaaatacaaattatcatg

SEQ ID NO: 12 - DNA, 2376 bp, synthetic construct
  source 1..2376: mol_type = other DNA
atgttcgacgtattcactcgggttgtttcccaagctgatgctcgcggcgagtacctctctggttctcagttagatgctttgagcgctaccgttgctgaaggcaacaaacggattgattctgttaaccgcatcaccggtaatgcttccgctatcgtttccaacgctgctcgtgctttgttcgccgaacagccccaattaatccaacccggtggaaacgcctacaccagccgtcgtatggctgcttgtttgcgtgacatggaaatcatcctccgctatgttacctacgcaaccttcaccggcgacgcttccgttctagaagatcgttgcttgaacggtctccgtgaaacctacgttgccctgggtgttcccggtgcttccgtagctgctggcgttcaaaaaatgaaagaagctgccctggacatcgttaacgatcccaatggcatcacccgtggtgattgcagtgctatcgttgctgaaatcgctggttacttcgaccgcgccgctgctgccgtagcccaccatcaccatcaccatgataatttgtatttacaaggctgtgacttgcctcagacgcattctttgggaagccgacgcacactgatgctgctcgcccaaatgcgccggatctccttattctcctgtctcaaggatcggcatgacttcggcttccctcaggaggagtttggaaatcagttccaaaaggccgaaaccattccggtcctccatgaaatgattcaacagatctttaacttattcagtaccaaagacagcagtgcggcctgggacgaaacattactcgataaattctacacggaattataccaacagttgaacgacttagaagcctgtgtaatccaaggtgttggtgtcactgagactccattaatgaaagaagactctattctggccgtccgcaagtatttccagcgaatcacactgtatttgaaagagaaaaagtattctccgtgtgcgtgggaggtagtacgggctgaaatcatgcggtccttctctttaagcacaaacctccaggaatctctgcgctccaaagaatgaagatctgcggccgcgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggatcctctggttattttaaaaaccaactttactcaggttccatacccgagaaaatccagcttaaagctgacatatctaggaaaattttcacattctaacgggagataccagaacaatgaaaacccctttaactgaagccgtttccaccgctgactctcaaggtcgctttctgagcagcaccgaattgcaaattgctttcggtcgtctacgtcaagctaatgctggtttgcaagccgctaaagctctgaccgacaatgcccagagcttggtaaatggtgctgcccaagccgtttataacaaattcccctacaccacccaaacccaaggcaacaactttgctgcggatcaacggggtaaagacaagtgtgcccgggacatcggctactacctccgcatcgttacctactgcttagttgctggtggtaccggtcctttggatgagtacttgatcgccggtattgatgaaatcaaccgcacctttgacctctcccccagctggtatgtt

SEQ ID NO: 13 - protein, 343 aa, synthetic construct
  VARIANT 172: note = Any amino acid
MFDVFTRVVSQADARGEYLSGSQLDALSATVAEGNKRIDSVNRITGNASAIVSNAARALFAEQPQLIQPGGNAYTSRRMAACLRDMEIILRYVTYATFTGDASVLEDRCLNGLRETYVALGVPGASVAAGVQKMKEAALDIVNDPNGITRGDCSAIVAEIAGYFDRAAAAVXHHHHHHENLYFQGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQ

SEQ ID NO: 14 - DNA, 3243 bp, synthetic construct
  source 1..3243: mol_type = other DNA
atgttcgacgtattcactcgggttgtttcccaagctgatgctcgcggcgagtacctctctggttctcagttagatgctttgagcgctaccgttgctgaaggcaacaaacggattgattctgttaaccgcatcaccggtaatgcttccgctatcgtttccaacgctgctcgtgctttgttcgccgaacagccccaattaatccaacccggtggaaacgcctacaccagccgtcgtatggctgcttgtttgcgtgacatggaaatcatcctccgctatgttacctacgcaaccttcaccggcgacgcttccgttctagaagatcgttgcttgaacggtctccgtgaaacctacgttgccctgggtgttcccggtgcttccgtagctgctggcgttcaaaaaatgaaagaagctgccctggacatcgttaacgatcccaatggcatcacccgtggtgattgcagtgctatcgttgctgaaatcgctggttacttcgaccgcgccgctgctgccgtagcccccatgccttggcgcgtgattcaccatcaccatcaccatgataatttgtatttacaaggcaagaacttagactgttgggtcgataatgaggaggatatcgatgtcattctaaagaagtctaccatcctaaatctggacattaacaatgatatcattagtgatatttctggttttaattcttctgttatcacataccccgacgcccaattagttccaggaattaatgggaaggctattcatctagtaaataatgagagcagcgaagtgatcgtccacaaggcgatggacattgagtataatgatatgttcaacaactttactgtgtccttttggttgcgcgtccccaaagtgtctgccagtcacctggaacaatacgacacgaatgaatatagtatcattagcagtatgaaaaagtatagtttaagtattgggtctgggtggtccgtctctctcaaaggaaacaacctcatctggaccctcaaggattctgcaggcgaagtgcgtcaaattacattccgcgacttgtccgataaattcaatgcgtacctcgctaacaaatgggttttcatcaccatcacgaacgaccggctgagtagcgctaacctctacattaatggcgtgttgatggggagtgcggagatcaccggcctgggggcaattcgcgaggacaacaacatcacactcaagttggaccgttgcaataacaacaaccaatatgtctctatcgacaaatttcgtattttctgtaaggcgctaaacccaaaggagatcgaaaagttatatactagttatttgagcatcacgtttttacgcgatttttggggcaacccactgcgttatgacactgaatattatctcattcccgttgcgtacagcagtaaagacgtccaattaaagaatatcacggattatatgtatctgactaatgctcccagttacacgaacgggaaattaaacatttactaccgccgtctgtactctggtctgaagtttattatcaaacgctacacccccaacaatgaaatcgactcttttgttcggtctggtgactttattaaactgtacgtaagttacaacaacaatgaacacatcgtgggataccctaaagacgggaatgcgttcaataacttagatcggatcctccgagtagggtataatgcacccggtattcctctgtataagaagatggaagcggtaaagctccgtgacctcaaaacttatagcgtgcaactcaaactgtacgacgacaaagatgcgtctctagggttggtgggtacccacaacggacaaatcgggaatgaccctaaccgcgatattctaatcgcttctaattggtattttaaccacttaaaagataagaccctcacctgcgactggtatttcgtcccaaccgacgagggatggactaatgattgaggaattaggaggtaatatatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatttttttaaggcagttattggtgcccttaaacgcctggggatcctctggttattttaaaaaccaactttactcaggttccatacccgagaaaatccagcttaaagctgacatatctaggaaaattttcacattctaacgggagataccagaacaatgaaaacccctttaactgaagccgtttccaccgctgactctcaaggtcgctttctgagcagcaccgaattgcaaattgctttcggtcgtctacgtcaagctaatgctggtttgcaagccgctaaagctctgaccgacaatgcccagagcttggtaaatggtgctgcccaagccgtttataacaaattcccctacaccacccaaacccaaggcaacaactttgctgcggatcaacggggtaaagacaagtgtgcccgggacatcggctactacctccgcatcgttacctactgcttagttgctggtggtaccggtcctttggatgagtacttgatcgccggtattga

SEQ ID NO: 15 - protein, 643 aa, synthetic construct
  VARIANT 172: note = Any amino acid
MFDVFTRVVSQADARGEYLSGSQLDALSATVAEGNKRIDSVNRITGNASAIVSNAARALFAEQPQLIQPGGNAYTSRRMAACLRDMEIILRYVTYATFTGDASVLEDRCLNGLRETYVALGVPGASVAAGVQKMKEAALDIVNDPNGITRGDCSAIVAEIAGYFDRAAAAVXPMPWRVIHHHHHHENLYFQGKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYDTNEYSIISSMKKYSLSIGSGWSVSLKGNNLIWTLKDSAGEVRQITFRDLSDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVAYSSKDVQLKNITDYMYLTNAPSYTNGKLNIYYRRLYSGLKFIIKRYTPNNEIDSFVRSGDFIKLYVSYNNNEHIVGYPKDGNAFNNLDRILRVGYNAPGIPLYKKMEAVKLRDLKTYSVQLKLYDDKDASLGLVGTHNGQIGNDPNRDILIASNWYFNHLKDKTLTCDWYFVPTDEGWTND

SEQ ID NO: 16 - DNA, 1890 bp, synthetic construct
  source 1..1890: mol_type = other DNA
atgttcgacgtattcactcgggttgtttcccaagctgatgctcgcggcgagtacctctctggttctcagttagatgctttgagcgctaccgttgctgaaggcaacaaacggattgattctgttaaccgcatcaccggtaatgcttccgctatcgtttccaacgctgctcgtgctttgttcgccgaacagccccaattaatccaacccggtggaaacgcctacaccagccgtcgtatggctgcttgtttgcgtgacatggaaatcatcctccgctatgttacctacgcaaccttcaccggcgacgcttccgttctagaagatcgttgcttgaacggtctccgtgaaacctacgttgccctgggtgttcccggtgcttccgtagctgctggcgttcaaaaaatgaaagaagctgccctggacatcgttaacgatcccaatggcatcacccgtggtgattgcagtgctatcgttgctgaaatcgctggttacttcgaccgcgccgctgctgccgtagcccccatgccttggcgcgtgattcaccatcaccatcaccatgataatttgtatttacaaggctgaggaattaggaggtaatatatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatttttttaaggcagttattggtgcccttaaacgcctggggatcctctggttattttaaaaaccaactttactcaggttccatacccgagaaaatccagcttaaagctgacatatctaggaaaattttcacattctaacgggagataccagaacaatgaaaacccctttaactgaagccgtttccaccgctgactctcaaggtcgctttctgagcagcaccgaattgcaaattgctttcggtcgtctacgtcaagctaatgctggtttgcaagccgctaaagctctgaccgacaatgcccagagcttggtaaatggtgctgcccaagccgtttataacaaattcccctacaccacccaaacccaaggcaacaactttgctgcggatcaacggggtaaagacaagtgtgcccgggacatcggctactacctccgcatcgttacctactgcttagttgctggtggtaccggtcctttggatgagtacttgatcgccggtattga

SEQ ID NO: 17 - protein, 192 aa, synthetic construct
  VARIANT 172: note = Any amino acid
MFDVFTRVVSQADARGEYLSGSQLDALSATVAEGNKRIDSVNRITGNASAIVSNAARALFAEQPQLIQPGGNAYTSRRMAACLRDMEIILRYVTYATFTGDASVLEDRCLNGLRETYVALGVPGASVAAGVQKMKEAALDIVNDPNGITRGDCSAIVAEIAGYFDRAAAAVXPMPWRVIHHHHHHENLYFQG

SEQ ID NO: 18 - DNA, 2309 bp, synthetic construct
  source 1..2309: mol_type = other DNA
atgaaaacccctttaactgaagccgtttccaccgctgactctcaaggtcgctttctgagcagcaccgaattgcaaattgctttcggtcgtctacgtcaagctaatgctggtttgcaagccgctaaagctctgaccgacaatgcccagagcttggtaaatggtgctgcccaagccgtttataacaaattcccctacaccacccaaacccaaggcaacaactttgctgcggatcaacggggtaaagacaagtgtgcccgggacatcggctactacctccgcatcgttacctactgcttagttgctggtggtaccggtcctttggatgagtacttgatcgccggtattgatgaaatcaaccgcacctttgacctctcccccagctggtatgttgaagctctgaaatacatcaaagctaaccacggcttgagtggcgatgcccgtgacgaagctaattcctacctcgattacgccatcaatgctctgagccccatgccttggcgcgtgattcaccatcaccatcaccatgaaaatttgtatttacaaggctgtgacttgcctcagacgcattctttgggaagccgacgcacactgatgctgctcgcccaaatgcgccggatctccttattctcctgtctcaaggatcggcatgacttcggcttccctcaggaggagtttggaaatcagttccaaaaggccgaaaccattccggtcctccatgaaatgattcaacagatctttaacttattcagtaccaaagacagcagtgcggcctgggacgaaacattactcgataaattctacacggaattataccaacagttgaacgacttagaagcctgtgtaatccaaggtgttggtgtcactgagactccattaatgaaagaagactctattctggccgtccgcaagtatttccagcgaatcacactgtatttgaaagagaaaaagtattctccgtgtgcgtgggaggtagtacgggctgaaatcatgcggtccttctctttaagcacaaacctccaggaatctctgcgctccaaagaatgagcggccgcgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatcagtttttaattctagctggcctggaagggtgggaaagtgttaacaactggttgacaatattccgcttttccaggtcttgtcgtatttattgacacttatctaggagaacaaaatgactagtttagtttcggcccagcgtctgggcattgtggccgtggatgaggctattcccctcgagcttcgttcccggagtacagaggaagaggtggatgccgttatcctggcggtttaccgtcaagttttgggcaacgatcatctcatgtcccaggaacgactgaccagtgcagaatctttgctccggggcagggagatttcggtaagggattttgttcgggctgtggctctgtcggaagtgtaccggcagaagtttttccattccaacccacaaaatcgttttatcgagcttaattataagcatttactgggacgggctccctacgatcagtcggaaattgctttccacaccgatctttatcaccaggggggctat

SEQ ID NO: 19 - protein, 162 aa, synthetic construct
MKTPLTEAVSTADSQGRFLSSTELQIAFGRLRQANAGLQAAKALTDNAQSLVNGAAQAVYNKFPYTTQTQGNNFAADQRGKDKCARDIGYYLRIVTYCLVAGGTGPLDEYLIAGIDEINRTFDLSPSWYVEALKYIKANHGLSGDARDEANSYLDYAINALS

SEQ ID NO: 20 - protein, 7 aa, synthetic construct
ENLYLQG

SEQ ID NO: 21 - protein, 165 aa, synthetic construct [sequence omitted]

SEQ ID NO: 22 - protein, 7 aa, synthetic construct
AAALIGT

SEQ ID NO: 23 - protein, 9 aa, synthetic construct
EVPTFTIMK

SEQ ID NO: 24 - protein, 19 aa, synthetic construct
DHYRAYFLSYRDFQELRKL

SEQ ID NO: 25 - protein, 219 aa, synthetic construct
MEKKITGYTTVDISQWHRKEHFEAFQSVAQCTYNQTVQLDITAFLKTVKKNKHKFYPAFIHILARLMNAHPEFRMAMKDGELVIWDSVHPCYTVFHEQTETFSSLWSEYHDDFRQFLHIYSQDVACYGENLAYFPKGFIENMFFVSANPWVSFTSFDLNVANMDNFFAPVFTMGKYYTQGDKVLMPLAIQVHHAVCDGFHVGRMLNELQQYCDEWQGGA

SEQ ID NO: 26 - protein, 14 aa, synthetic construct
SVFNSSWPGRVGKC

SEQ ID NO: 27 - protein, 19 aa, synthetic construct
QLVDNIPLFQVLSYLLTLI

SEQ ID NO: 28 - protein, 126 aa, synthetic construct
MTSLVSAQRLGIVAVDEAIPLELRSRSTEEEVDAVILAVYRQVLGNDHLMSQERLTSAESLLRGREISVRDFVRAVALSEVYRQKFFHSNPQNRFIELNYKHLLGRAPYDQSEIAFHTDLYHQGGY

SEQ ID NO: 29 - DNA, 2578 bp, synthetic construct
  source 1..2578: mol_type = other DNA
atggctcttcccctattgaactacgcccccaaaagtcaaaatgtgagggtagaaggttatgaaattggctctgaagagaagcctgttgttttcaccacggaaaacatcctctccagcagcgatatggataacctaatcgaggcggcctatcgtcaaatctttttccatgcgtttaagtgggaccgagaaaaagtccttgagtcccaactgcgtaacggccaaattactgtacgagattttgtgcggggcttgttgctttccaacaccttccgcaatagcttctacgaaaagaatagtaactaccgcttcgttgagcactgtgtacagaagattttagggcgggacgtttacagcgaacgggaaaaaattgcttggtccattgtggtcgcgaccaagggctatcaaggattaattgacgatttgctcaacagcgacgagtacctcaataactttggctatgacacggtgccctaccaacgtcgtcgcaaccttcccggtcgggaagcgggtgaattgccctttaacatcaaatctccccggtacgatgcctaccaccgtcgccaattgggcttcccccaaatcgtttggcagaacgaagtgcgtcgctttattccccaggagaaaaagctcaccgctggcaatccgatgaacttcttgggcatggcccgcagtatcaaccctgccgccaacaccattcccaaggtttccgcccaaaatatcaacatcgaagcttctgtgccccgtcgccccatgccttggcgcgtgattcaccatcaccatcaccatgaaaatttgtatttacaaggctgtgacttgcctcagacgcattctttgggaagccgacgcacactgatgctgctcgcccaaatgcgccggatctccttattctcctgtctcaaggatcggcatgacttcggcttccctcaggaggagtttggaaatcagttccaaaaggccgaaaccattccggtcctccatgaaatgattcaacagatctttaacttattcagtaccaaagacagcagtgcggcctgggacgaaacattactcgataaattctacacggaattataccaacagttgaacgacttagaagcctgtgtaatccaaggtgttggtgtcactgagactccattaatgaaagaagactctattctggccgtccgcaagtatttccagcgaatcacactgtatttgaaagagaaaaagtattctccgtgtgcgtgggaggtagtacgggctgaaatcatgcggtccttctctttaagcacaaacctccaggaatctctgcgctccaaagaatgagcggccgcgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaagcactaaggtcagagggttgaaggattagtgcattcctagcgtggatagttactgatttttccgtcctgaaaattaaactgaaaaatcaaataattgtttcgctgggactgtttgctagtcccttttttttggctatttttggggcgaggatctgagtataaatacttgattgaactatgacgggagagcaatttagcggaaattgtgaagtcttctaacaattatggaacctctgcccataatcccagactaaaaacctatacttgcccccaataccaactcctcaccgaacctagggcttgatgttggttgaggtagtaatttccccagagtggagtccccatgattgcccctatttgccgtgctctgcgttcccgtcttcccctcgccgctatgctgatgattgccgccactgctacccccgccttagcgaattcccccctcgccgccaatttaactccgaatcaaccccaacctttactgttggatggcgttcaac

SEQ ID NO: 30 - protein, 249 aa, synthetic construct
MALPLLNYAPKSQNVRVEGYEIGSEEKPVVFTTENILSSSDMDNLIEAAYRQIFFHAFKWDREKVLESQLRNGQITVRDFVRGLLLSNTFRNSFYEKNSNYRFVEHCVQKILGRDVYSEREKIAWSIVVATKGYQGLIDDLLNSDEYLNNFGYDTVPYQRRRNLPGREAGELPFNIKSPRYDAYHRRQLGFPQIVWQNEVRRFIPQEKKLTAGNPMNFLGMARSINPAANTIPKVSAQNINIEASVPRR

SEQ ID NO: 31 - protein, 6 aa, synthetic construct
ALRSEG

SEQ ID NO: 32 - protein, 44 aa, synthetic construct
RISAFLAWIVTDFSVLKIKLKNQIIVSLGLFASPFFLAIFGARI

SEQ ID NO: 33 - protein, 14 aa, synthetic construct
TMTGEQFSGNCEVF

SEQ ID NO: 34 - protein, 7 aa, synthetic construct
QLWNLCP

SEQ ID NO: 35 - protein, 17 aa, synthetic construct
SQTKNLYLPPIPTPHRT

SEQ ID NO: 36 - protein, 66 aa, synthetic construct
GLMLVEVVISPEWSPHDCPYLPCSAFPSSPRRYADDCRHCYPRLSEFPPRRQFNSESTPTFTVGWR

SEQ ID NO: 37 - DNA, 2440 bp, synthetic construct
  source 1..2440: mol_type = other DNA
atgttcgacgtattcactcgggttgtttcccaagctgatgctcgcggcgagtacctctctggttctcagttagatgctttgagcgctaccgttgctgaaggcaacaaacggattgattctgttaaccgcatcaccggtaatgcttccgctatcgtttccaacgctgctcgtgctttgttcgccgaacagccccaattaatccaacccggtggaaacgcctacaccagccgtcgtatggctgcttgtttgcgtgacatggaaatcatcctccgctatgttacctacgcaaccttcaccggcgacgcttccgttctagaagatcgttgcttgaacggtctccgtgaaacctacgttgccctgggtgttcccggtgcttccgtagctgctggcgttcaaaaaatgaaagaagctgccctggacatcgttaacgatcccaatggcatcacccgtggtgattgcagtgctatcgttgctgaaatcgctggttacttcgaccgcgccgctgctgccgtagcctagtctggttattttaaaaaccaactttactcaggttccatacccgagaaaatccagcttaaagctgacatatctaggaaaattttcacattctaacgggagataccagaacaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaggttattttaaaaaccaactttactcaggttccatacccgagaaaatccagcttaaagctgacatatctaggaaaattttcacattctaacgggagataccagaacaatgtgtgacttgcctcagacgcattctttgggaagccgacgcacactgatgctgctcgcccaaatgcgccggatctccttattctcctgtctcaaggatcggcatgacttcggcttccctcaggaggagtttggaaatcagttccaaaaggccgaaaccattccggtcctccatgaaatgattcaacagatctttaacttattcagtaccaaagacagcagtgcggcctgggacgaaacattactcgataaattctacacggaattataccaacagttgaacgacttagaagcctgtgtaatccaaggtgttggtgtcactgagactccattaatgaaagaagactctattctggccgtccgcaagtatttccagcgaatcacactgtatttgaaagagaaaaagtattctccgtgtgcgtgggaggtagtacgggctgaaatcatgcggtccttctctttaagcacaaacctccaggaatctctgcgctccaaagaagataatttgtatttacaaggccaccatcaccatcaccatcccatgccttggcgcgtgattaaaacccctttaactgaagccgtttccaccgctgactctcaaggtcgctttctgagcagcaccgaattgcaaattgctttcggtcgtctacgtcaagctaatgctggtttgcaagccgctaaagctctgaccgacaatgcccagagcttggtaaatggtgctgcccaagccgtttataacaaattcccctacaccacccaaacccaaggcaacaactttgctgcggatcaacggggtaaagacaagtgtgcccgggacatcggctactacctccgcatcgttacctactgcttagttgctggtggtaccggtcctttggatgagtacttgatcgccggtattgatgaaatcaaccgcacctttgacctctcccccagctggtatgttgaagctctgaaatacatcaaagctaaccacggcttgagtggcgatgcccgtgacgaagctaattcctacctcgattacgccatcaatgctctgagctag

SEQ ID NO: 38 - protein, 172 aa, synthetic construct
MFDVFTRVVSQADARGEYLSGSQLDALSATVAEGNKRIDSVNRITGNASAIVSNAARALFAEQPQLIQPGGNAYTSRRMAACLRDMEIILRYVTYATFTGDASVLEDRCLNGLRETYVALGVPGASVAAGVQKMKEAALDIVNDPNGITRGDCSAIVAEIAGYFDRAAAAVA

SEQ ID NO: 39 - protein, 30 aa, synthetic construct
SGYFKNQLYSGSIPEKIQLKADISRKIFTF

SEQ ID NO: 40 - protein, 5 aa, synthetic construct
REIPE

SEQ ID NO: 41 - protein, 29 aa, synthetic construct
GYFKNQLYSGSIPEKIQLKADISRKIFTF

SEQ ID NO: 42 - protein, 166 aa, synthetic construct [sequence omitted]

SEQ ID NO: 43 - protein, 161 aa, synthetic construct
KTPLTEAVSTADSQGRFLSSTELQIAFGRLRQANAGLQAAKALTDNAQSLVNGAAQAVYNKFPYTTQTQGNNFAADQRGKDKCARDIGYYLRIVTYCLVAGGTGPLDEYLIAGIDEINRTFDLSPSWYVEALKYIKANHGLSGDARDEANSYLDYAINALS

SEQ ID NO: 44 - DNA, 2572 bp, synthetic construct
  source 1..2572: mol_type = other DNA
ccctcattcccgatgaagacaatccccaacagggactactactgcgggagaggggctatgcggaaattgtccccattgccggtcgctatcacatcgacgaagaggaagccctggtgctagtgacggagtatgagaccatgaccaccattgaacggttctggtttgccaacccggatatgcgccttagaaccagtactgtgcaaaggtttgggggattcaataccgccacctactgtacggaaatgcgggtcaaagaggaaaacaccgtgtctgcgtcccctgcccctgcctacgaacaattttgtggctggtagccccgtttggttccagtatggggagctcgatcgccctgccaagccatctgaggaattggctaaccctgaatggaagggcgataattgtaataaattgtaatatttgccaacgattaggacccttcccccttattatttaaactaactaattatcaagctcttctttaagatcacggaggtttacacatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaggttattttaaaaaccaactttactcaggttccatacccgagaaaatccagcttaaagctgacatatctaggaaaattttcacattctaacgggagataccagaacaatgtgtgacttgcctcagacgcattctttgggaagccgacgcacactgatgctgctcgcccaaatgcgccggatctccttattctcctgtctcaaggatcggcatgacttcggcttccctcaggaggagtttggaaatcagttccaaaaggccgaaaccattccggtcctccatgaaatgattcaacagatctttaacttattcagtaccaaagacagcagtgcggcctgggacgaaacattactcgataaattctacacggaattataccaacagttgaacgacttagaagcctgtgtaatccaaggtgttggtgtcactgagactccattaatgaaagaagactctattctggccgtccgcaagtatttccagcgaatcacactgtatttgaaagagaaaaagtattctccgtgtgcgtgggaggtagtacgggctgaaatcatgcggtccttctctttaagcacaaacctccaggaatctctgcgctccaaagaagataatttgtatttacaaggccaccatcaccatcaccatcccatgccttggcgcgtgattgctcttcccctattgaactacgcccccaaaagtcaaaatgtgagggtagaaggttatgaaattggctctgaagagaagcctgttgttttcaccacggaaaacatcctctccagcagcgatatggataacctaatcgaggcggcctatcgtcaaatctttttccatgcgtttaagtgggaccgagaaaaagtccttgagtcccaactgcgtaacggccaaattactgtacgagattttgtgcggggcttgttgctttccaacaccttccgcaatagcttctacgaaaagaatagtaactaccgcttcgttgagcactgtgtacagaagattttagggcgggacgtttacagcgaacgggaaaaaattgcttggtccattgtggtcgcgaccaagggctatcaaggattaattgacgatttgctcaacagcgacgagtacctcaataactttggctatgacacggtgccctaccaacgtcgtcgcaaccttcccggtcgggaagcgggtgaattgccctttaacatcaaatctccccggtacgatgcctaccaccgtcgccaattgggcttcccccaaatcgtttggcagaacgaagtgcgtcgctttattccccaggagaaaaagctcaccgctggcaatccgatgaacttcttgggcatggcccgcagtatcaaccctgccgccaacaccattcccaaggtttccgcccaaaatatcaacatcgaagcttctgtgccccgtcgctag

SEQ ID NO: 45 - protein, 39 aa, synthetic construct
PSFPMKTIPNRDYYCGRGAMRKLSPLPVAITSTKRKPWC

SEQ ID NO: 46 - protein, 5 aa, synthetic construct
RSMRP

SEQ ID NO: 47 - protein, 90 aa, synthetic construct
PPLNGSGLPTRICALEPVLCKGLGDSIPPPTVRKCGSKRKTPCLRPLPLPTNNFVAGSPVWFQYGELDRPAKPSEELANPEWKGDNCNKL

SEQ ID NO: 48 - protein, 14 aa, synthetic construct
YLPTIRTLPPYYLN

SEQ ID NO: 49 - protein, 13 aa, synthetic construct
LIIKLFFKITEVY

SEQ ID NO: 50 - protein, 248 aa, synthetic construct
ALPLLNYAPKSQNVRVEGYEIGSEEKPVVFTTENILSSSDMDNLIEAAYRQIFFHAFKWDREKVLESQLRNGQITVRDFVRGLLLSNTFRNSFYEKNSNYRFVEHCVQKILGRDVYSEREKIAWSIVVATKGYQGLIDDLLNSDEYLNNFGYDTVPYQRRRNLPGREAGELPFNIKSPRYDAYHRRQLGFPQIVWQNEVRRFIPQEKKLTAGNPMNFLGMARSINPAANTIPKVSAQNINIEASVPRR

(19) United States
(12) Patent Application Publication
(10) Pub. No.: US 2024/0376174 A1
(43) Pub. Date: Nov. 14, 2024

(54) Heterohexameric Fusion Constructs for Protein Expression in Cyanobacteria

(71) Applicant: The Regents of the University of California, Oakland, CA (US)

(72) Inventors: Anastasios Melis, El Cerrito, CA (US); Diego Hidalgo Martinez, El Cerrito, CA (US); Nico Betterle, Pleasanton, CA (US)

(21) Appl. No.: 18/688,962

(22) PCT Filed: Sep. 2, 2022
(86) PCT No.: PCT/US2022/075894

§ 371 Date: Mar. 4, 2024

Related U.S. Application Data
(60) Provisional application No. 63/240,615, filed on Sep. 3, 2021.

(51) Int. Cl. C07K 14/555; C07K 14/33; C12N 1/20; C12N 15/74; C12R 1/00

(52) U.S. Cl. CPC C07K 14/555; C07K 14/33; C12N 1/205; C12N 15/74; C12R 2001/00

(57) ABSTRACT

This invention provides compositions and methods for providing high level expression of proteins of interest in cyanobacteria.

### CROSS-REFERENCE TO RELATED APPLICATIONS

This application claims priority benefit of U.S. Provisional Application No. 63/240,615, filed Sep. 3, 2021, which is incorporated by reference for all purposes.

### BACKGROUND OF THE INVENTION

In cyanobacteria, e.g., Synechocystis sp. PCC 6803 (Synechocystis), the phycobilisome (PBS) comprises the major light harvesting antenna complex of photosynthesis (Grossman et al. 1995). The PBS is composed of different proteins, which are grouped as allophycocyanin (AP) α and β subunits, phycocyanin (PC) α and β subunits, and several polypeptide linker proteins (Yamanaka et al. 1978; 1982; Ughy and Ajlani 2004; Watanabe and Ikeuchi 2013). These are organized as core AP cylinders and peripheral PC rods (Kirst et al. 2014). Covalently bound to the AP and PC proteins are bilins, open tetrapyrrole pigments that function in sunlight absorption and excitation energy transfer to the photosystem II (PSII) reaction center (Sidler 1994; Liu et al 2005). Sunlight absorbed by the PBS pigments is unidirectionally channeled from the peripheral rods, composed of PC subunit discs, to the core cylinders formed by AP subunits. The latter are found on the surface of the cyanobacterial thylakoid membranes, in close association with the membrane-bound photosystem II complexes. Peripheral phycocyanin rods extend from the core cylinders into the soluble cytoplasmic phase of the cyanobacteria (Kirst et al. 2014). Substantial amino acid resources are invested by the cell to construct the sizable PBS, comprising by far the most abundant proteins in the cell. Under nitrogen or sulfur nutrient deprivation, cyanobacteria undergo “chlorosis”, comprising a well-regulated developmental program of PBS degradation to serve as a source of needed nitrogen or sulfur nutrients for survival (Richaud et al 2001; Elmorjani and Herdman 1987; Collier and Grossman 1994).

Synechocystis possesses hemidiscoidal phycobilisomes, whereby PC is the only biliprotein that makes up the peripheral rods. The α (CpcA) and β (CpcB) subunits of PC dimerize into heterodimers, then they assemble into heterohexameric (α,β)₃ disks that are subsequently stacked to form the peripheral rod. The PC discs that are proximal to the AP core cylinders structurally and electronically couple to the core AP through the colorless CpcG1/G2 polypeptide linkers (Marsac and Cohen 1977; Kondo et al 2005; Bolte et al 2008; Ughy and Ajlani 2004). Additional colorless linker polypeptides ensure the structural stability of the middle PC disc (cpcC1 gene product), and that of the distal PC disc (cpcC2 gene product) in the PC rods (Yamanaka et al. 1978; 1982; Ughy and Ajlani 2004). Since PC is the major soluble protein in cyanobacteria, the operon where it is encoded (the cpc operon) has been an important target for protein expression studies by several investigators (Formighieri and Melis 2014; Zhou et al. 2014; Davies et al. 2014; Englund et al. 2016;

Betterle et al. 2020). The cpc operon encodes the CpcA, CpcB, CpcC1, CpcC2 and CpcD proteins. As mentioned, only the CpcA and CpcB subunits of PC bind the light absorbing bilin pigments, whereas the last 3 proteins serve as PC linker polypeptides.

In synthetic biology, including the generation of bioproducts in cyanobacteria, yield of process often depends on the concentration of the pathway-catalyzing recombinant enzymes. However, heterologous proteins are often difficult to express in the host cell, as they either are contained in inclusion bodies or are degraded by the cell. This has been a barrier to the meaningful application of plants and algae in synthetic biology, as it has resulted in low steady-state level of recombinant proteins (Demain and Vaishna 2009;Surzycki et al. 2009; Tran et al. 2009; Coragliotti et al. 2011; Gregory et al. 2013; Jones and Mayfield 2013; Rasala and Mayfield 2015; Baier et al. 2018), often much less than 0.1% of the plant tissue or alga protein content (Dyo and Purton 2018). This pitfall limits carbon partitioning toward the target pathway and negatively impacting rates and yield of product biosynthesis. Thus, the true over-expression of recombinant proteins functioning in heterologous biosynthetic pathways has been a barrier and a problem.

A cpcB*fusion construct between the highly-expressed cpcB gene in cyanobacteria and transgenes from plants, bacteria and human provided expression of stable, soluble, and active recombinant enzyme in Synechocystis at a level of up to 20% of the cellular protein content, irrespective of the plant (Formighieri and Melis 2015; 2017; Chaves et al. 2017), human (Betterle et al. 2020), or bacterial origin (Chaves et al. 2016; 2018; Zhang et al. 2021) of the heterologous protein. This was demonstrated with individual enzymes, including the isoprene (hemiterpene) synthase, a number of monoterpene and diterpene synthases from plants, human interferon and other cytokines, as well as the bacterial isopentenyl diphosphate isomerase and tetanus toxin fragment C, all of which were expressed to levels greater than 10% of the total cell protein. See, e.g., WO20201050968, WO2017205788, and WO2016210154.

### BRIEF SUMMARY OF SOME ASPECTS OF THE INVENTION

The present disclosure is based, at least in part, on the identification of the supramolecular structure, multimeric organization, and function of CpcB, CpcA, CpcG, and CpcC1 fusion proteins in Synechocystis that provide marked heterologous protein over-expression and function in these photosynthetic microorganisms.

In one aspect, the disclosure provides a method of producing a protein of interest in a cyanobacteria host cell, wherein the protein of interest is encoded by a recombinant expression unit comprising:
(i) a nucleic acid sequence encoding a fusion protein comprising the protein of interest fused at the carboxyl terminus or amino terminus of a cyanobacterial CpcB protein, wherein the fusion protein is expressed as a component of functional (α,β*P)₃CpcG or (α,P*β)₃CpcG heterohexameric discs, where α is a cyanobacterial CpcA phycocyanin subunit protein, β is a cyanobacterial CpcB phycocyanin subunit protein, asterisk denotes fusion, P is the protein of interest, and CpcG is a phycocyanin linker polypeptide;
(ii) a nucleic acid sequence encoding the CpcA phycocyanin subunit protein; and
(iii) a nucleic acid sequence encoding the CpcG phycocyanin linker polypeptide; and wherein the method comprises:
(a) culturing the cyanobacterial host cell comprising the recombinant expression unit under conditions in which functional heterohexameric discs comprising the fusion protein are expressed; and
(b) purifying the hexameric discs comprising the protein of interest to at least 90% (w/w) purity. In some embodiments, the recombinant expression unit is operably linked to an endogenous cyanobacteria cpc promoter. In some embodiments, the fusion protein comprises a protease cleavage site between the CpcB protein and the polypeptide of interest.

In an additional aspect, the disclosure provides a recombinant cyanobacterial host cell comprising a (α,β)₃CpcG heterohexameric disc, which not to be bound by theory, serves as a carrier of expressed recombinant (or native) proteins of interest, wherein α is a cyanobacterial CpcA phycocyanin subunit protein, β is a cyanobacterial CpcB phycocyanin subunit protein, and CpcG is a phycocyanin linker polypeptide; and wherein at least one cyanobacterial protein selected from CpcB, CpcA, and CpcG is fused to a first protein of interest to be expressed in the cyanobacterial host cell and a second cyanobacterial protein, different from the first, selected from CpcB, CpcA, and CpcG is fused to a second protein of interest to be expressed in the cyanobacterial host cell, wherein the second protein of interest may be the same protein as the first protein of interest or a different protein. In some embodiments, the first protein of interest is fused to a CpcB and the second protein of interest is fused to CpcA. In some embodiments, the first protein of interest is fused to a CpcB and the second protein of interest is fused to CpcG. In some embodiments, the first protein of interest is fused to a CpcA and the second protein of interest is fused to CpcG.

In a further aspect, the disclosure features a recombinant cyanobacterial host cell comprising a (α,β)₃CpcG heterohexameric disc, wherein α is a cyanobacterial CpcA phycocyanin subunit protein, β is a cyanobacterial CpcB phycocyanin subunit protein, and CpcG is a phycocyanin linker polypeptide, and wherein a protein of interest is fused to the N-terminus or C-terminus of CpcG; or the protein of interest if fused to the N-terminus of CpcB or the N-terminus of CpcA.

In another aspect, the disclosure features a recombinant cyanobacterial host cell comprising a first and a second fusion protein, wherein the first and the second fusion protein are encoded by a recombinant expression unit that expresses one or more proteins of interest, wherein the first fusion protein comprises a first protein of interest fused at the carboxyl terminus or amino terminus of a CpcB protein and the second fusion protein comprises a second protein of interest, which may be the same or different from the first protein of interest, fused at the carboxyl terminus or amino terminus of a CpcA protein; and the first and second fusion proteins are expressed as a component of functional (α*P2,β*P1)₃CpcG or (α*P2,P1*β)₃CpcG or (P2*α,β*P1)₃CpcG or (P2*α,P1*β)₃CpcG heterohexameric discs, wherein: α is a cyanobacterial CpcA phycocyanin subunit protein, β is a cyanobacterial CpcB phycocyanin subunit protein, asterisk denotes fusion, P1 is the first protein of interest, P2 is the second protein of interest, and CpcG is a phycocyanin linker polypeptide. In some embodiments, the first fusion protein comprises a protease cleavage site between CpcB and the first polypeptide of interest and the second fusion protein comprises a cleavage site between CpcA and the second polypeptide of interest. In some embodiments, the recombinant expression unit is operably linked to an endogenous cyanobacteria cpc promoter.

In an additional aspect, the disclosure features a recombinant cyanobacteria host cell comprising a first and a second fusion protein, wherein the first and the second fusion protein are encoded by a recombinant expression unit that expresses one or more proteins of interest, wherein the first fusion protein comprises a first protein of interest fused at the carboxyl terminus or amino terminus of a CpcB protein and the second fusion protein comprises a second protein of interest, which may be the same or different from the first protein of interest, fused at the carboxyl terminus or amino terminus of a CpcA protein, and the first and second fusion proteins are expressed as a component of functional (α*P2,β*P1)₃CpcC1 or (α*P2,P1*β)₃CpcC1 or (P2*α,β*P1)₃CpcC1 or (P2*α,P1*β)₃CpcC1 heterohexameric discs. wherein: α is a cyanobacterial CpcA phycocyanin subunit protein, β is a cyanobacterial CpcB phycocyanin subunit protein, asterisk denotes fusion, P1 is the first protein of interest, P2 is the second protein of interest, and CpcC1 is a phycocyanin linker polypeptide. In some embodiments, the first fusion protein comprises a protease cleavage site between CpcB and the first polypeptide of interest and the second fusion protein comprises a cleavage site between CpcA and the second polypeptide of interest. In some embodiments, the recombinant expression unit is operably linked to an endogenous cyanobacteria cpc promoter.

In a further aspect, the disclosure features a recombinant cyanobacterial host cell comprising a first and a second fusion protein, wherein the first and the second fusion protein are encoded by a recombinant expression unit that expresses one or more proteins of interest, wherein the first fusion protein comprises a first protein of interest fused at the carboxyl terminus or amino terminus of a CpcG protein and the second fusion protein comprises a second protein of interest, which may be the same or different from the first protein of interest, fused at the carboxyl terminus or amino terminus of a CpcB protein: and the first and second fusion proteins are expressed as a component of functional (α,β*P2)₃CpcG*P1 or (α,P2*β)₃CpcG*P1 or (α,β*P2)₃P1*CpcG or (α,P2*β)₃P1*CpcG heterohexameric discs. wherein: α is a cyanobacterial CpcA phycocyanin subunit protein, β is a cyanobacterial CpcB phycocyanin subunit protein, asterisk denotes fusion, P1 is the first protein of interest, P2 is the second protein of interest, and CpcG is a phycocyanin linker polypeptide. In some embodiments, the first fusion protein comprises a protease cleavage site between CpcG and the first polypeptide of interest and the second fusion protein comprises a cleavage site between CpcB and the second polypeptide of interest. In some embodiments, the recombinant expression unit is operably linked to an endogenous cyanobacteria cpc promoter.

The disclosure additionally features a recombinant cyanobacterial host cell comprising a first and a second fusion protein, wherein the first and the second fusion protein are encoded by a recombinant expression unit that expresses one or more proteins of interest, wherein the first fusion protein comprises a first protein of interest fused at the carboxyl terminus or amino terminus of a CpcG protein and the second fusion protein comprises a second protein of interest, which may be the same or different from the first protein of interest, fused at the carboxyl terminus or amino terminus of a CpcA protein; and the first and second fusion proteins are expressed as a component of functional (α*P2,β)₃CpcG*P1 or (P2*α,β)₃CpcG*P1 or (α*P2,β)₃P1*CpcG or (α*P2,β)₃P1*CpcG heterohexameric discs, wherein: a is a cyanobacterial CpcA phycocyanin subunit protein, β is a cyanobacterial CpcB phycocyanin subunit protein, asterisk denotes fusion, P1 is the first protein of interest, P2 is the second protein of interest, and CpcG is a phycocyanin linker polypeptide. In some embodiments, the first fusion protein comprises a protease cleavage site between CpcG and the first polypeptide of interest and the second fusion protein comprises a cleavage site between CpcA and the second polypeptide of interest. In some embodiments, the recombinant expression unit is operably linked to an endogenous cyanobacteria cpc promoter.

In some embodiments, the cyanobacteria are single celled cyanobacteria, such as a Synechococcus sp., a Thermosynechococcus sp., a Synechocystis sp., or a Cyanothece sp. In some embodiments, the cyanobacteria are micro-colonial cyanobacteria, such as a Gloeocapsa magma, Gloeocapsa phylum, Gloeocapsa alpicola, Gloeoepasa atrata, Chroococcus spp., or Aphanothece sp. In some embodiments, the cyanobacteria are filamentous cyanobacteria, such as an Oscillatoria spp., a Nostoc sp., an Anabaena sp., or an Arthrospira sp. In some embodiments, at least one of the proteins of interest is isoprene synthase, a β-phellandrene synthase, a geranyl diphosphate synthase, a geranyl linalool synthase, human interferon α-2 or other cytokine, a cholera toxin B (CtxB) protein, or a tetanus toxin fragment C (TTFC). In some embodiments, a protein of interest is G-CSF, GM-CSF, MCP1 sCD40L, TGF-alpha, EGF, FGF-2, Flt-3L, INF-apha2, INF-gamma, IL-10, IL-15, IL-17, IL-1beta, IL-2, IL-6, IL-8, IL-10, IL-15, IL-17, IL-1beta, IL-2, IL-6, IL-8, IP-10, MIP-1beta, PDGF-AA, TNF-alpha, or VEGF. In a further aspect, the disclosure provides a cyanobacteria culture comprising a host cell as described herein, e.g., in this paragraph.

In a further aspect, the disclosure features a method of producing a first and a second protein of interest, the method comprising growing a cyanobacteria culture as described herein under conditions in which the first and the second protein of interest are expressed.

In an additional aspect, the disclosure provides a recombinant cyanobacteria host cell comprising a recombinant expression unit comprising:
(i) a nucleic acid sequence encoding a cyanobacterial CpcA phycocyanin subunit protein,
(ii) a nucleic acid sequence encoding a cyanobacterial CpcB phycocyanin subunit protein, and
(iii) a nucleic acid sequence encoding a fusion protein comprising a protein of interest fused at the carboxyl terminus or amino terminus of a cyanobacterial CpcG polypeptide,
wherein the fusion protein is expressed as a component of functional heterohexameric discs comprising the cyanobacterial CpcA phycocyanin subunit protein, the cyanobacterial CpcB phycocyanin subunit protein, and the fusion protein fused at the carboxyl terminus or amino terminus of the cyanobacterial CpcG polypeptide. In some embodiments, the recombinant expression unit is operably linked to an endogenous cyanobacteria cpc promoter. In some embodiments, the fusion protein comprises a protease cleavage site between CpcG and the protein of interest. In some embodiments, the cyanobacteria are single celled cyanobacteria, such as a Synechococcus sp., a Thermosynechococcus sp., a Synechocystis sp., or a Cyanothece sp. In some embodiments, the cyanobacteria are micro-colonial cyanobacteria, such as a Gloeocapsa magma, Gloeocapsa phylum, Gloeocapsa alpicola, Gloeocpasa atrata, Chroococcus spp., or Aphanothece sp. In some embodiments, the cyanobacteria are filamentous cyanobacteria, such as an Oscillatoria spp., a Nostoc sp., an Anabaena sp., or an Arthrospira sp. In some embodiments, at least one of the proteins of interest is isoprene synthase, a β-phellandrene synthase, a geranyl diphosphate synthase, a geranyl linalool synthase, human interferon α-2 protein or other cytokine, a CtxB protein, or TTFC. In some embodiments, a protein of interest is G-CSF, GM-CSF, MCP1 sCD40L, TGF-alpha, EGF, FGF-2, Flt-3L, INF-apha2, INF-gamma, IL-10, IL-15, IL-17, IL-1beta. IL-2, IL-6, IL-8, IL-10, IL-15, IL-17, IL-1beta, IL-2, IL-6, IL-8, IP-10, MIP-1beta, PDGF-AA, TNF-alpha, or VEGF. In a further aspect, the disclosure further provides a cyanobacteria culture comprising a host cell as described herein, e.g., in this paragraph.

In one aspect, the disclosure also features a heterohexameric disc preparation at least 90% pure, comprising heterohexameric discs comprising a cyanobacterial CpcA phycocyanin subunit protein, a cyanobacterial CpcB phycocyanin subunit protein, and CpcG or CpcC1 phycocyanin linker polypeptides, wherein at least one of CpcA, CpcB, CpcG, or CpcC1 is fused at a C-terminal or N-terminal end to a protein of interest expressed in cyanobacteria. In some embodiments, the protein of interest is linked to the C-terminal end of CpcB.

### BRIEF DESCRIPTION OF THE DRAWINGS

FIG. 1A-E: Schematic overview of DNA maps of the cpc operon in wild type and Synechocystis transformants. (A) The native cpc operon, as it occurs in wild type Synechocystis. This DNA operon configuration and sequence are referred to as the wild type (WT). (B) Replacement of the cpcB gene, which encodes the β-subunit of phycocyanin, with fusion construct cpcB*6xHis*tev*IFN harboring the interferon α-2 (IFN)-encoding DNA. This cpcB*IFN fusion construct was followed by the chloramphenicol (cmR) resistance cassette in an operon configuration. (C) Replacement of the cpcB gene with fusion construct cpcB*6xHis*tev*TTFC harboring the tetanus toxin fragment C (TTFC)-encoding DNA. This cpcB*TTFC fusion construct was followed by the spectinomycin (smR) resistance cassette in an operon configuration. (D) Replacement of the cpcB gene with minimal fusion construct cpcB*6xHis*tev harboring no transgene for a heterologous protein. This minimal cpcB* fusion construct was followed by the spectinomycin (smR) resistance cassette in an operon configuration. This minimal cpcB* fusion construct was generated upon deletion of the TTFC gene from the cpcB*6xHis*tev*TTFC construct (C). (E) Replacement of the entire cpc operon native genes (cpcB, cpcA, cpcC2, cpcC1 and cpcD) with the kanamycin resistance resistant cassette (nptI). This construct is referred to as Δcpc.

FIG. 2: Genomic DNA PCR analysis testing for transgenic DNA copy homoplasmy in Synechocystis transformants. DNA from the WT and transformants CpcB*, CpcB*IFN and CpcB*TTFC was amplified using cpcB_fw and cpcA_rv primers (FIG. 1). The WT yielded a 298 bp PCR product, whereas products of 1325, 1488, 2678 bp were observed and for the cpcB*, cpcB*IFN, cpcB*TTFC strains, respectively. Three independent cpcB* transformant lines were tested, as this transgenic strain was not used before. Transformants cpcB*IFN and cpcB*TTFC have been tested for homoplasmy in recent work from this lab (Betterle et al. 2020; Zhang et al. 2021). Absence of 298 bp products in the transformants is evidence of DNA copy homoplasmy in these strains. Primer sequences are provided in Example 1.

FIG. 3: SDS-PAGE Zinc and Coomassie staining of total cell extracts. The left panel shows the fluorescence of the tetrapyrrole bilin pigments that are covalently bound to the allophycocyanin, phycocyanin subunits CpcA and CpcB, and the CpcB*, CpcB*IFN, and CpcB*TTFC constructs (marked by asterisk). Note that some CpcA Zn-fluorescence remains in the 17 kD position, whereas all CpcB Zn-fluorescence is lost in the CpcB*, CpcB*IFN and CpcB*TTFC transformants. The latter have been replaced by fluorescing bands at 21, 36 and 73 kD, respectively). The right panel shows the same SDS-PAGE profile of total protein extracts, now stained with Coomassie. The asterisks indicate the bands that match the analysis of the Zn fluorescence.

FIG. 4: Zn-chromophore fluorescence profile of total cell extracts. SDS-PAGE loaded with a variable range of Chl amounts of WT (lane 1-4), CpcB*IFN (lane 5-8) and CpcB*TTFC (lane 9-12). Loadings were as follows: WT (lane 1-4): 0.025, 0.05, 0.075, and 0.1 μg Chl. CpcB*IFN (lane 5-8): 1.5, 1.75, 2, and 2.25 μg Chl. CpcB*TTFC (lane 9-12): 1.25, 1.5, 1.75, and 2 μg Chl. The horizontal lines indicate the Zn-chromophore fluorescence bands used for the calculation of the fluorescence intensity signal and, subsequently, the CpcB/CpcA fluorescence yield ratio determination.

FIG. 5: A side-by-side SDS-PAGE analysis of the protein profile from the total Synechocystis cell extracts and those proteins eluted from the column affinity chromatography. Left side shows the profile of total protein of WT, CpcB*IFN and CpcB*TTFC cell extract. Marked with asterisk are the CpcB and CpcA proteins for the WT, the 36 kD CpcB*IFN fusion, and the 73 kD CpcB*TTFC fusion. The right side, shows the eluted fractions from the affinity column purification experiments. No wild type proteins were retained/eluted from the column. The CpcB*IFN and CpcB*TTFC transformants showed the anticipated 36 and 73 kD bands. Additionally, a ˜27 kD polypeptide and the CpcA protein were selectively and reproducibly eluted from this column chromatography.

FIG. 6: Quantification of proteins selectively eluted from the affinity column purification experiments of the CpcB*IFN strain. SDS-PAGE lanes were loaded with increasing volumes of the eluted fraction, shown on top of the gel for each of the lanes. The protein profile was visualized upon staining with Coomassie and band intensity was quantified upon scanning of the stained bands. Corrected for the different molecular sizes of the proteins, this analysis revealed a constant CpcB*IFN/CpcG/CpcA=3:1:3 ratio.

FIG. 7: Quantification of proteins selectively eluted from the affinity column purification experiments of the CpcB*TTFC strain. SDS-PAGE lanes were loaded with increasing volumes of the eluted fraction, shown on top of the gel for each of the lanes. The protein profile was visualized upon staining with Coomassie and band intensity was quantified upon scanning of the stained bands. Corrected for the different molecular sizes of the proteins, this analysis revealed a constant CpcB*TTFC/27kD/CpcA=3:1:3 ratio.

FIG. 8: Left panel, shows the Zn-chromophore fluorescence profile of eluted protein fractions from the CpcB* (21 kD), CpcB*IFN (36 kD), cpcB*TTFC (73 kD). No proteins were eluted from the affinity column purification of the WT. As a control, a total wild type protein extract (WT total-no affinity column purification) was also loaded, showing the Zn-chromophore fluorescence of the CpcB (19 kD) and CpcA (17 kD) proteins. The latter was present just in the total protein extract from WT strain, which was used as a reference due to the fact that WT extracts did not show any protein after affinity chromatography. A densitometric analysis of the Zn-fluorescence bands was carried out to measure the CpcB/CpcA ratio, as evidenced from the fluorescence intensity of the respective bands. These ratios were found to be: CpcB*/CpcA=1.21±0.12. CpcB*IFN/CpcA=1.30±0.04 and CpcB*TTFC/CpcA=1 40±0.13. The right panel shows the SDS-PAGE Coomassie stain of the same gel, revealing the same protein profile, as the one shown by the Zn-chromophore fluorescence. In addition, the Coomassie stain also showed presence of the CpcG protein, which lacked a Zn-chromophore fluorescence emission.

FIG. 9: Native PAGE analysis of eluted proteins from the WT and the strains harboring the various CpcB*Fusion constructs. Left panel shows the Zn-chromophore fluorescence profile of eluted fractions. Note that only a single Zn-fluorescence band is observed. Right panel shows a Coomassie stain of the respective Native-PAGE analysis proteins, where again a single Coomassie stained band is observed. The different electrophoretic mobilities correspond to the calculated (α,β*P)₃CpcG heterohexamer molecular weights of CpcB*=140 kD, CpcB*IFN=188 kD, and CpcB*TTFC=296 kD. These results suggested that the eluted fractions comprise a complex of the associated Fusion, CpcG, and CpcA proteins. Ten μL of elution fraction was used to load the PAGE lanes.

FIG. 10: Schematic presentation of the minimal stable (α,β*IFN)₃CpcG and (α,β*TTFC)₃CpcG heteroexameric complex configuration characterized as described in the EXAMPLES section. The experimental work detailed herein showed that CpcB*IFN and CpcB*TTFC assemble with other native subunits into a higher order complex rather than occurring as freely-soluble fusion proteins in the cytosol of cyanobacteria. The higher order complex comprises the CpcA α-subunits and CpcB β-subunits, which are represented by circles and shown to form a tight heterohexamer disc (David et al. 2011) labeled with α and β, respectively. IFN and TTFC are employed in this schematic as examples of recombinant proteins; however, any protein of interest to be expressed in cyanobacteria may be expressed as described herein. Further, although the recombinant proteins are shown fused to the CpcB β-subunits in the schematic depicted in this figure, the proteins can alternatively be fused to CpcA α-subunits or to the CpcG protein. The CpcG protein (G) is proposed to occupy the disc center of the (α,β*IFN)₃ and (α,β*TTFC)₃ complexes. Note that the heterologous fusion proteins localize to the periphery and emanate radially from the (α,β*IFN)₃ or (α,β*TTFC)₃ disc. A structure as depicted here thus can be employed as a platform cyanobacterial carrier of expressed recombinant and native proteins of interest.

FIG. 11: Absorbance spectra of wild type and CpcB*Fusion construct cell suspensions. This figure shows the pigment profile in WT, CpcB*, CpcB*IFN, CpcB*TTFC and Δcpc strains. The absorbance spectra were normalized to the Chl max at 678 nm so that differences of the absorbance at 620 nm could be more readily seen among the various transformants.

FIG. 12. The phycocyanin configuration in the modified phycobilisome, which is encountered in strains harboring the CpcB*Fusion protein complexes. The (α,β*TTFC)₃CpcG heterohexameric complexes are structurally and functionally coupled to the allophycocyanin core cylinders of the modified phycobilisome so as to enable excitation energy transfer from PC to AP and then to the PSII reaction center. TTFC is depicted as the recombinant fused protein to the CpcB β-subunits in this schematic, although it could be IFN, or any of the other heterologous fusion protein discussed in the literature. Note that the heterologous fusion protein is exposed to the aqueous medium in the Synechocystis cytosol.

FIG. 13A-C. Schematic overview of DNA maps of the cpc operon in wild type and Synechocystis transformants. (A) The native cpc operon, as it occurs in wild type Synechocystis. This DNA operon configuration and sequence is referred to as the wild type (WT). (B) Replacement of the cpcA gene, which encodes the CpcA α-subunit of phycocyanin, with fusion construct cpcA*S7*6xHis*tev*IFN harboring the interferon α-2(IFN)-encoding DNA. This construct is referred to as cpcA*IFN. The cpcA*IFN fusion construct was followed by the chloramphenicol (cmR) resistance cassette in an operon configuration. (C) Replacement of the entire cpc operon native genes (cpcB, cpcA, cpcC2, cpcC1 and cpcD) with the kanamycin resistance cassette (nptI). This construct is referred to s Δcpc.

FIG. 14. SDS-PAGE Coomassie staining and Zinc-chromophore fluorescence of total cell extracts from wild type (WT) and CpcA*IFN transformants. The left panel shows the SDS-PAGE profile of total protein extracts stained with Coomassie. Marked by arrows are the phycocyanin subunits CpcA and CpcB, the chloramphenicol resistance CmR, and the large subunit of Rubisco (RbcL). Also marked is the CpcA*IFN fusion protein (34 kDa; marked by asterisks). The right panel shows the fluorescence of the tetrapyrrole bilin pigments that are covalently bound to the allophycocyanin APC, and phycocyanin CpcA and CpcB subunits. The CpcA*IFN fluorescence is below the detection limit of this analytical system, probably as a result of the low-yield emission by the Zn-chromophore in the CpcA*IFN protein. Note that CpcB Zn-chromophore fluorescence is present in the 19 kDa position, whereas all CpcA fluorescence is missing from the 17 kDa position in the CpcA*IFN transformants.

FIG. 15. SDS-PAGE Coomassie stain and Western blot analysis of total cell extracts from wild type (WT) and CpcA*IFN transformants. The left panel shows the SDS-PAGE profile of total protein extracts stained with Coomassie. Marked by arrows are the phycocyanin subunits CpcA and CpcB, the CmR, and the large subunit of Rubisco (RbcL). Also marked is the CpcA*IFN fusion protein (34 kDa, marked by asterisk). The right panel shows a Western blot analysis of total cell protein extracts probed with anti-IFN specific antibodies. Substantial amounts of distinct higher molecular weight cross-reactions with the anti-IFN antibodies were detected and attributed to aggregates of the (α*IFN)₃ CpcG1 complex that did not dissociate by the detergent action. Notice the absence of IFN from the wild type (WT) and the cross-reaction of the antibodies with a band migrating to about 34 KD, attributed to the CpcA*IFN fusion protein.

FIG. 16. Absorbance spectra of wild type. CpcA*IFN fusion construct, and phycocyanin-less Δcpc mutant, measured with Synechocystis lysed cell suspensions. The absorbance spectra were normalized to the chlorophyll max at 678 nm so that differences of the absorbance at 620 nm could be more readily seen among the various strains. The phycocyanin absorbance of the mutants relative to that measured in the wild type was CpcA*IFN=8.25% and Δcpc=0%.

FIG. 17A-D. Schematic overview of DNA maps of the cpc operon and the CpcG1 locus in wild type and Synechocystis transformants. (A) The native cpc operon, as it occurs in wild type Synechocystis. This DNA operon configuration and sequence is referred to as the wild type (WT). (B) Replacement of the cpcG1 gene, which encodes the colorless CpcG128.9 kDa linker polypeptide, with fusion construct cpcG1*S7*6xHis*tev*IFN (referred to as cpcG1*IFN). The CpcG1 polypeptide serves to anchor the proximal phycocyanin (α,β)₃(α,β)₃ disk to the phycobilisome core cylinders. The cpcG1*IFN fusion DNA was followed by the chloramphenicol (cmR) resistance cassette in an operon configuration. (C) Replacement of the cpcB gene with fusion construct cpcB*S7*6xHis*tev*TTFC (referred to as cpcB*TTFC) harboring the tetanus toxin fragment C (TTFC)-encoding DNA. This cpcB*TTFC fusion construct was followed by the spectinomycin (smR) resistance cassette in an operon configuration. In a double transformant configuration, the cpcB*TTFC strains also carried the cpcG1*S7*6xHis*tev*IFN fusion construct (double fusion transformants) (D) Replacement of the entire cpc operon native genes (cpcB, cpcA, cpcC2, cpcC1 and cpcD)) with the kanamycin resistance cassette (nptI). This construct is referred to as Δcpc.

FIG. 18. Genomic DNA PCR analysis testing for transgenic DNA copy homoplasmy in Synechocystis transformants. DNA from the WT, transformants cpcG1*IFN and double transformant cpcB*TTFC+cpcG1*IFN was amplified using cpcG1 5′_fw (5′-ATTGCCGGTCGCTATCACAT-3′) and cpcG1 3′_rv (5′-TGTCCAGAGGGAGACACCAA-3′) primers. The WT yielded 1,866 bp PCR products, whereas products of 3,197 bp were generated from the cpcG1*IFN and cpcB*TTFC+cpcG1*IFN strains. Three independent cpcG1*IFN and cpcB*TTFC+cpcG1*IFN transformant lines were tested, as these transgenic strains were not used earlier in this disclosure. Absence of 1,866 bp products in the transformants is evidence of DNA copy homoplasmy in these strains.

FIG. 19. SDS-PAGE Coomassie stain and Zinc-chromophore fluorescence of total cell extracts from wild type (WT), cpcG1*IFN and cpcB*TTFC+cpcG1*IFN strains. The left panel shows the SDS-PAGE profile of total protein extracts stained with Coomassie. Marked by arrows are the phycocyanin CpcA, CpcB, and allophycocyanin APC subunits, the CmR, and the large subunit of Rubisco (RbcL). Also marked are the abundantly-expressed CpcG1*IFN (˜46 kDa) and CpcB*TTFC (˜73 kDa) fusion protein (marked by arrows), and the CpcC1/CpcC2 middle/distal phycocyanin disc linker (marked by asterisks). Note the lower abundance of these linkers in the CpcB*TTFC+CpcG1*IFN double fusion strains. The right panel shows the Zn-chromophore fluorescence of the tetrapyrrole bilin pigments that are covalently bound to the allophycocyanin (APC), and phycocyanin subunits CpcA and CpcB. The ˜46 kDa CpcG1*IFN fusion protein has no bilin pigments, hence no fluorescence. The CpcB*TTFC (˜73 kDa) fusion protein shows a distinct CpcB Zn-chromophore fluorescence in the ˜73 kDa electrophoretic migration position.

FIG. 20. Western blot analysis of total cell extracts from wild type (WT), cpcG1*IFN and cpcB*TTFC+cpcG1*IFN strains. The left panel shows total cell proteins probed with anti-IFN specific antibodies. Note the absence of IFN from the wild type (WT) and the specific cross-reaction of the antibodies with a band migrating to about 46 kD. attributed to the CpcG1*IFN fusion protein. The right panel shows total cell proteins probed with anti-TTFC specific antibodies. Note the absence of cross-reaction with proteins from the wild type (WT) and the CpcG1*IFN mutant, and the specific cross-reaction of the anti-TTFC antibodies with a band migrating to about 73 kDa, attributed to the CpcB*TTFC fusion protein.

FIG. 21. Native PAGE analysis of affinity chromatography-eluted proteins from wild type (WT), cpcG1*IFN and cpcB*TTFC+cpcG1*IFN containing strains. Left panel shows a Coomassie stain of the respective Native-PAGE analysis proteins, where protein bands are observed migrating to 312, 266, and 185 kDa electrophoretic mobility positions in the cpcB*TTFC+cpcG1*IFN containing strain only. The three different electrophoretic mobilities may reflect the presence of (α,β*TTFC)₃CpcG1*IFN (MW=316 kDa), (α,β*TTFC)₃(MW=270 kDa), i.e., loss of the CpcG1*IFN during the fractionation/isolation process, and (α,β*TTFC)₂ (MW=180 kDa), i.c., loss of both the CpcG1*IFN and a portion the (α,β*TTFC): heterohexameric disc during the fractionation/isolation process.

FIG. 22. SDS-PAGE Coomassie staining and Western blot analysis of affinity chromatography-eluted proteins from wild type (WT), cpcG1*IFN and cpcB*TTFC+ cpcG1*IFN containing strains. The left panel shows a Coomassie stain of the respective Native-PAGE analysis proteins, where protein bands are observed migrating to 312, 266, and 185 kDa positions in the cpcB*TTFC+cpcG1*IFN containing strain only. The middle panel shows a Western blot analysis of the affinity chromatography-eluted proteins with anti-IFN specific antibodies. There was a single cross-reaction of the anti-IFN antibodies with proteins at the 312 kDa position, attributed to the (α,β*TTFC)₃CpcG1*IFN protein complex. Protein bands migrating to 266 kDa and 180 kDa did not cross react with the anti-IFN antibodies. As such, they are postulated not contain IFN and likely comprise (α,β*TTFC)₃=270 kD and (α,β*TTFC)₂=180 kDa complexes. The right panel shows a Western blot analysis of the affinity chromatography-eluted proteins with anti-TTFC specific antibodies. In this case, there were cross-reactions of the anti-TTFC antibodies with all three protein complexes eluted from the differential affinity chromatography approach.

FIG. 23. Absorbance spectra of wild type (WT), cpcG1*IFN and cpcB*TTFC+cpcG1*IFN strains containing fusion constructs, and the phycocyanin-less Δcpc mutant, measured with Synechocystis lysed cell suspensions. The absorbance spectra were normalized to the chlorophyll max at 678 nm so that differences of the absorbance at 620 nm could be more readily discerned among the various transformants. The phycocyanin absorbance of the mutants relative to that measured in the wild type was cpcB*TTFC+cpcG1*IFN (TTFC & IFN)=11.5%, CpcG1*IFN (G1*IFN)=68.4%, and Δcpc=0%.

FIG. 24. (Upper) Folding model of the cpcB*S7*6xHis*tev*TTFC fusion protein. (Lower) Structural presentation of the CpcA, CpcB (α,β)₃ disc with the fused TTFC recombinant protein (α,β*TTFC)₃ radially emanating from the (α,β)₃ heterohexamer. Assembly and function of the native (α,β)₃ heterohexameric complex suggests that the corresponding heterologous fusion proteins localize away from the disc center and likely place to the periphery or emanate radially from the (α,β+TTFC)₃ discs, thus exposed to the medium.

FIG. 25. Folding model of the cpcG1*S7*6xHis*tev*IFN fusion protein.

FIG. 26. Model of the partially-assembled phycocyanin rod, comprising the proximal to allophycocyanin (α,β)₃(α,β)₃ dimer disc (denoted as P-(α,β)₆)), and the middle (α,β)₃(α,β)₃ dimer disc (denoted as M-(α,β)₆)). Also shown is placement of the wiry CpcG1 linker with the C-terminus portion of the protein exposed at the proximal end of the rod (Dominguez Martin et al. 2022). The latter helps bind the phycocyanin rods onto the allophycocyanin core cylinders, conferring a functional excitation energy transfer process from phycocyanin to allophycocyanin and thence to the chlorophyll antenna of PSII. The fusion of IFN at the C-terminus of the CpcG1 protein causes interference with the proper binding and function of the phycocyanin rod in these transformant strains. (A, B, C) Different possible orientations of the (α,β)₃ heterohexamer discs with respect to each other in the truncated P-(α,β)₆ and M-(α,β)₆ phycocyanin rod.

FIG. 27A-B. Schematic overview of DNA maps of the modified cpcA locus in the cpc operon and of the cpcG1 locus in Synechocystis transformants. (A) Replacement of the cpcA gene, which encodes the CpcA α-subunit of phycocyanin, with fusion construct cmR-IFN*tev*6xHis**S7*cpcA (referred to as IFN*cpcA). This IFN*cpcA fusion construct was preceded by the chloramphenicol (cmR) resistance cassette in an operon configuration. (B) Replacement of the Synechocystis native cpcG1 gene, which encodes the colorless CpcG1 28.9 kDa linker polypeptide, with fusion construct cmR-IFN*tev*6xHis*S7*cpcG1 (referred to as IFN*cpcG1). The CpcG1 polypeptide serves to anchor the proximal phycocyanin (α,β)₃(α,β)₃ dimer disk to the phycobilisome core cylinders. The IFN*cpcG1 fusion DNA was preceded by the chloramphenicol (cmR) resistance cassette in an operon configuration.

FIG. 28. SDS-PAGE Coomassie stain and Western blot analysis of total cell extracts from wild type (WT) and IFN*CpcA transformants. The left panel shows the SDS-PAGE profile of total protein extracts stained with Coomassie. Marked by arrows are the electrophoretic mobility positions of the phycocyanin subunits CpcA and CpcB, and the large subunit of Rubisco (RbcL). Also marked is the IFN*CpcA fusion protein (34 kDa, marked by asterisk). The right panel shows a Western blot analysis of total cell protein extracts from the same samples probed with anti-IFN specific antibodies. Notice the absence of IFN from the wild type (WT) and the cross-reaction of the antibodies with a band migrating to about 34kD, attributed to the IFN*CpcA fusion protein. The latter accounted for about 3-5% of the total cell protein in these measurements.

FIG. 29. SDS-PAGE Coomassie stain and Western blot analysis of total cell extracts from wild type (WT) and IFN*CpcG1 transformants. The left panel shows the SDS-PAGE profile of total protein extracts stained with Coomassie. Marked by arrows are the electrophoretic mobility positions of the phycocyanin subunits CpcA and CpcB, and the large subunit of Rubisco (RbcL). Also marked is the IFN*CpcG1 fusion protein (46 kDa, marked by asterisk). The right panel shows a Western blot analysis of total cell protein extracts from the same samples probed with anti-IFN specific antibodies. Notice the absence of IFN from the wild type (WT) and the cross-reaction of the antibodies with a band migrating to about 46kDa, attributed to the IFN*CpcG1 fusion protein. The latter accounted for about 3-5% of the total cell protein in these measurements.

### DETAILED DESCRIPTION OF THE INVENTION

The term “naturally-occurring” or “native” as used herein as applied to a nucleic acid, a protein, a cell, or an organism, refers to a nucleic acid, protein, cell, or organism that is found in nature. For example, a polypeptide or polynucleotide sequence that is present in an organism that can be isolated from a source in nature and which has not been intentionally modified by a human in the laboratory is naturally occurring.

A polynucleotide sequence is “heterologous to” a second polynucleotide sequence if it originates from a foreign species, or, if from the same species, is modified by human action from its original form. For example, a polynucleotide sequence is “heterologous” to a host cell if it is operably linked to a promoter that differs from its native promoter in the host cell, or if it is different in sequence from the the native sequence in the host cell.

The term “recombinant” polynucleotide or nucleic acid refers to one that is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. A “recombinant” protein is encoded by a recombinant polynucleotide. In the context of a genetically modified host cell, a “recombinant” host cell refers to both the original cell and its progeny.

As used herein, the term “genetically modified” refers to any change in the endogenous genome of a cyanobacteria cell compared to a wild-type cell. Thus, changes that are introduced through recombinant DNA technology and/or classical mutagenesis techniques are both encompassed by this term. The changes may involve protein coding sequences or non-protein coding sequences such as regulatory sequences as promoters or enhancers.

An “expression construct” or “expression cassette” as used herein refers to a recombinant nucleic acid construct, which, when introduced into a cyanobacterial host cell in accordance with the present invention, results in increased expression of a fusion protein encoded by the nucleic acid construct. The expression construct may comprise a promoter sequence operably linked to a nucleic acid sequence encoding the fusion protein or the expression cassette may comprise the nucleic acid sequence encoding the fusion protein where the construct is configured to be inserted into a location in a cyanobacterial genome such that a promoter endogenous to the cyanobacterial host cell is employed to drive expression of the fusion protein. An “expression unit” as used herein refers to a minimal region of a polynucleotide that is expressed that provided for high level protein expression, which comprises the polynucleotide that encodes the fusion protein, as well as other genes. e.g., cpcA and cpc operon genes encoding cpc linker polypeptides CpcC2, CpcC1, and CpcD. In some embodiments, the expression unit additionally include a gene encoding an antibiotic resistance polypeptide, such as a chloramphenicol resistance gene or streptomycin resistance gene. The expression unit may also comprise additional sequences, such as nucleic acid sequences encoding a protease cleavage site, a spacer polypeptide, or a polypeptide tagging sequence, such as a His tag. As used herein, “expression” and “overexpression” are used interchangeably to refer to expression of a fusion protein in the host cell.

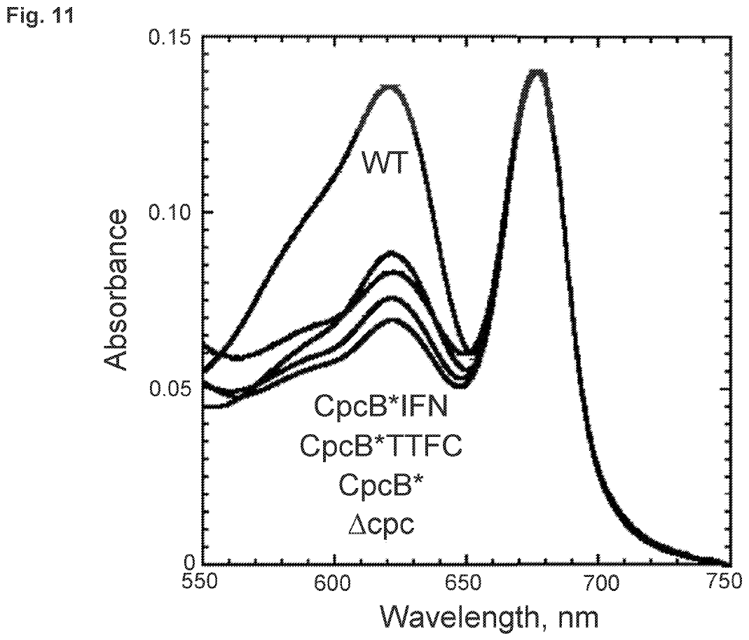

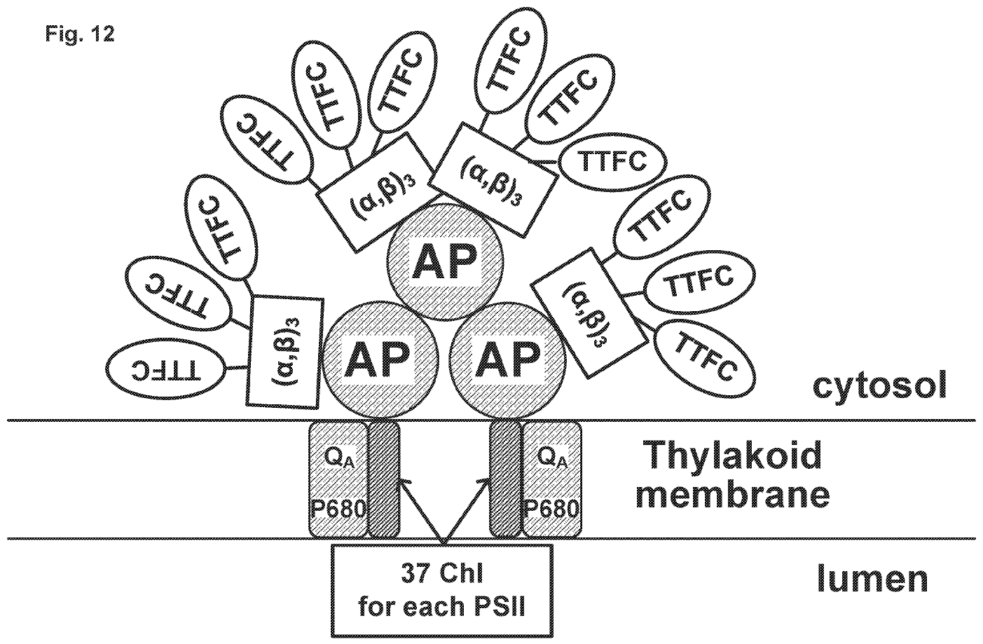

As used herein, “heterohexameric disc” or “hexameric disc” are used interchangeably to refer to a disc structure that is componed of three CpcA α- and three CpcB β-phycocyanin subunits. Recombinantly fused proteins, fused to CpcB and/or CpcA, emanate radially from the heterohexameric disc. The linker CpcG protein occupies the disc center. Not to be bound by theory, but the heterologous fusion protein is thought to be distal to the heterohexameric compact disc (FIG. 10), i.e., it is exposed to the aqueous cytosolic medium so that it does not interfere with the anchoring of the heterohexamer onto the cyanobacterial AP core cylinders (FIG. 12). A protein of interest may also be fused to the linker protein CpcG such that it does not interefere with the noted heterohexameric disc properties.

By “construct” is meant a recombinant nucleic acid, generally recombinant DNA. which has been generated for the purpose of the expression of a specific nucleotide sequence(s), or is to be used in the construction of other recombinant nucleotide sequences.

As used herein, the term “exogenous protein” refers to a protein that is not normally or naturally found in and/or produced by a given cyanobacterium, organism, or cell in nature. As used herein, the term “endogenous protein” refers to a protein that is normally found in and/or produced by a given cyanobacterium, organism, or cell in nature.

An “endogenous” protein or “endogenous” nucleic acid is also referred to as a “native” protein or nucleic acid that is found in a cell or organism in nature.

The terms “nucleic acid” and “polynucleotide” are used synonymously and refer to a single or double-stranded polymer of deoxyribonucleotide or ribonucleotide bases read from the 5′ to the 3′ end. A nucleic acid of the present invention will generally contain phosphodiester bonds, although in some cases, nucleic acid analogs may be used that may have alternate backbones, comprising, e.g., phosphoramidate, phosphorothioate, phosphorodithioate, or O-methylphophoroamidite linkages (see Eckstein, Oligonucleotides and Analogues: A Practical Approach. Oxford University Press); and peptide nucleic acid backbones and linkages. Other analog nucleic acids include those with positive backbones; non-ionic backbones, and non-ribose backbones. Thus, nucleic acids or polynucleotides may also include modified nucleotides, that permit correct read through by a polymerase. “Polynucleotide sequence” or “nucleic acid sequence” may include both the sense and antisense strands of a nucleic acid as either individual single strands or in a duplex. As will be appreciated by those in the art, the depiction of a single strand also defines the sequence of the complementary strand: thus the sequences described herein also provide the complement of the sequence. Unless otherwise indicated, a particular nucleic acid sequence also implicitly encompasses conservatively modified variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc.

The term “promoter” or “regulatory element” refers to a region or sequence determinants located upstream or downstream from the start of transcription that are involved in recognition and binding of RNA polymerase and other proteins to initiate transcription. A “cyanobacteria promoter” is a promoter capable of initiating transcription in cyanobacteria cells. Such promoters need not be of cyanobacterial origin, for example, promoters derived from other bacteria or plant viruses, can be used in the present invention.

Two nucleic acid sequences or polypeptides are said to be “identical” if the sequence of nucleotides or amino acid residues, respectively, in the two sequences is the same when aligned for maximum correspondence as described below. The term “complementary to” is used herein to mean that the sequence is complementary to all or a portion of a reference polynucleotide sequence.

Optimal alignment of sequences for comparison may be conducted by the local homology algorithm of Smith and Waterman Add. APL. Math. 2:482 (1981), by the homology alignment algorithm of Needle man and Wunsch J. Mol. Biol. 48:443 (1970), by the search for similarity method of Pearson and Lipman Proc. Natl. Acad. Sci. (U.S.A.) 85:2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by inspection.

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” in the context of polynucleotide or polypeptide sequences means that a polynucleotide or polypeptide comprises a sequence that has at least 50% sequence identity to a reference nucleic acid or polypeptide sequence. Alternatively, percent identity can be any integer from 40% to 100%. Exemplary embodiments include at least: 50%, 55%, 60%, 65%. 70%, 75%, 80%, 85%, 90%, 95%, or 99% compared to a reference sequence using the programs described herein; preferably BLAST using standard parameters, as described below.

Another indication that nucleotide sequences are substantially identical is if two molecules hybridize to each other, or a third nucleic acid, under stringent conditions. Stringent conditions are sequence dependent and will be different in different circumstances. Generally, stringent conditions are selected to be about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is about 0.02 molar at pH 7 and the temperature is at least about 60° C.

The term “isolated”, when applied to a nucleic acid or protein, denotes that the nucleic acid or protein is essentially free of other cellular components with which it is associated in the natural state. It is preferably in a homogeneous state and may be in either a dry or aqueous solution. Purity and homogeneity are typically determined using analytical chemistry techniques such as polyacrylamide gel electrophoresis or high-performance liquid chromatography. A protein which is the predominant species present in a preparation is substantially purified. In particular, an isolated gene is separated from open reading frames which flank the gene and encode a protein other than the gene of interest.

### Expression of a Hexameric Complex

The present disclosure is based, at least in part, on the discovery of the structure of proteins expressed from fusion protein constructs in which the protein to be expressed, typically a non-native protein not expressed in cyanobacteria, is fused at the C-terminus of a cyanobacteria CpcB polypeptide. In some embodiments, the protein is fused at the N-terminus of the CpcB polypeptide. In other embodiments, a second polypeptide to be expressed in cyanobacteria, which may be the same non-native protein or a different polypeptide, is fused to the C-terminus of a CpcA polypeptide. In some embodiments, the second polypeptide is fused to the N-terminus of a CpcA polypeptide. Thus, in accordance with some aspects of the present disclosure, engineering of a cyanobacterial cell results in an expression unit in the cyanobacterial cell comprising i) a nucleic acid sequence comprising the transgene, wherein the transgene is fused to the 3′ end of a nucleic acid sequence that encodes a cyanobacteria B-subunit of phycocyanin (CpcB) polypeptide, or to the 5′ end of a nucleic acid sequence that encode the CpcB polypeptide, to produce a fusion polypeptide comprising CpcB and the protein of interest: (ii) a nucleic acid sequence encoding a cyanobacteria α-subunit of phycocyanin (CpcA) polypeptide, which may or may not be fused, at the C-terminal end or N-terminal end, to a second protein of interest to be expressed in the cyanobacterial cell; and (iii) a nucleic acid sequence encoding a cyanobacterial CpcG polypeptide. The expression unit expresses a complex comprising the protein of interest as a component of a hexameric disc complex comprising a cyanobacterial CpcA phycocyanin subunit protein fused, or not fused to a second protein of interest, a CpcB fusion protein, and CpcG is a phycocyanin linker polypeptide. Formation of the hexameric complex comprising the protein of interest results in high levels of accumulation of the protein encoded by the transgene.

The disclosure additionally provides nucleic acids encoding a fusion protein as described herein, as well as expression constructs comprising the nucleic acids and host cells that have been genetically modified to express such fusion proteins. In further aspects, the disclosure provides methods of producing the hevameric discs comprising one or more proteins of interest to be expressed and in some embodiments, products generated by the proteins using such genetically modified cyanobacterial cells. In some embodiments, the method comprises isolating a hexameric complex comprising the fusion polypeptide, wherein the hexameric disc complex is at least 90% (w/w) at least 95% (w/w), or at least 99% (w/w) pure.

The invention employs various routine recombinant nucleic acid techniques. Generally, the nomenclature and the laboratory procedures in recombinant DNA technology described below are those commonly employed in the art. Many manuals that provide direction for performing recombinant DNA manipulations are available, e.g., Sambrook, Molecular Cloning, A Laboratory Manual (4th Ed, 2012); and Current Protocols in Molecular Biology (Ausubel et al., eds., 1994-2021).

### CpcB and CpcA Fusion Polypeptides

In the present disclosure, a transgene encoding a protein of interest is joined at the 3′ end, or that the 5′ end, of a CpcB polypeptide to express a fusion protein in which the protein of interest is fused to the carboxyl end, or the N-terminal end, respectively of a CpcB polypeptide. In some embodiments, a second transgene encoding a second protein of interest is joined to a nucleic acid sequence encoding a CpcA polypeptide to express a second fusion protein in which the second protein of interest is fused to the carboxyl-terminal or N-terminal end of CpcA. In some embodiments, the second protein is the same protein that is fused to CpcB. In other embodiments, the second polypeptide is a different protein. As illustrated herein, the first and second protein of interest need not be fused directly to the CpcB or CpcA protein, but can be separated by other sequences, e.g., spacers, purification tags, and/or protease cleavage sites.

In some embodiments, the CpcB sequence or CpcA sequence encodes less than the full-length of the protein, but typically comprises a region that encodes at least 80%, or at least 90%, or at least 95%, or greater, of the length of the protein. As appreciated by one of skill in the art, use of an endogenous CpcB or CpcA cyanobacterial polynucleotide sequence for constructing an expression construct in accordance with the invention provides a sequence that need not be codon-optimized, as the sequence is already expressed at high levels in cyanobacteria. Examples of cyanobacterial polynucleotides that encode CpcB and CpcA are available at the website www.genome.microbedb.jp/cyanobase under accession numbers, as follows:
cpcA: Synechocystis sp. PCC 6803 sll1578, Anabaena sp. PCC 7120 arl0529, Thermosynechococcus elongatus BP-1 tlr1958, Synechococcus elongatus PCC 6301 syc0495_c, syc0500_c
cpcB: Synechocystis sp. PCC 6803 sll1577, Anabaena sp. PCC 7120 ar10528, Thermosynechococcus elongatus BP-1 tlr1957, Synechococcus elongatus PCC 6301 syc0496_c, sye0501_c.
cpcG: Synechocystis sp. PCC 6803 sll1471 slr2051; Anabaena sp. PCC 7120 alr0534, alr0535, alr0536, alr0537; Thermosynechococcus elongatus BP-1 dlr1963, tlr1964, tlr 1965; Synechococcus elongatus PCC 6301 syc2065_d.
cpcC1: Synechocystis sp. PCC 6803 sll1580, Anabaena sp. PCC 7120 alr0530, Thermosynechococcus elongatus BP-1 tlr1959, Synechococcus elongatus PCC 6301 syc0498_c

In some embodiments, the polynucleotide sequence that encodes the cpcA or cpcB protein need not be 100% identical to the native cyanobacteria polynucleotide sequence. A polynucleotide variant having at least 60%, at least 65%, at or at least 70% or greater, identity to a native cyanobacterial polynucleotide sequence, e.g., a native cpcB or cpcA cyanobacteria polynucleotide sequence, may also be used, so long as the codons that vary relative to the native cyanobacterial polynucleotide are codon optimized for expression in cyanobacteria and the codons that vary relative to the wild type sequence do not substantially disrupt the structure of the protein. In some embodiments, a polynucleotide variant that has at least 75% identity, at least 80% identity, or at least 85% identity, or greater to a native cyanobacterial polynucleotide sequence, e.g., a native cpcB or cpcA cyanobacteria polynucleotide sequence, is used, again maintaining codon optimization for cyanobacteria, as desired. In some embodiments, a polynucleotide variant that has least 90% identity, or at least 95% identity, or greater, to a native cyanobacterial polynucleotide sequence, e.g., a native cpcB or cpcA cyanobacteria polynucleotide sequence, is used. The percent identity is typically determined with reference the length of the polynucleotide that is employed in the construct, i.e., the percent identity may be over the full length of a polynucleotide that encodes the leader polypeptide sequence, or may be over a smaller length, e.g., in embodiments where the polynucleotide encodes at least 75%, or at least 90%, or at least 95%, or greater, of the length of the protein. A codon that varies from the wild-type polynucleotide is typically selected such that the protein structure of the native cyanobacterial sequence is not substantially altered by the changed codon, e.g., a codon that encodes an amino acid that has the same charge, polarity, and/or is similar in size to the native amino acid is selected. In some embodiments, the CpcA or CpcB polypeptide encoded by the nucleic acid has at least 90% or at least 95% identity to a antive cyanobacteria CpcA or CpcB polypeptide, e.g., a native Synechocystis sp. PCC6803 sll1578, Anabaena sp. PCC7120 ar10529, Thermosynechococcus elongatus BP-1 tlr1958, or Synechococcus elongates sequence.

### CpcG Linker

In some embodiments, a fusion construct of the present disclosure is expressed in a configuration that results in a structure (α,β*P)₃CpcG, where a is a cyanobacterial CpcA phycocyanin subunit protein, β is a cyanobacterial CpcB phycocyanin subunit protein, the asterisk denotes fusion, P is the protein of interest in the fusion construct, and CpcG is a phycocyanin linker polypeptide. In some embodiments, the protein P is fused to CpcB or CpcA at the carboxyl end of the CpcB or CpcA polypeptide. In some embodiments, the protein P is fused to CpcB or CpcA at the amino terminal end of the CpcB or CpcA polypeptide. The phycocyanin-associated linker proteins CpcG, CpcC1, CpcC2 and CpcD participate in connecting the (α,β)₃ discs to one another and to the core cylinders, thereby facilitating the formation of a functional stack of multiple discs comprising the phycocyanin rods. More specifically, the CpcG linkers participate in linking the first (α,β)₃ phycocyanin disc to the allophycocyabnin phycobilisome core cylinders. CpcC1 helps to secure phycocyanin disc #2 onto disc #1, and CpcC2 helps to secure phycocyanin disc #3 onto disc #2. CpcD is located immediately following the C-terminus of the CpcC2 linker, potentially acting as a terminal rod growth indicator, helping to ensure uniform rod length among the multiple phycocyanin rods of the phycobilisome (de Lorimier et al. 1990).

The configuration of the fusion construct is based on the formation of a (α,β*Protein)₃CpcG heterohexamer complex as disclosed herein, which acts as a light-harvesting antenna by a host cyanobacterial cell. Not to be bound by theory, it is thought that the complex is retained and accumulates in the host cell, which tolerates the presence of the heterologous recombinant proteins as fusions, so long as they are placed in a radial position with respect to the (α,β)₃CpcG heterohexamer and do not interfere with the binding and function of the heterohexameric complex to the core allophycocyanin cylinders of the cyanobacterial phycobilisome.

In some embodiments, a fusion construct configuration is employed such that based on the structure described herein, positions the recombinant fusion protein radially in relation to the (α,β)₃CpcG heterohexamer disc: (α,β*P)₃CpcG, in which the protein of interest is fused to the carboxy-terminal end of CpcB; (α,P*β)₃CpcG, in which the protein of interest is fused to the amino-terminal end of CpcB; (α*P,β)₃CpcG, in which the protein of interest is fused to the carboxy-terminal end of CpcA; or

(P*α,β)₃CpcG, in which the protein of interest is fused to the amino-terminal end of CpcA.

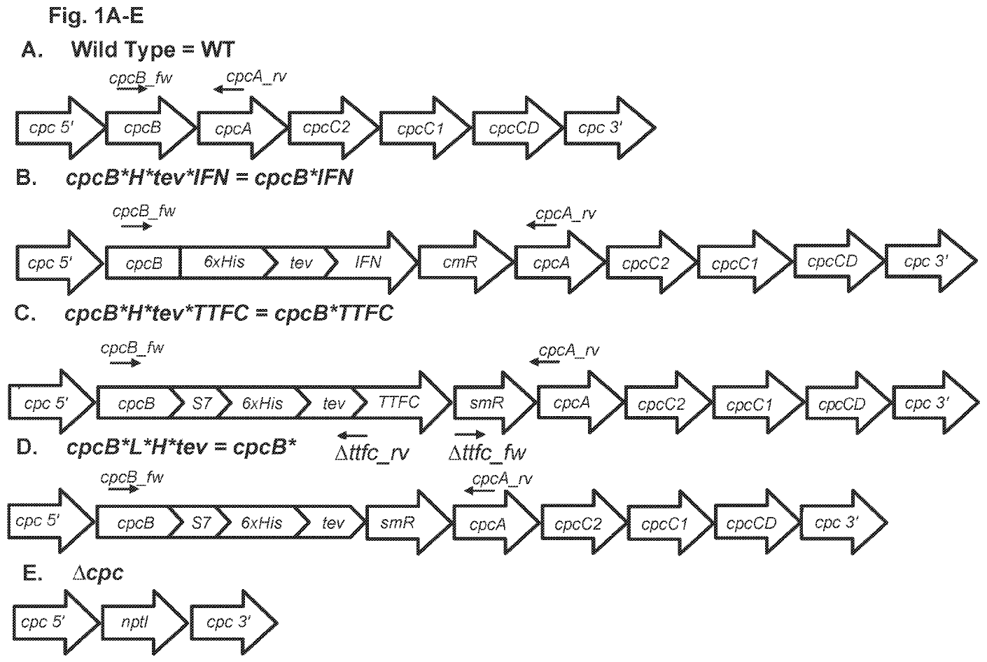

In typical embodiments, a suitable spacer polypeptide (see, e.g., FIG. 1C; and Chaves et al. 2017) is placed between the CpcB and recombinant protein, or between CpcA and recombinant protein in the fusion construct. A variety of suitable spacers can be employed and include the following:
PMPWRVI, a spacer comprising 7 amino acids (S7), which as used in this disclosure, is designed to separate the two proteins based on the secondary structure of the spacer and to change by about 90 degrees in the proline position the relative orientation of the leading as compared to the trailing protein in a fusion construct;
(PA)₅, a spacer comprising 10 amino acids (S10), which includes five alternating segments of proline and alanine. The small size of the proline, the cyclic side chain, and the lack of free amino or carboxy groups may prevent interactions with other amino acids in the main protein domains, and cause a very restricted, or rigid spacer structure.
EAAAKEAAAKEAAAKA, comprising repeat EAAAK segments, and forming a rigid, straight, and stable α-helix spacer; and
PWRVICATSSQFTQITEHNSRRSANYQPNLWNFEFLQSLENDLKVEKLEEKA TKLEEEVRPWRVI, which repeats the first 65 amino acids of the isoprene synthase enzyme, used as a method to duplicate a portion of the enzyme with important catalytic activity (Chaves et al. 2017).

### CpcC1 Linker

In some embodiments, a fusion construct is expressed to provide an alternative configuration, e.g., a (α,β*P)₃CpcC1 structure (i.e., a heterohexamer disc in which CpC1 serves as a linker). Not to be bound by theory, identification of the CpcC1 linker in purified fractions as described in the examples indicated formation and assembly of the middle phycocyanin (α,β*P)₃CpcC1 heterohexamer disc, which is expected to stack behind the (α,β)₃CpcG heterohexamer disc, a step further away from the AP core. The additional (α,β*P)₃CpcC1 heterohexamer disc thus assembles in the fusion construct configuration in the transformants, allowing for expression of additional proteins in the following fusion constructs configuration:
(α,β*P)₃CpcC1
(α,P*β)₃CpcC1
(α*P,β)₃CpcC1
(P*α,β)₃CpcC1

In such fusion constructs, heterohexamenr discs are present in addition to those stabilized by the CpcG linker proteins and thus afford the formation of a higher-order structure in which the (α,β*P)₃CpcG-based fusion constructs are proximal to the allophycocyanin core cylinders, whereas (α,β*P)₃CpcC1-based fusion constructs form a functional light-harvesting disc stacked on top of the (α,β)₃CpcG heterohexamer disc, paralleling the natural configuration of the phycocyanin discs in the cyanobacterial phycobilisome.

### CpcG Fusion Proteins

In some embodiments, a hexameric disc structure as described herein comprising CpcA, CpcB, and CpcG comprises a fusion protein in which a protein of interest is fused to CpcG. In some embodiments, the protein of interest is fused to the N-terminal end of CpcG. In other embodiments, the protein of interest is fused to the C-terminal end of CpcG. In some embodiments, such a hexameric disc structure comprises CpcB and/or CpcA fusion proteins as described herein. The CpcB and/or CpcA fusion protein may comprise the same protein of interest that is contained in the CpcG fusion protein or in some embodiments, may comprise a different protein of interest. As illustrated herein, a protein of intereset need not be fused directly to CpcG, but can be separated by other sequences, e.g., spacers, purification tags, and/or protease cleavage sites. In some embodiments, a protein of interest is fused to CpcG and a second protein of interest is fused to CpcA or CpcB.

### CpcC1 Fusion Proteins

In some embodiments, a hexameric disc structure as described herein comprising

CpcA, CpcB, and CpcC1 comprises a fusion protein in which a protein of interest is fused to CpcC1. In some embodiments, the protein of interest is fused to the N-terminal end of CpcC1. In other embodiments, the protein of interest is fused to the C-terminal end of CpcC1. In some embodiments, such a hexameric disc structure comprises CpcB and/or CpcA fusion proteins as described herein. The CpcB and/or CpcA fusion protein may comprise the same protein of interest that is contained in the CpcC1 fusion protein or in some embodiments, may comprise a different protein of interest.

### Transgenes

A fusion construct of the invention may be employed to provide high level expression in cyanobacteria for any desired protein. Thus, for example, cyanobacteria can be engineered to express an animal biopharmaceutical polypeptide such as an antibody, hormone, cytokine, therapeutic enzyme and the like, as a fusion polypeptide with a protein expressed at a high level in cyanobacteria, e.g. a CpcB or other protein encoded by the cpc operon. In some embodiments the biopharmaceutical polypeptide is expressed at a level of at least 1%, or at least 5%, or at least 10%, or at least 15%, or at least 20%, of total cellular protein as described herein. In some embodiments, a protein of interest is G-CSF, GM-CSF, MCP1 sCD40L, TGF-alpha, EGF, FGF-2, Flt-3L, INF-apha2, INF-gamma, IL-10, IL-15, IL-17, IL-1beta, IL-2, IL-6, IL-8, IL-10, IL-15, IL-17, IL-1beta, IL-2, IL-6, IL-8, IP-10, MIP-1beta, PDGF-AA, TNF-alpha, or VEGF. In some embodiments, cyanobacteria are engineered to produce a desired product such as isoprene, hemiterpene; beta-phellandrene, a monoterpene; farnesene, a sesquiterpene; or other products. Accordingly, proteins such as isoprene synthase, beta-phellandrene synthase form a variety of plants, geranyl diphosphate synthase and geranyl linalool synthase can be produced. See also WO2017205788, and WO2016210154, each incorporated by reference for proteins that can be expressed in cyanobacteria in order to obtain a product. This listing of proteins is not intended to be comprehensive, as the method can be used to express any number of proteins.

In some embodiments, the nucleic acid sequence encoding the polypeptide to be expressed, e.g., a plant or animal polypeptide, is codon-optimized for expression in cyanobacteria. Alternatively, the nucleic acid sequence need not be codon-optimized, as high-level expression of the fusion polypeptide does not require codon optimization.

In some embodiments, the mature form of a polypeptide lacking the native signal sequence is expressed.

In some embodiments, the transgene that is expressed encodes an interferon, e.g., an interferon alpha, such as human interferon alpha, or other cytokine. An illustrative interferon polypeptide sequence is available under uniprot number P01563. The amino acid sequence of a mature form of human interferon alpha-2 is shown in the sequences provided at the end of the Examples section. In some embodiments, the IFNA2 protein is expressed as a fusion construct with cpcB, e.g., by replacing the cpcB gene in the cpc operon with a transgene encoding a cpcB*interferon fusion construct. In some embodiments, a protein of interest is G-CSF, GM-CSF, MCPI sCD40L, TGF-alpha, EGF, FGF-2, Flt-3L, INF-apha2, INF-gamma, IL-10, IL-15, IL-17, IL-1beta, IL-2, IL-6, IL-8, IL-10, IL-15, IL-17, IL-1beta, IL-2, IL-6, IL-8, IP-10, MIP-1beta, PDGF-AA, TNF-alpha, or VEGF.

In some embodiments, the transgene that is expressed encodies tetanus toxin fragment C (TTFC). The amino acid sequence of an illustrative TTFC polypeptide is shown in the sequences provided at the end of the Examples section. In some embodiments, the TTFC protein is expressed as a fusion construct with cpcB, e.g., by replacing the cpcB gene in the cpc operon with a transgene encoding a cpcB*interferon fusion construct.

In some embodiments, e.g., when an expressed protein product is to be purified, the fusion polypeptide comprises a protease cleavage site such as a Factor Xa cleavage site or alternative cleavage site, e.g., a Tobacco Etch Virus (TEV) cysteine protease cleavage site. Alternatively, the fusion polypeptide may comprise an Enteropeptidase, Thrombin, Protease 3C, Sortase A, Genase I, Intein, or a Snac-tag cleavage site (e.g., Kosobokova et al. 2016; Dang et al. 2019). In some embodiments, the fusion polypeptide may comprise a protein purification tag, such as a 6XHis tag.

As noted above, in some embodiments, the transgene portion of a fusion construct in accordance with the invention may be, but is not required to be, codon optimized for expression in cyanobacteria. For example, in some embodiments, codon optimization is performed such that codons used with an average frequency of less than 12% by Synechocystis are replaced by more frequently used codons. Rare codons can be defined, e.g., by using a codon usage table derived from the sequenced genome of the host cyanobacterial cell. See, e.g., the codon usage table obtained from Kazusa DNA Research Institute, Japan (website www.kazusa.or.jp/codon/) used in conjunction with software, e.g., “Gene Designer 2.0” software, from DNA 2.0 (website www.dna20.com/) at a cut-off thread of 15%; or the software available at the website, idtdna.com/CodonOpt.

### Preparation of Recombinant Expression Constructs

Recombinant DNA vectors suitable for transformation of cyanobacteria cells are employed in the methods of the invention. Preparation of suitable vectors and transformation methods can be prepared using any number of techniques, including those described, e.g., in Sambrook, Molecular Cloning, A Laboratory Manual (4th Ed, 2012); and Current Protocols in Molecular Biology (Ausubel et al., eds., 1994-2015). For example, a DNA sequence encoding a fusion protein of the present invention will be combined with transcriptional and other regulatory sequences to direct expression in cyanobacteria.

In some embodiments, the vector includes sequences for homologous recombination to insert the fusion construct at a desired site in a cyanobacterial genome, e.g., such that expression of the polynucleotide encoding the fusion construct will be driven by a promoter that is endogenous to the organism. A vector to perform homologous recombination will include sequences required for homologous recombination, such as flanking sequences that share homology with the target site for promoting homologous recombination.

Regulatory sequences incorporated into vectors that comprise sequences that are to be expressed in the modified cyanobacterial cell include promoters, which may be either constitutive or inducible. In some embodiments, a promoter for a nucleic acid construct is a constitutive promoter. Examples of constitutive strong promoters for use in cyanobacteria include, for example, the psbD1 gene or the basal promoter of the psbD2 gene, or the rbcLS promoter, which is constitutive under standard growth conditions. Various other promoters that are active in cyanobacteria are also known. These include the strong cpc operon promoter, the cpc operon and ape operon promoters, which control expression of phycobilisome constituents. The light inducible promoters of the psbA1, psbA2, and psbA3genes in cyanobacteria may also be used, as noted below. Other promoters that are operative in plants, e.g., promoters derived from plant viruses, such as the CaMV35S promoters, or bacterial viruses, such as the T7, or bacterial promoters, such as the PTre, can also be employed in cyanobacteria. For a description of strong and regulated promoters, e.g., active in the cyanobacterium Anabaena sp. strain PCC 7120 and Synechocystis 6803, see e.g., Elhai, FEMS Microbiol Lett 114:179-184, (1993) and Formighieri, Planta 240:309-324 (2014).

In some embodiments, a promoter can be used to direct expression of the inserted nucleic acids under the influence of changing environmental conditions. Examples of environmental conditions that may affect transcription by inducible promoters include anaerobic conditions, elevated temperature, or the presence of light. Promoters that are inducible upon exposure to chemicals reagents are also used to express the inserted nucleic acids. Other useful inducible regulatory elements include copper-inducible regulatory elements (Mett et al., Proc. Natl. Acad. Sci. USA 90:4567-4571 (1993): Furst et al.., Cell 55:705-717 (1988)); copper-repressed petJ promoter in Synechocystis (Kuchmina et al. 2012, J Biotechn 162:75-80); riboswitches, e.g. theophylline-dependent (Nakahira et al. 2013, Plant Cell Physiol 54:1724-1735; tetracycline and chlor-tetracycline-inducible regulatory elements (Gatz et al., Plant J. 2:397-404 (1992); Röder et al., Mol. Gen. Genet. 243:32-38 (1994); Gatz, Meth. Cell Biol. 50:411-424 (1995)); ecdysone inducible regulatory elements (Christopherson et al., Proc. Natl. Acad. Sci. USA 89:6314-6318 (1992); Kreutzweiser et al., Ecotoxicol. Environ. Safety 28:14-24 (1994)); heat shock inducible promoters, such as those of the hsp70/dnak genes (Takahashi et al., Plant Physiol. 99:383-390 (1992); Yabe et al., Plant Cell Physiol. 35:1207-1219 (1994); Ueda et al., Mol. Gen. Genet. 250:533-539 (1996)); and lac operon elements, which are used in combination with a constitutively expressed lac repressor to confer, for example, IPTG-inducible expression (Wilde et al., EMBO J. 11:1251-1259 (1992)). An inducible regulatory element also can be, for example, a nitrate-inducible promoter, e.g., derived from the spinach nitrite reductase gene (Back et al., Plant Mol. Biol. 17:9 (1991)), or a light-inducible promoter, such as that associated with the small subunit of RuBP carboxylase or the LHCP gene families (Feinbaum et al., Mol. Gen. Genet. 226:449 (1991); Lam and Chua, Science 248:471 (1990)).

In some embodiments, the promoter may be from a gene associated with photosynthesis in the species to be transformed or another species. For example, such a promoter from one species may be used to direct expression of a protein in transformed cyanobacteria cells. Suitable promoters may be isolated from or synthesized based on known sequences from other photosynthetic organisms. Preferred promoters are those for genes from other photosynthetic species, or other photosynthetic organism where the promoter is active in cyanobacteria.

A vector will also typically comprise a marker gene that confers a selectable phenotype on cyanobacteria transformed with the vector. Such marker genes, include, but are not limited to those that confer antibiotic resistance, such as resistance to chloramphenicol, kanamycin, spectinomycin, G418, bleomycin, hygromycin, and the like.

Cell transformation methods and selectable markers for cyanobacteria are well known in the art (Wirth, Mol. Gen. Genet., 216 (1): 175-7 (1989); Koksharova, Appl. Microbiol. Biotechnol., 58 (2): 123-37 (2002); Thelwell et al., Proc. Natl. Acad. Sci. U.S.A., 95:10728-10733 (1998)).

In some embodiments, a gene editing technique, such as a CRISPR/Cas. TALENS, or zinc finger nuclease technique, is employed to introduce a nucleic acid sequence encoding a fusion protein into the cpc operon at one or more locations, e.g., in the cpcB locus, cpcC locus, and/or cpcG locus, of a cyanobacterial genome for expression.

Any suitable cyanobacteria may be employed to express a fusion protein in accordance with the invention. These include unicellular cyanobacteria, micro-colonial cyanobacteria that form small colonies, and filamentous cyanobacteria. Examples of unicellular cyanobacteria for use in the invention include, but are not limited to, Synechococcus and Thermosynechococcus sp., e.g., Synechococcus sp. PCC 7002, Synechococcus sp. PCC 6301, and Thermosynechococcus elongatus; as well as Synechocystis sp., such as Synechocystis sp. PCC 6803: and Cyanothece sp., such as PCC 8801. Examples of micro-colonial cyanobacteria for use in the invention, include, but are not limited to, Gloeocapsa magma, Gloeocapsa phylum, Gloeocapsa alpicola, Gloeocpasa atrata, Chroococcus spp., and Aphanothece sp. Examples of filamentous cyanobacteria that can be used include, but are not limited to, Oscillatoria spp.., Nostoc sp., e.g., Nostoc sp. PCC 7120, and Nostoc sphaeroides; Anabaena sp., e.g., Anabaena variabilis and Arthrospira sp., such as Arthrospira platensis and Arthrospira maxima, and Mastigocladus laminosus. Some Arthrospira sp., e.g., Arthrospira platensis, Arthrospira fusiformis, and Arthrospira maxima have also been referred to as species of Spirulina. Cyanobacteria that are genetically modified in accordance with the invention may also contain other genetic modifications, e.g., modifications to the terpenoid pathway, to enhance production of a desired compound.

Cyanobacteria can be cultured to high density, e.g., in a photobioreactor (see, e.g., Lee et al., Biotech. Bioengineering 44:1161-1167, 1994; Chaumont, J Appl. Phycology 5:593-604, 1990) to produce the protein encoded by the transgene. In some embodiments, the protein product of the transgene is purified. In many embodiments, the cyanobacteria culture is used to produce a desired, non-protein product, e.g., isoprene, a hemiterpene; β-phellandrene, a monoterpene; farnesene, a sesquiterpene: or other products. The product produced from the cyanobacteria may then be isolated or collected from the cyanobacterial cell culture.

### Purification of Protein

A hexameric disc complex expressed in cyanobacteria modified as described herein can be purified using known techniques, e.g., by incorporating a His tag or an alternative purification tag into the expressed proteins to be used for affinity purification. In some embodiments, the purified hexameric disc preparation is at least 90% (w/w), or at least 95% (w/v) pure. In some embodiments, the purified hexameric disc preparation is at least 99% (w/w) pure.

In some embodiments, a protein of interest encoded by a fusion protein may be cleaved from the hexameric disc following an affinity chromatography step. In some embodiments, a protein of interest can be separated from the fusion protein via protease cleavage at a cleavage site present in the fusion protein and the protein of interest purified using know purification procedures. Thus, for example, in some embodiments, an enzyme or biopharmaceutical protein, e.g., a cytokine, poypeptide hormone or other protein of interest, may be cleaved to provide a purified preparation. In some embodiments, a protein of interest is G-CSF, GM-CSF, MCP1 sCD40L, TGF-alpha, EGF, FGF-2, Flt-3L, INF-apha2, INF-gamma, IL-10, IL-15, IL-17, IL-1beta, IL-2, IL-6, IL-8, IL-10, IL-15, IL-17, IL-1beta, IL-2, IL-6, IL-8, IP-10, MIP-1beta, PDGF-AA, TNF-alpha, or VEGF.

In some embodiments, a hexameric disc preparation, comprising one or more proteins of interest expressed in cyanobacteria is used in an immunoassay, e.g., for diagnostic applications. In some embodiments, a hexameric disc comprising a protein of interest may be used in an oral vaccine preparation. In some embodiments, the hexameric disc preparation is at least 95% (w/w) or at least 99% (w/w) pure.

### Detailed Technical Description of Illustrative Embodiments

The following descriptions of fusion protein constructs and protein production

provide illustrative embodiments of high-level expression of exogenous polypeptides, such as biopharmaceutical proteins, in cyanobacteria.

### 1. Expression of an Interferon and TTFC in Cyanobacteria

This example demonstrates the structure of a fusion protein comprising human interferon α-2 protein (Uniprot No. P01563), referred to in this example as IFN; and a fusion protein comprising tetanus toxin fragment C (TTFC) in the cyanobacteria Synechocystis sp. PCC 6803 (Synechocystis).

Expression of IFN and TTFC is accomplished by genetic engineering of the cpc operon which, in the wild type, encodes the light-harvesting phycocyanin CpcB (β-) and CpcA (α-) subunits and their associated CpcC2, CpcC1, and CpcD linker polypeptides. The various genetic configurations of the cpc operon with the heterologous genes employed in this example are shown in FIG. 1. The wild type (WT) cpc operon in Synechocystis is shown in FIG. 1A. Fusion construct cpcB*6xHis*tev*IFN (abbreviated as CpcB*IFN) is the transformant whereby the human interferon α-2 encoding gene is fused to the cpcB gene through the six histidine and tobacco etch virus tev protease cleavage domain encoding DNA sequences (FIG. 1B). The 6xHis was designed to enable isolation through affinity chromatography of the fusion protein, whereas the tev would allow cleavage of the IFN protein from the leader CpcB*6xHis polypeptide (Zhang et al. 2021). The chloramphenicol (cmR) resistance cassette for transformant selection was also included in an operon configuration downstream of the fusion DNA (Betterle et al. 2020).

In similar fashion, cpcB*6xHis*tev*TTFC (abbreviated as CpcB*TTFC) is the transformant containing the tetanus toxin fragment C encoding gene, followed by a spectinomycin resistance cassette (FIG. 1C, Zhang et al. 2021). A new cpcB*6xHis*tev transformant (referred to simply as CpcB*, FIG. 1D) is a variant of the CpcB*TTFC construct, and it includes only the fusion of the tev cleavage and histidine encoding sequences to the cpcB gene, followed by the spectinomycin resistance cassette. Lastly, the Δcpc transformant is lacking the entire cpc operon, as this was deleted upon replacement with the kanamycin resistance cassette (FIG. 1E, Kirst et al. 2014).

Genomic DNA PCR analysis was employed to test for fusion construct locus insertion and attainment of homoplasmy in the above transformant strains. Primers cpcB_fw and cpcA_rv were used, overlapping the cpcB and cpcA genes, respectively (FIG. 1). Primer sequences were as follows:

cpcB_fw
5′-TGACATGGAAATCATCCTCC-3′
cpcA_rv
5′-GGTGGAAACGGCTTCAGTTAAAG-3′
Attfc_fw
5′-TGAGGAATTAGGAGGTAATATATG-3′
Attfc rv
5′-GCCTTGTAAATACAAATTATCATG-3′.

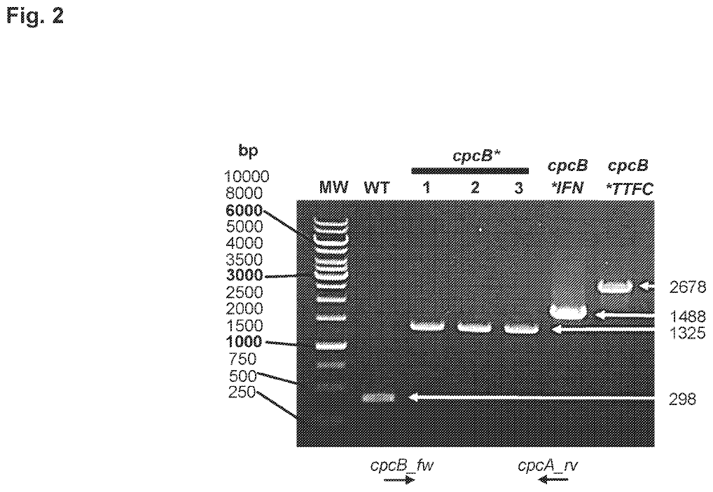

Results from this PCR analysis include the WT amplicon of 298 bp, CpcB* amplicon of 1325 bp, CpcB*IFN amplicon of 1488 bp, and CpcB*TTFC amplicon of 2678 bp (FIG. 2). No PCR products were detected with the Δcpc transformant DNA (not shown). Absence of wild type products in any of the transformants examined showed complete segregation of the transformant DNA and attainment of homoplasmy in the transgenic strains.

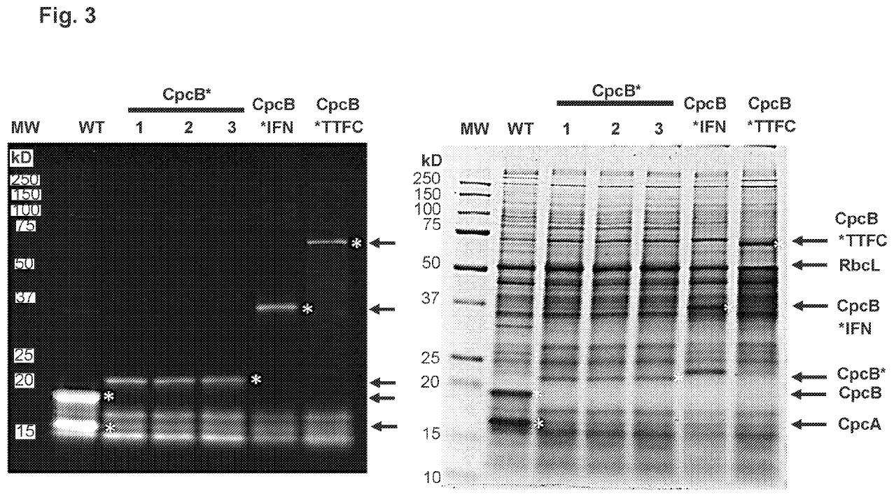

Total cell protein analysis. Analysis of total cell protein for WT and transformants was undertaken by SDS-PAGE Coomassie stain and Zn-staining for chromophore-binding polypeptide visualization (Betterle et al. 2020). The profile of the SDS-PAGE Coomassie stain (FIG. 3, WT, right panel) showed the presence of abundant CpcB (β-) and CpcA (α-) subunits of phycocyanin, migrating to about 19 and 17 kD, respectively. Also dominant is the large subunit of Rubisco (RbcL). The zinc-induced fluorescence of this SDS-PAGE protein profile, recorded by a Chemidoc imaging system (BIO-RAD) with UV irradiance as the light source, showed two brightly fluorescing bands at about 19 and 17 kD (FIG. 3, WT, left panel). These were attributed to the Zn-chromophore fluorescence of the CpcB and CpcA proteins. respectively

Three independent transformant lines with the cpcB*6xHis*tev (CpcB*) construct were similarly analyzed. These were devoid of the 19 kD CpcB protein but contained substantial amounts of a ˜21 kD band reflecting accumulation of the CpcB* protein (FIG. 3, CpcB*, right panel). This assignment was supported by the results of the Zn-chromophore fluorescence analysis, showing light emission from this ˜21 kD band (FIG. 3, CpcB*, left panel). The analysis of proteins from the CpcB*IFN strain showed substantial accumulation of a 36 kD protein attributed to CpcB*6xHis*tev*IFN, and a corresponding Zn-chromophore fluorescence band at this electrophoretic mobility position. Similarly, the analysis of proteins from the CpcB*TTFC strain showed substantial accumulation of 73 kD protein attributed to CpcB*6xHis*tev*TTFC, and a corresponding Zn-chromophore fluorescence band at this electrophoretic mobility position. It is of interest that all transformants showed traces of CpcA, as visualized from the Coomassie stain (FIG. 3, right panel) and Zn-chromophore fluorescence emission in the 17 kD electrophoretic mobility position (FIG. 3, left panel) Note that allophycocyanin proteins also showed Zn-chromophore fluorescence emissions in the 15 to 18 kD positions. However, these were distinct of the Zn-chromophore fluorescence emanating from the CpcB and CpcA proteins. These results showed presence of some CpcA in all fusion constructs and suggested the possibility of CpcA playing a role in the stable accumulation of the CpcB*, CpcB*IFN, and CpcB*TTFC proteins. This hypothesis was investigated in greater detail.

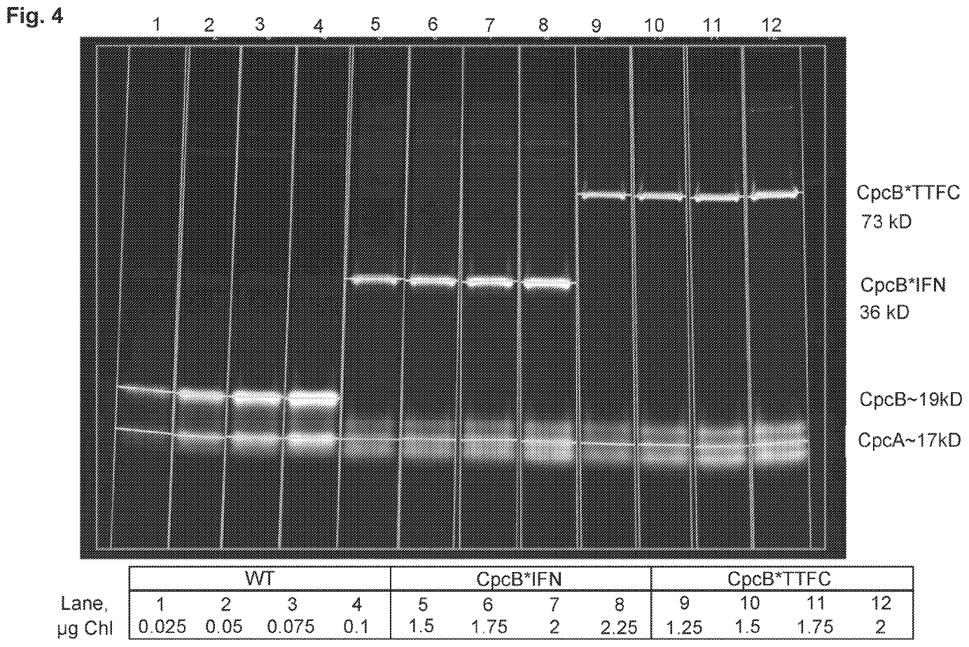

Zn-chromophore quantification in total cell extracts. To establish the ratio of CpcB and CpcA proteins in WT and transformant strains, a quantitative analysis of the Zn-chromophore fluorescence was carried out with different loadings of total cell protein extracts on multiple SDS-PAGE experiments, followed by Zn-chromophore fluorescence analysis (FIG. 4). A variable range of Chl loadings for each strain was defined to ensure an optimal signal intensity and resolution, while at the same time remaining in the linear response range for the Zn-chromophore fluorescence intensity of each band. For the WT, the range of SDS-PAGE lane loadings varied from 0.025 to 0.1 μg Chl. For the CpcB*IFN strain, a range from 1.5 to 2.25 μg Chl was optimal. For the CpcB*TTFC strain, 1.25 to 2 μg Chl loading were used. The Zn-chromophore fluorescence profile of total cell extracts from these WT, CpcB*IFN, and the CpcB*TTFC strains are shown in FIG. 4. The CpcB/CpcA Zn-chromophore fluorescence ratio for WT, CpcB*IFN and CpcB*TTFC were determined to be 1.51±0.21, 1.41±0.23, and 1.48±0.31, respectively, i.c., they proved to be statistically the same among the three samples.

The above results were interpreted to reflect the ratio of the phycocyanobilin chromophores in the CpcB and CpcA subunits. In the phycocyanin peripheral rods, there are two phycocyanobilin molecules covalently bound to the CpcB protein and one phycocyanobilin covalently bound to the CpcA (Yamanaka et al. 1978; 1982). Accordingly, a theoretical CpcB/CpcA Zn-chromophore fluorescence ratio of 2.0 was anticipated, at least for the wild type. The lower CpcB/CpcA=1.51±0.21 Zn-chromophore fluorescence ratio is probably due to a dissimilar fluorescence yield from the CpcB versus that of the CpcA subunit and should be viewed as such. Extrapolating this to the Zn-chromophore fluorescence ratio of the CpcB*IFN (=1.41±0.23), and the CpcB*TTFC (=1.48±0.31) strains leads to the conclusion that both of these transformant strains must also contain a CpcB/CpcA phycocyanobilin ratio of 2:1, or equimolar amounts of CpcB and CpcA proteins, albeit at levels lower than those seen in the WT.

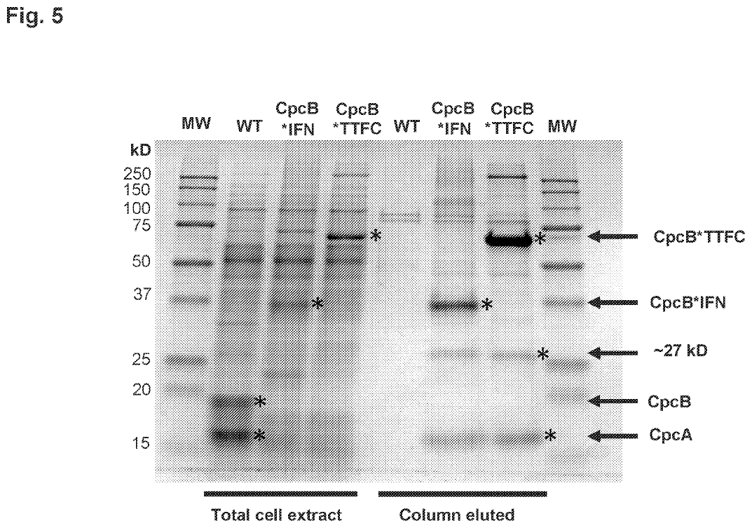

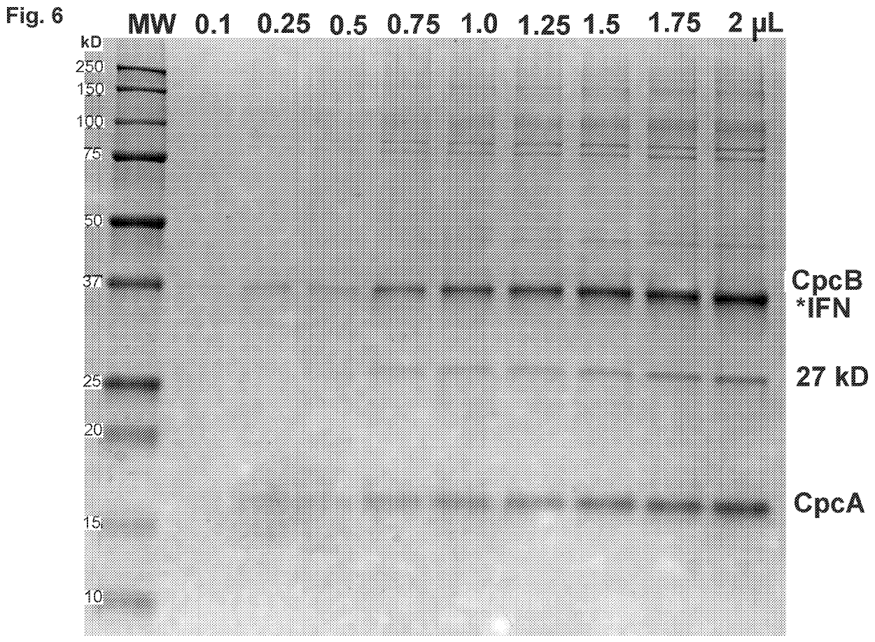

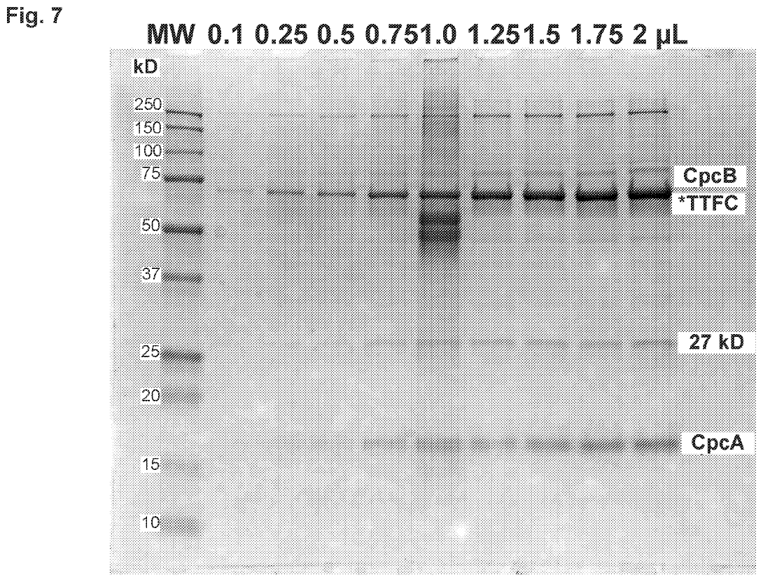

Fusion construct protein elution and analysis. The above property was investigated further using cobalt affinity chromatography and selective elution of the fusion proteins. This was performed by passing the crude cellular extracts through a His-select resin (Sigma, St. Louis, MO, United States). Such His-tag recombinant protein binding and purification enabled elucidation of the structure and composition of a fusion construct complex, unencumbered by other cellular proteins in the SDS-PAGE analysis. A side-by-side comparison of total cell extract and affinity chromatography column-eluted protein profiles is shown in FIG. 5. Marked in FIG. 5 (left panel) are the dominant CpcB and CpcA proteins for the WT, the 36 kD CpcB*IFN and the 73 kD CpcBTTFC proteins in the transformants. In the column-elution experiment (FIG. 5, right panel), no select proteins were eluted from the affinity column of the WT cell extracts, consistent with the absence of His-tagged recombinant proteins there. Column-eluted proteins from the CpcB*IFN and CpcB*TTFC crude extracts showed the anticipated 36 and 73 kD constructs. They also showed the distinct presence of a 27 kD protein and the anticipated presence of CpcA. The amount of the 27 kD protein, relative to the CpcB and CpcA was measured separately in elution experiments of proteins from the CpcB*IFN (FIG. 6) and CpcB*TTFC (FIG. 7) strains. A constant and proportional ratio of CpcB*fusion/27 kD/CpcA=3:1:3 was approximated from multiple analyses as the ones shown in FIGS. 6 and 7.

The constancy of the CpcB*fusion/27 kD/CpcA=3:1:3 ratio in either the CpcB*IFN or CpcB*TTFC fusion constructs founded the idea of a structural and possibly functional phycocyanin monomer disc in the transformant strains, which may be responsible for the successful accumulation of such heterologous proteins, when fused with the CpcB protein.

Fusion constructs comparison and presence of the phycobilisome structure. The next step comprised identification of the protein band migrating to apparent ˜27 kD that systematically eluted along with the CpcB*fusion and CpcA proteins from the His-tag affinity column. A gel slice containing the 27 kD band was excised from the SDS-PAGE and examined by mass spectrometry (see Materials and Methods). Table 1 shows the four best-matching sequencing hits, where first place with a 60.6% sequence coverage was the Phycobilisome peripheral rod-core cylinder linker polypeptide CpcG with a calculated molecular weight of 28.9 kD. This is the linker protein required for attachment of the peripheral phycocyanin rods to the core allophycocyanin cylinders in the phycobilisome of cyanobacteria (Yamanaka et al. 1978; 1982; Ughy and Ajlani 2004; Kondo et al. 2005; Watanabe and Ikeuchi 2013). Second best hit was the Photosystem I-associated linker protein CpcL with a 30.5% sequence coverage. There is a close relationship between CpcG and CpcL in each of the subgroups from different cyanobacteria, where these are encountered (Watanabe and Ikeuchi 2013), suggesting that cpcL and cpcG have the same origin but have apparently undergone independent divergence events during evolution, thereby explaining the sequence hit. C-phycocyanin β-subunit was also identified as a likely hit with 27.9% sequence coverage (Table 1). The next best was a far-removed Formyltetrahydrofolate deformylase, as an unlikely hit with 4.9% sequence coverage. These results strongly suggest that the unknown protein migrating to ˜27 kD. shown in FIGS. 5-7, is actually the 28.9 KD cpcG gene product.

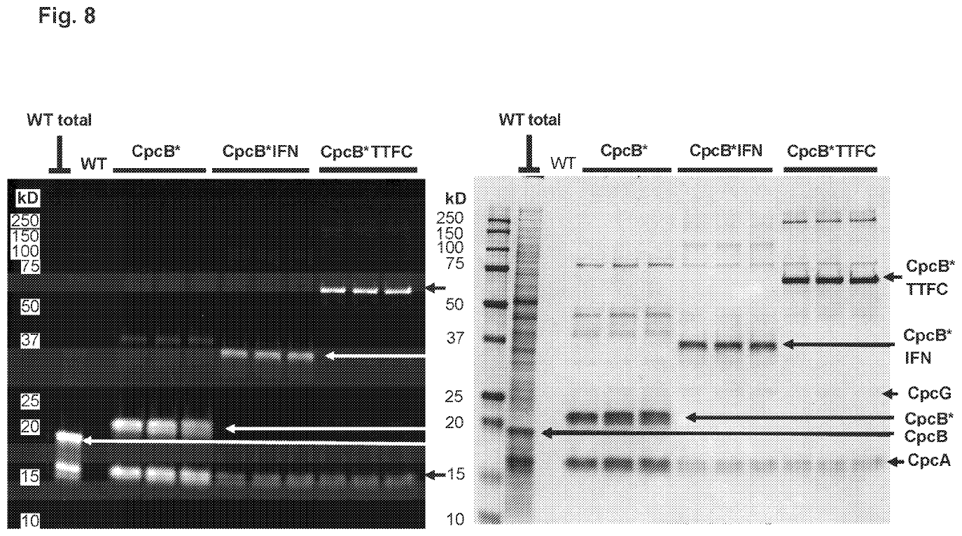

Zn-chromophore quantification in eluted fusion constructs. In an effort to test for (i.e., eliminate) the possibility that the heterologous fusion protein is the reason for the CpcB*fusion/CpcG/CpcA=3:1:3 ratio detected, a new transformant was generated (FIG. 1D), in which the 6xHis and tev DNA sequences remained fused to the cpcB gene but in the absence of a subsequent heterologous protein. This new cpcB*6xHis*tev (CpcB*) transformant was obtained by removing, through site direct mutagenesis, the coding sequence of the TTFC protein from the construct detailed in FIG. 1C. Primers used for this modification were Δttfc_fw and Δttfc_rv. (FIG. 1C; see also, Methods section below). The aim of this modification was to allow the recovery of the CpcB protein through column purification and to find out more about the elusion fraction after purification by affinity chromatography. Results from this analysis with samples in triplicate, are shown in FIG. 8. The SDS-PAGE Coomassie stain (FIG. 8, right panel) showed that no proteins were selectively eluted from the affinity chromatography of wild type cell extracts. The CpcB* mutant showed a modified CpcB protein band migrating to about 21 kD. Proteins selectively-eluted from the CpcB*IFN and CpcB*TTFC samples contained the expected 36 kD and 73 kD protein fusions, respectively. CpcA at 17 kD was present in all fusion constructs, as was the CpcG protein. FIG. 8, left panel, shows the SDS-PAGE Zn-chromophore fluorescence profile of the eluted fractions associated with the above-described proteins. These elusion profiles also showed fluorescing bands associated with the CpcB in CpcB*, CpcB*IFN, and CpcB*TTFC, as well as the CpcA. A densitometric analysis was carried out to measure the CpcB/CpcA ratio, as evidenced from the fluorescence intensity of the respective bands. These ratios were found to be: CpcB*/CpcA=1.21±0.12, CpcB*IFN/CpcA=1.30±0.04 and CpcB*TTFC/CpcA=1.40±0.13. The analysis showed no statistically significant difference between the three values, consistent with the results from the analysis of the results in FIG. 4, suggesting equimolar amounts of CpcB and CpcA in the eluted complex. It may be concluded that the CpcB*fusion/CpcG/CpcA=3:1:3 ratio in the eluted complex is independent of and forms regardless of the presence of the IFN or TTFC in the fusion constructs.

Mass spectrometry analysis of eluted proteins. Affinity chromatography eluted proteins from the transformant Synechocystis were subjected to mass spectrometric analysis to identify, by another method, protein components in the purified complexes. As a result of the total peptide sequencing analysis of these purified fractions, the same ten proteins were identified in all samples examined (Table 2). The top significant-five of these included phycocyanin components, i.e., the β-subunit of C-phycocyanin, the α-subunit of C-phycocyanin, and the phycobilisome peripheral rod-core cylinder linker polypeptide CpcG The other two entailed lower amounts of allophycocyanin α-chain and the ferredoxin-NADP reductase. More detailed information on the mass spectrometric analysis is provided in the results of Table 2.

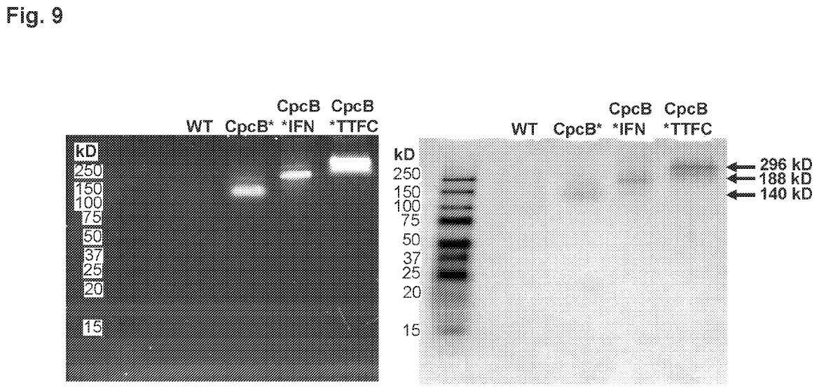

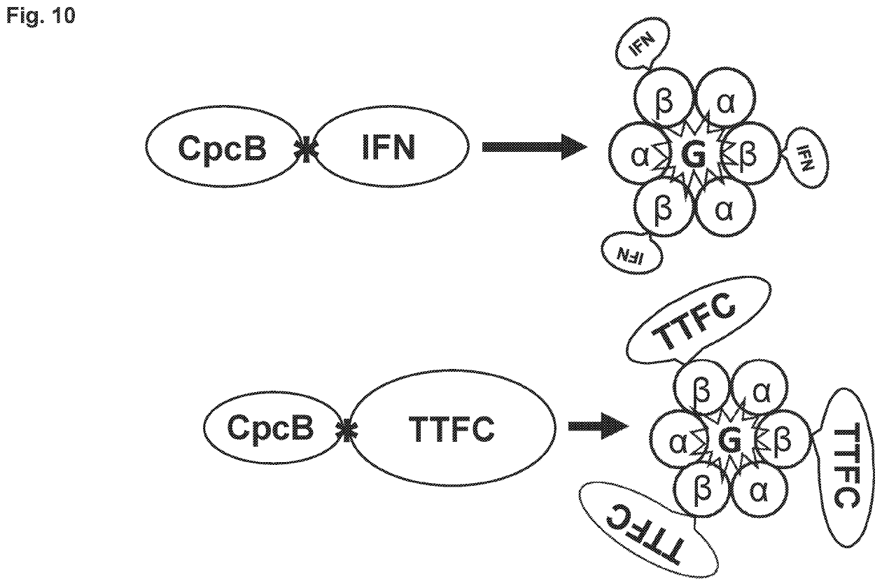

Native PAGE analysis of fusion construct eluted proteins. Selective retention during the affinity chromatography, purification, and simultaneous elution of the CpcB*fusion/CpcG/CpcA proteins in a 3:1:3 ratio suggested that they may exist and possibly function as a coherent complex. To investigate the structural association of the CpcB*fusion/CpcG/CpcA proteins, a Native-PAGE analysis of these elusions was undertaken. FIG. 9 (right panel) shows a Coomassie strain of the respective Native-PAGE analysis. FIG. 9 (left panel) shows the corresponding Zn-chromophore fluorescence analysis. It is seen that, in both analyses, no proteins were eluted from the WT, as proteins in these extracts lack the 6xHis-tag via which to bind to the affinity column. Results also showed that eluted CpcB+fusion/CpcG/CpcA proteins from the fusion constructs migrated as a single band in the Native-PAGE Coomassie and Zn-chromophore fluorescence analysis, suggesting complex formation. FIG. 9 also highlights difference in the size of the eluted complexes due to the presence of variable size heterologous fusion additions, e.g., IFN or TTFC. For the CpcB* mutant without a specific recombinant protein fused (FIG. 1D), the protein complex migrated to about 140 kD, which is consistent with the calculated 140 kD size of an (α,β)₃CpcG heterohexamer. For the CpcB*IFN construct (FIG. 1B) the protein complex migrated to the 190 kD range, which is near the calculated 188 kD size of an (α,β*IFN)₃CpcG heterobexamer. Lastly, for the CpcB*TTFC (FIG. 1C), the protein complex in FIG. 9 migrated to the 300 kD range, which is close to the 296 kD calculated size of an (α,β*TTFC)₃CpcG heterohexamer. These and the preceding results clearly showed the occurrence of a structural association in the (α,β*TTFC)₃CpcG heterohexamer complex that resembles a single phycocyanin disc, as this naturally occurs in cyanobacteria, with the proximal to the core cylinders CpcG linker polypeptide.

A schematic of the minimal stable such complex is shown in FIG. 10, in which a heterohexameric disc is composed of 3CpcA α- and 3CpcB β-phycocyanin subunits. The recombinant fused proteins, depicted as IFN and TTFC in this schematic, emanate radially from the heterohexameric disc, whereas the linker CpcG protein occupies the disc center. Not to be bound by theory, this structure is apparently recognized by the cells as a native feature, explaining how the cell tolerates the presence and enables its substantial accumulation.

Functional analysis of the heterohexameric (α,β*Fusion)₃CpcG complexes. Possible association of the (α,β*Fusion)₃CpcG heterohexameric complex and function as a residual phycocyanin antenna size in the transformants was investigated by chlorophyll fluorescence and sensitive absorbance spectrophotometry measurements of the effective light-harvesting antenna size of photosystem-II. The absorbance spectra of cell suspensions were measured to evaluate the pigment profile in WT, CpcB*, CpcB*IFN, CpcB*TTFC and Δcpc strains (FIG. 11). For this set of measurements, cell suspensions in buffer were lysed by French press treatment to minimize scattering, and the absorbance spectra were measured from 750 to 550 nm (FIG. 11). The absorbance spectra were normalized to the Chl max at 678 nm so that differences of the absorbance at 620 nm could be seen among the various transformants. WT cells showed the typical absorbance bands of chlorophyll peaking at 678 nm and phycocyanin at 620 nm. The Δcpc strain (FIG. 1E) lacked the entire cpc operon genes and, therefore, lacked the CpcB and CpcA phycocyanin proteins due to the cpc operon deletion (Kirst et al. 2014). The absorbance spectrum of the Δcpc strain showed a low amplitude peak and 620 nm, attributed to a Chl secondary absorbance in the red spectral region. The absorbance spectra of CpcB*Fusion constructs, e.g., CpcB*, CpcB*IFN and CpcB*TTFC cells, showed a slightly elevated absorbance at 620 nm (FIG. 11 and Batterle et al. 2019), consistent with the presence of some phycocyanobilin chromophores in these strains. The amplitude of this absorbance, however, was considerably lower than that of the WT.

Possible functional association of the (α,β* Fusion)₃CpcG heterohexameric complex as a residual phycocyanin antenna size in the transformants was investigated with intact and DCMU-inhibited Synechocystis cells from (i) the yield Φ of Chl a fluorescence and (ii) the functional PSII absorption cross section to 620 nm light. For these measurements, strains were suspended in the 1.5 mm pathlength spectrophotometer cuvette in the range of 32 to 42 μg Chl mL⁻¹. Then, weak actinic excitation at 10 μmol photons m⁻² s⁻¹ was provided at 619.5 nm by a narrow-bandpass Baird Atomic interference filter coupled with a 659.6 nm visible bandpass negative cut-off Ealing filter. Table 3 [Chl] shows the Chl loading in the spectrophotometer cuvette. The raw Chl a fluorescence yield data in μV signal from the apparatus and, in parenthesis, the Chl a fluorescence yield of the various strains normalized to the same [Chl] content and reported relative to that of the Δcpc are also shown. It is evident that all fusion transformants exhibited a greater yield of Chl a fluorescence and roughly in proportion to their elevated absorbance at 620 nm. More specifically, Φ(IFN) was 1.84× greater than Φ(Δcpc), whereas Φ(TTFC) and Φ(CpcB*) were 1.66× and 1.74× greater than Φ(Δcpc). These results indicate that, under these experimental conditions, higher rates of excitation energy arrived at PSII in the fusion transformants, presumably because more actinic light was harvested (greater antenna size) in the latter than in the Δcpc strain.

Direct rates of PSII absorption cross section were measured from the rate constant k_{II} of PSII photochemistry, measured from the fluorescence induction kinetics of intact cells suspended in the presence of either 12 or 24 μM DCMU (Table 3). Under weak actinic excitation, rates of PSII photochemistry are directly proportional to the light-harvesting antenna size, which depends on the number of pigment molecules acting as antennae for this photosystem. This has previously been shown to be a direct method for the measurement of the photosystem effective antenna size (Melis 1989). DCMU concentrations at 12 and 24 μM were used in these measurements with similar results, k_{II}-1 and k_{II}-2 for samples in the presence of 24 μM DCMU (Table 3) show the rate constants (rates of light absorption) obtained upon a first illumination of dark-adapted cells (k_{II}-1 ), followed by a 2-min dark relaxation of the redox state of PSII and upon a second illumination and fluorescence kinetic registration of the same sample (k_{II}-2). The repeat measurement was undertaken to test for sample stability and signal reproducibility in subsequent illuminations in the presence of DCMU. Consistent with the fluorescence yield Φ measurement, k_{II}-IFN was on the average 1.46× greater than k_{II}-Δcpc, whereas k_{II}-TTFC and k_{II}-CpcB* were 1.39× and 1.57× greater than k_{II}-Δcpc. These results are strong evidence that the (α,β*IFN)₃ CpcG, (α,β*TTFC)₃ CpcG, and (α,β*)₃ CpcG heterohexameric complexes exist in a functional association with the core allophycocyanin cylinders in the fusion transformants and that they functionally transfer excitation energy from the CpcB*Fusion and CpcA chromophores to the PSII reaction center thereby contributing to PSII photochemistry.

### Analysis of Technical Results

Over-expression of heterologous proteins as fusion constructs in cyanobacteria, with the CpcB β-subunit of phycocyanin as the leader sequence, have been documented in the literature. Examples include divergent proteins, ranging from the isoprene synthase from kudzu (Chaves et al. 2017), the β-phellandrene synthase from a variety of plant sources (Formighieri and Melis 2017; 2018; Betterle et al. 2018), the geranyl diphosphate synthase from grand fir (Betterle et al. 2019), the geranyl linalool synthase from tobacco (Formighieri and Melis 2017), as well as the human interferon α-2 protein (IFN) (Betterle et al. 2020), and the bacterial tetanus toxin fragment C (TTFC) (Zhang et al. 2021). See also, WO20201050968, WO2017205788, and WO2016210154. A working hypothesis for such over-expressions, amounting up to 20% of the total cell protein, could be based on the assumption that CpcB*Fusion proteins accumulated as soluble and stable proteins in the cytosol of the cyanobacteria, retaining the activity of the heterologous trailing moiety but preventing the assembly of peripheral phycocyanin rods (Chaves et al. 2017; Formighieri and Melis 2917; Betterle and Melis 2019).

The results presented in the present illustrative embodiments provide a strikingly different expression model, compared to expression of fusion proteins as soluble proteins, comprising the following properties:
(i) The CpcB*Fusion proteins assemble as functional (α,β*P)₃CpcG heterohexameric discs, where α is the CpcA phycocyanin subunit protein, β*P is CpcB*Fusion protein, and CpcG is the 28.9 kD phycocyanin linker polypeptide.
(ii) The CpcA α-subunits and CpcB β-subunits in the (α,β*P)₃CpcG complex covalently bind the physiological number of open tetrapyrrole bilin chromophores, i.e., one bilin per α-subunit and two bilins per β-subunit.
(iii) The (α,β+P)₃CpcG heterohexameric disc is functionally attached to the Synechocystis AP core cylinders and efficiently transfers excitation energy from the assembled (α,β*P)₃CpcG heterohexameric phycocyanin subunits to the PSII reaction center for charge separation and photochemical electron transfer.
(iv) In addition to the IFN and TTFC tested in this work, protein P in the (α,β*P)₃CpcG heterohexameric disc could be the isoprene synthase (Chaves et al. 2017), the β-phellandrene synthase (Formighieri and Melis 2018), the geranyl diphosphate synthase (Betterle et al. 2019), or the geranyl linalool synthase (Formighieri and Melis 2017) all of which retained their original catalytic activity in the respective fusion constructs. These observations suggested that the heterologous fusion protein P is distal to the (α,β*P)₃ compact disc (FIG. 10), i.e., it is exposed to the aqueous cytosolic medium so that it does not interfere with the anchoring of the (α,β*P)₃ heterohexamer onto the Synechocystis AP core cylinders (FIG. 12). Accordingly, the (α,β*P)₃CpcG heterohexameric disc (David et al. 2011) performs a dual function, comprising sunlight absorption and excitation energy transfer from the α,β phycocyanin to AP and the PSII reaction center, while the fused heterologous protein P performs its native physiological enzymatic catalysis under in vivo conditions in the Synechocystis cytosol.

The main evidence for the existence of a structural and functional complex associated with the heterologous protein fused to the CpcB subunit includes the CpcB/CpcA subunit ratios, which were similar in transformant and the WT strains (FIG. 4). The next piece of evidence in support of the proposed configuration is the elusion profile of the purified fractions of strains CpcB*IFN and CpcB*TTFC, where the fusion protein, the CpcA subunit and the 28.9 kD CpcG linker protein all eluted together (FIG. 5-7), in spite of the fact that only the CpcB*Fusion was endowed with the 6xHis tag for selective column affinity chromatography. The necessity of a functional association of the (α,β*P)₃CpcG complex with the AP core cylinders receives further support from the observation that PC discs cannot accumulate in the cyanobacterial cell unless they are functionally associated with the AP core cylinders or the thylakoid membrane through the CpcG linkers (Kondo et al 2005; 2007; 2009). Accordingly, key to the stability of the many CpcB*Fusion proteins examined so far is that the latter, as part of the (α,β*P)₃CpcG heterohexameric complex, comprise a functional PC disc. Otherwise, a free floating CpcB*Fusion protein would not be stable and would be degraded due to the presence and activity of Clp proteases in the cell (Baier et al 2014).

The organization, functionality, and spatial arrangement of the different elements of the PBS in cyanobacteria is ensured by linker polypeptides, which provide the necessary structural support and proximity to make light capture and efficient excitation energy transfer to the photochemical reaction center (Watanabe and Ikeuchi 2013, Chang et al. 2015). These linkers can be grouped according to their role and location in the PBS superstructure. Included are the AP cylinder-thylakoid membrane linkers, which are involved in the core cylinder interaction with the chlorophyll-proteins of PSII, followed by the core cylinder assembly linkers, the proximal PC rod-AP core cylinder linkers, which mediate the association between peripheral rods and the core cylinders and, lastly, the distal rod linkers, involved in rod disc assembly and extension (Sidler 1994; Ughy and Ajlani 2004; Liu et al. 2005; Guan et al. 2007). The proximal PC rod-AP core cylinder linkers (CpcG) are important in the context of this work, as they are primarily responsible for the structural and functional association of the peripheral PC rods, and of the (α,β*P)₃CpcG complex, to the AP core cylinders. In Synechocystis, two homologues of the cpcG gene exist, i.e., the cpcG1 and cpcG2, and have been described in the literature (Kondo et al. 2005; 2007; 2009). The consistent presence of the 28.9 kD cpcG gene product in the transformants examined in this work offers evidence that the (α,β*P)₃heterohexamer is the phycocyanin disc proximal to the AP core cylinders. Conversely, absence of the 33 kD CpcC1 and 30 kD CpcC2 linkers is consistent with the absence of the middle and distal PC discs in the CpcB*IFN and CpcB*TTFC transformants.

Fusion constructs of heterologous proteins with the CpcB subunit of PC (FIG. 1) comprised a successful approach in Synechocystis for the overexpression, up to 20% of the total cell protein, of the human interferon α-2 protein (IFN) and the tetanus toxin fragment C (TTFC). The strains containing IFN and TTFC (FIGS. 1B and 1C) can be used for the commercial production of these biopharmaceutical proteins. The transformant cpcB*L*H*tev containing the 6xHis tag and tev cleavage site encoding DNA (FIG. 1D) served as an additional control to simulate the cpc genomic DNA structure of the wild type, with the 6xHis tag present to facilitate the selective affinity chromatography purification process and mass spectrometry analysis of the CpcB protein and associate polypeptides. This transformant also failed to assemble more than the proximal (α,β*)₃CpcG heterohexameric disc, in spite of the absence of a fusion protein P, suggesting that minor modifications to the C-terminus of the CpcB subunit could bring about changes in the spatial arrangement of the PC discs so as a prevent the full elongation of the PC peripheral rods.

The mass spectrometric analysis of the selective affinity-purified complex consistently showed presence of the (α,β*P)₃CpcG heterohexameric disc components. Qualitatively, it also showed the occasional presence of other possibly related cyanobacterial compounds (Table 2). Among those, the Ferredoxin-NADP⁺ reductase (FNR) was present in the eluted fractions. FNR catalyzes the electron transfer reaction between reduced ferredoxin and NADP⁺, and is reportedly localized at the proximal or distal PC disc of the PBS (Arteni et al 2009; Gomez-Lojero et al 2003; Van Thor et al 1999). Such FNR adherence to PC may explain the elution of FNR along with the (α,β*P)₃CpcG heterohexameric disc from the affinity chromatography column. Additional PC linker polypeptides were occasionally detected in some of the transformants, e.g., the CpcC1 and CpcD, but these were not consistently present in the elution fractions. However, the occasional occurrence of these linkers in the affinity purified fractions indicated that the formation of the middle PC disc, adjacent to the proximal to the AP core disc, may have had a tendency to also assemble in the transformants, albeit not quantitatively or reproducibly, and certainly not with all the transgenes examined due to steric hindrances generated by the presence of the heterologous protein.

In summary, evidence provided in this section showed that the CpcB*Fusion proteins, multiple examples of which have been shown to stably accumulate in cyanobacterial transformants, comprise an (α,β*P)₃CpcG heterohexameric fusion protein complex (FIG. 10). which assembles in a functional association with the AP core cylinders, absorbing sunlight and transferring excitation energy from the α,β PC subunits to the PSII reaction center (FIG. 12). The peripherally-oriented heterologous protein P is exposed to the soluble cytosol and retains catalytic activity, when P is an enzyme rather than a structural protein.

### 2. cpcA*IFN and cpcG1*IFN Constructs Provide Target Protein Expression at a Level of 6.4% of Total Cellular Protein

### Design of Fusion Construct cpcA*IFN

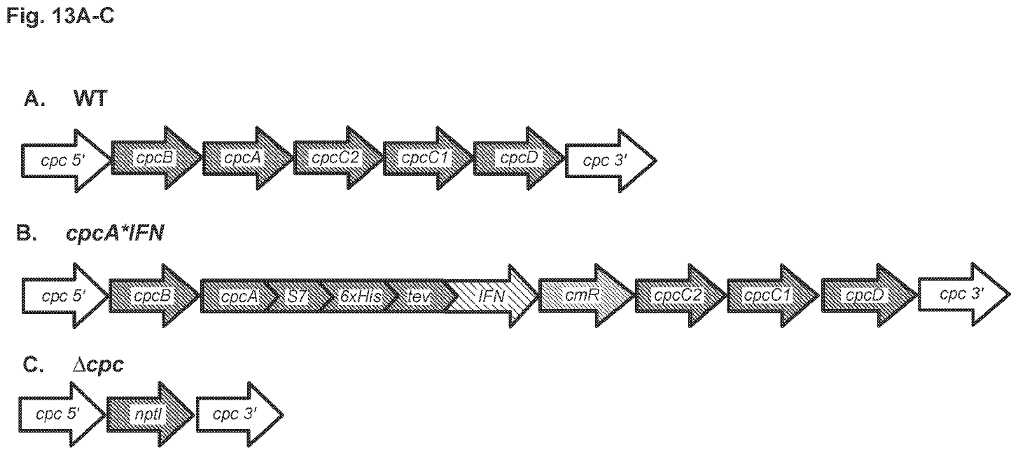

Relative to the wild type cpc operon in Synechocystis (FIG. 13A), a DNA construct was designed, comprising a fusion between cpcA, encoding the α-subunit of phycocyanin, and the human interferon α-2 (IFN) genes (FIG. 13B). Sequence information for this and the other constructs used in this technical section is provided at the end of this section. The cpcA*IFN DNA construct was designed to replace the native cpcA gene in the cpc operon, and also included spacers (S7, 6xHis, tev) and the cmR gene, conferring chloramphenicol antibiotic resistance to these transformants. As noted above, the cpcA gene encodes the ˜17 kDa α-subunit of phycocyanin, which, in the wild type, complexes with the ˜19 kDa β-subunit to make the basic (α,β)₃ heterohexameric phycocyanin disc unit in cyanobacteria (Ughy and Ajlani 2004; Kondo et al. 2005; Kirst et al. 2014).

The cpcA and IFN sequences in the cpcA*IFN fusion construct (FIG. 13B) were separated with a segment of DNA encoding a seven amino acid spacer (S7). The construct also included DNA sequences encoding a His tag (6xHis), and a Tobacco Etch Virus Protease cleavage site (tev), the latter of which facilitates in vitro enzymatic cleaving of the leader and trailing proteins (Zhang et al. 2021). These spacer additions did not interfere with the over-expression of phycocyanin subunit fusion constructs (Betterle et al 2020). The nucleotide and amino acid sequences of the CpcA*S7*6xHis*tev*IFN*cmR construct are provided at the end of this section.

Following transformation and antibiotic selection, attainment of transgenic DNA copy homoplasmy in the transformant strains was confirmed through genomic DNA PCR analysis (not shown).

### Protein Analysis of Total Cell Extracts from the cpcA*IFN Fusion Transformants

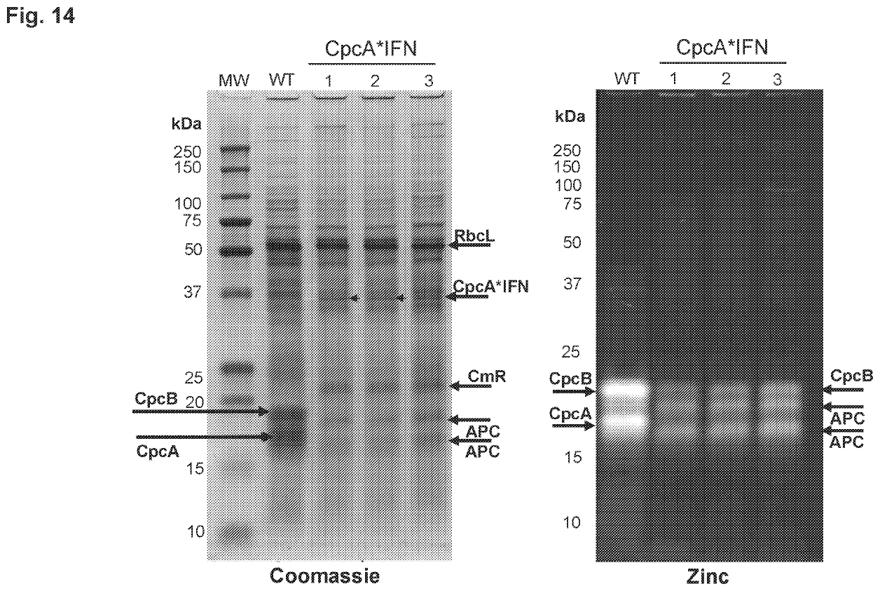

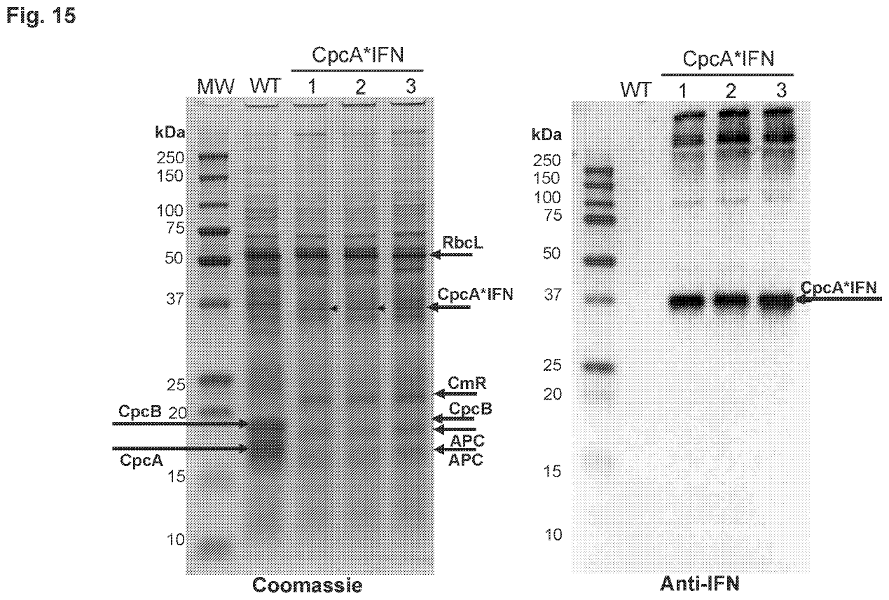

A combined approach to protein analysis from WT and cpcA*IFN fusion transformants of Synechocystis was implemented through SDS-PAGE followed by Coomassie blue staining, Zinc-chromophore fluorescence (FIG. 14), and Western blot analysis (FIG. 15). Three biological replicates from independent transformant lines were used to compare the cellular protein profile against that of the WT.

FIG. 14 (left panel) shows the dominant presence of the CpcB β-subunit and CpcA α-subunit of phycocyanin in the WT transoformants, migrating to ˜19 and ˜17 kDa, respectively. The CpcA*IFN transformant strains lacked the individual ˜17 kDa CpcA proteins and showed, instead, a new protein band migrating to ˜34 kDa, attributed to the CpcA*IFN fusion protein. FIG. 14, left panel, also shows the electrophoretic mobility of RbcL, the large subunit of Rubisco, migrating to about 56 kDa, which was present in all samples. Quantitative gel scanning measurements showed that the ˜34 kDa CpcA*IFN fusion protein accounted for 3.04%±0.01 of the total cellular protein, as measured from the Coomassie blue staining of the bands.

To visualize the possible association of phycocyanobilin pigments with the protein bands in FIG. 14, left panel, SDS-PAGE-resolved proteins were subjected to Zinc-staining (Betterle et al. 2020). In wild type (FIG. 14, right panel) Zn-chromophore fluorescence emanated from the CpcB, CpcA and allophycocyanin (APC) proteins. In the CpcA*IFN transformants, Zn-chromophore fluorescence was observed from residual 19 kDa CpcB and from allophycocyanin (APC) proteins, but not from the 17 kDa CpcA electrophoretic mobility position. A low-yield Zn-chromophore fluorescence could be discerned emanating from the ˜34 kDa position, attributed to pigments in association with the CpcA in the CpcA*IFN fusion protein. In this case, the low yield of the CpcA*IFN fusion protein is probably due to the position and influence of the IFN on the Zn-chromophores of the CpcA. Results from the Zn-chromophore fluorescence analysis is consistent with the assignment of protein bands to CpcA*IFN, CpcB and CpcA in the wild type and transformants.

Western blot analysis with specific polyclonal antibodies raised against the human IFN protein was used to further test the identity of the various protein bands shown in FIG. 14. FIG. 15, right panel, shows a positive cross-reaction between anti-IFN polyclonal antibodies and the CpcA*IFN fusion protein at ˜34 kDa in all transformant lines tested. Moreover, two distinct cross-reactions of substantial intensity were also detected with protein bands at a much higher MW range (>250 kDa), specifically in the CpcA*IFN samples, suggesting presence of higher order complexes containing the CpcA*IFN fusion protein. The higher MW bands (>250 kDa) likely originated from undissociated (α*IFN,β)₃CpcG1 oligomeric phycocyanin discs (please see below). Quantification of the higher MW bands (>250 kDa) suggested an extra 3.36%±0.07 CpcA*IFN content to the total cell protein. resulting in an overall CpcA*IFN content of 6.4%±0.08 of the total cellular protein. Resolved proteins from wild type cells did not show any cross-reactivity with the anti-IFN antibodies, supporting the notion of high specificity of the anti-IFN immune sera.

### Absorbance Spectral Analysis of the (α*IFN,β)₃CpcG1 Mutants

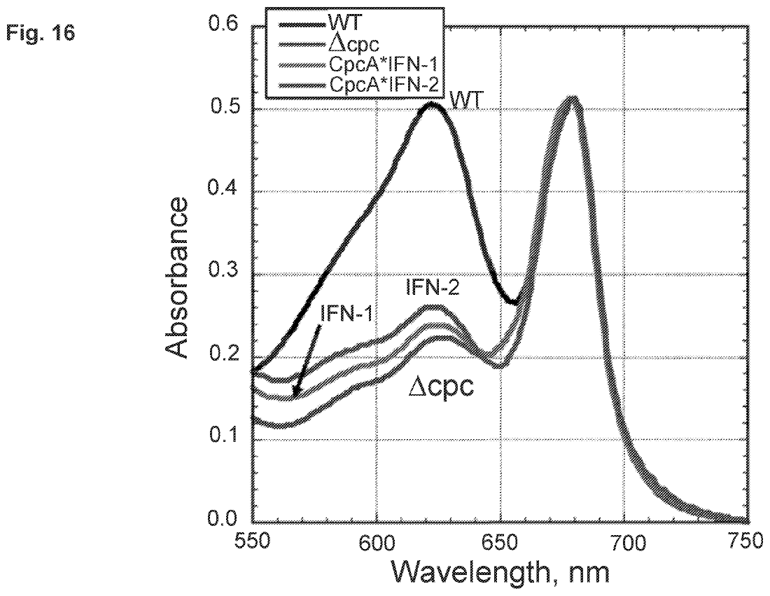

Absorbance spectra of cell suspensions were measured to evaluate the pigment profile of the (α*IFN,β)₃CpcG1 mutant relative to that of the wild type and Δcpc strains (FIG. 16). The absorbance spectra were normalized to the chlorophyll max at 678 nm so that differences of the absorbance at 620 nm, where phycocyanin absorbs, could be more easily assessed among the various strains. WT cells showed the typical absorbance bands of chlorophyll a peaking at 678 nm and phycocyanin at 620 nm. The Δcpc strain (FIG. 13C) lacked the entire cpc operon genes and, therefore, lacked the CpcB and CpcA phycocyanin proteins due to the cpc operon deletion. The absorbance spectrum of the Δcpc strain showed a lower amplitude peak at 620 nm, which is attributed to a chlorophyll a secondary absorbance in this spectral region.

The absorbance spectra of the (α*IFN,β)₃CpcG1 constructs (FIG. 13B) showed a somewhat-variable low-level enhancement in the 620 nm region (FIG. 16), suggesting the presence of measurable amounts of phycocyanobilin chromophore and phycocyanin pigment in these strains. The average ratio of phycocyanin absorbance in the (α*IFN,β)₃CpcG1 transformants versus that in the wild type from three independent biological replicates, measured as A(620 nm,CpcA*IFN)/A (620 nm,WT), was equal to 8.25%±3, i.c., indicating presence of roughly one twelfth of the wild type phycocyanin in this mutant.

### Functional Antenna Analysis of the (α*IFN,β)₃CpcG1 Complex

The functional association of the (α*IFN,β)₃CpcG1 beterohexameric complexes as a residual phycocyanin antenna in the transformants was investigated by sensitive absorbance spectrophotometry, measuring the functional PSII absorption cross section to 620 nm light, and from the yield Φ of chlorophyll a fluorescence of intact and DCMU-inhibited Synechocystis. The rate constant k_{II} of PSII photochemistry was measured from the fluorescence induction kinetics of intact cells suspended in the presence of 12 μM DCMU (Table CpcA*IFN) (Melis and Duysens 1978; Melis 1989). Under weak actinic excitation, rates of PSII photochemistry are directly proportional to the number of pigment molecules acting as antennae for this photosystem. Under these conditions, k_{II} values show the rate constants (rates of light absorption) obtained upon illumination of dark-adapted cells. It is evident from these results that k_{II}-(CpcA*IFN=7.33 s⁻¹) are slightly greater than k_{π}-(Δcpc=6.60 s⁻¹), and substantially lower than that of the k_{II}-(WT=24.4 s⁻¹) (Table CpcA*IFN, k_{II}-s⁻¹ column).

A measure of the direct phycocyanin contribution to the rate of photochemistry was obtained upon subtracting the contribution of chlorophyll a in the Δcpc k_{II} from the k_{II} value of the (α*IFN,β)₃CpcG1 transformants and from that of the wild type. This is shown in Table 4. column k_{II}s⁻¹ (minus the k_{II} of Δcpc). This analysis showed that phycocyanin in the (α*IFN,β)₃CpcG1 mutant transferred excitation energy to PSII at about 4.1% rate, when compared to phycocyanin in the wild type (average k_{II} of 0.73 s⁻¹ versus 17.8 s⁻¹, respectively). This 4.1% value is somewhat lower from the fraction of phycocyanin present in the (α*IFN,β)₃CpcG1 mutant (8.25%) relative to that in the wild type, and also lower from the calculated amount of the CpcA*IFN protein in the mutant (6.4%), suggesting that excitation energy transfer does not occur efficiently from the (α*IFN,β)₃CpcG1 complexes to the PSII reaction center.

The chlorophyll a fluorescence yield Φ of the various strains examined in this section of the detailed technical section is also shown in Table 4 (Fluorescence yield). The yield data normalized to that of chlorophyll loaded in the cuvette and, in parentheses, further normalized to that of the Δcpc strain (=1.0) are shown in Table 4 (Fluorescence yield normalized to Chl loaded). On average, Φ((α*IFN,β)₃CpcG1) was about 1.59-fold greater than Φ(Δcpc). By comparison, the wild type Φ(WT) was about 3.41-fold greater than Φ(Δcpc). These Φ results are also consistent with the notion of a rather limited contribution of pigments in the (α*IFN,β)₃CpcG1 complex to PSII photochemistry.

It is noted that extrapolation of fluorescence measurements to estimates of PC content in the mutant and WT strains is not as robust compared to pigment absorbance and k_{II} data, as the fluorescence method is indirect and there could be yield differences among the three types of strains (Δcpc, (α*IFN,β)₃CpcG1, and WT) employed in this measurement. Accordingly qualitative assessment was employed, rather than quantification.

### Design of Fusion Constructs cpcG1*IFN and Double Fusion (cpcB*TTFC+cpcG1*IFN)

The following describe heterologous expression of proteins fused independently to the CpcB phycocyanin β-subunit and the CpcG1 phycocyanin-allophycocyanin rod-core cylinder linker. Further, a double fusion (cpcB*TTFC+cpcG1*IFN) of the two genes was examined in the model cyanobacteria Synechocystis sp. PCC 6803 (Synechocystis).

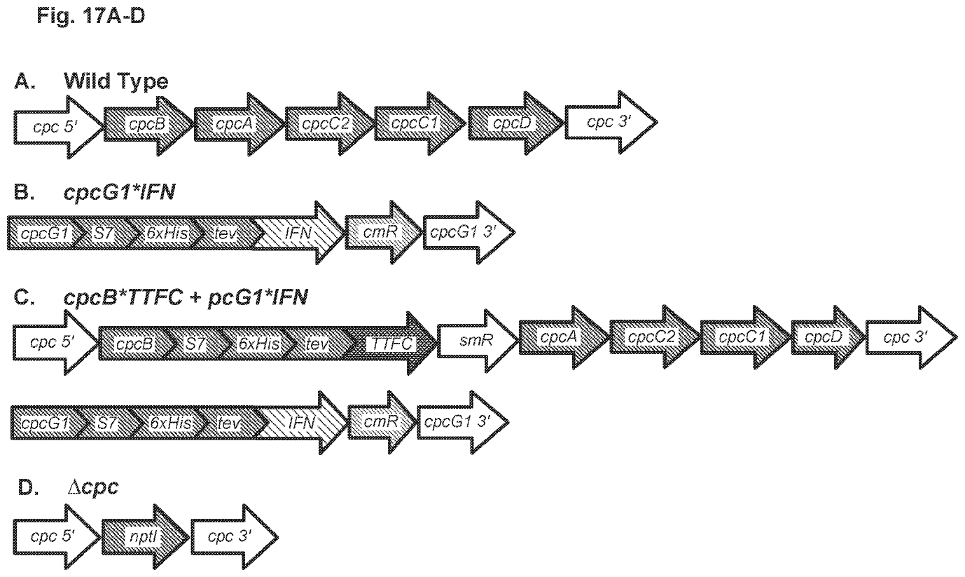

Wild type and the transformants cpcB*S7*6xHis*tev*TTFC (abbreviated as cpcB*TTFC) were used as recipient strains to obtain cpcG1*S7*6xHis*tev*IFN (abbreviated as cpcG1*IFN) and double fusion strain cpcB*TTFC+cpcG1*IFN. The presence of the S7providing distancing between the CpcG1 and IFN proteins, conferring a tertiary configuration allowing the TEV enzyme to access the tev cleavage site and, thus facilitate cleaving of the two proteins, thereby releasing a native form of the target (IFN) enzyme (Zhang et al. 2021). FIG. 17 shows in greater detail the map of the different constructs used in this technical description to transform the genomic DNA of wild type (WT) Synechocystis (FIG. 17A). A DNA construct comprising a fusion between the cpcG1 and IFN genes (FIG. 17B, cpcG1*IFN) is shown. Sequence information for this and the other constructs used in these illustrative embodiments is supplied in the Illustrative Expression Constructs and Sequences section below. The cpcG1*IFN DNA construct was designed to replace the native cpcG1 gene, and also included the cmR gene, conferring chloramphenicol antibiotic resistance to these transformants. The cpcG1 gene encodes a linker protein that binds the proximal (α,β)₃(α,β)₃ heterohexameric phycocyanin dimer disc to the core allophycocyanin complexes in the cyanobacterial phycobilisome (Ughy and Ajlani 2004; Kondo et al. 2005). Construct cpcB*TTFC (FIG. 17C) was designed to replace the cpcB gene in the cpc operon with the fusion construct cpcB*TTFC, followed by the smR (spectinomycin) resistance cassette. The strain harboring the cpcB*TTFC fusion construct was additionally transformed to install the cpcG1*IFN construct in the cpcG1 locus. FIG. 17D shows the Δcpc construct, in which the entire cpc operon was deleted upon replacement with the kanamycin resistance (nptI) cassette. These cells lacked phycocyanin due to a cpc operon deletion and contained only the allophycocyanin core cylinders (Kirst et al. 2014).

The cpcB and TTFC DNA sequences in the cpcB*TTFC construct, as well as the cpcG1 and IFN sequences in the cpcG1*IFN fusion construct were spaced with a piece of DNA encoding a seven amino acid spacer (S7) in order to distance the two proteins, the His tag (6xHis) to enable a differential column affinity chromatography elution of the fusion construct proteins, and the Tobacco Etch Virus Protease cleavage site (tev) to facilitate in vitro enzymatic cleaving of the leader and trailing proteins (Zhang et al. 2021). Nucleotide sequences and spacers employed for these constructs are provided the Illustrative Expression Constructs and Sequences section below.

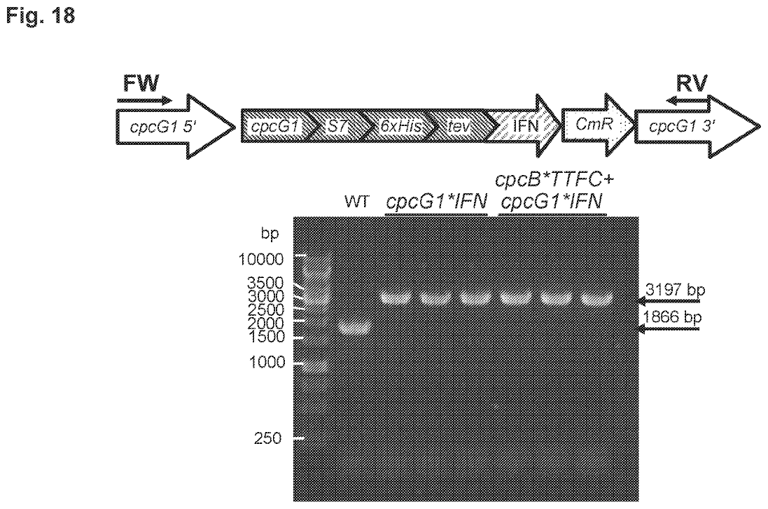

Following transformation and antibiotic selection, attainment of transgenic DNA copy homoplasmy in the transformant strains was tested through genomic DNA PCR analysis (FIG. 18). Primers cpcG1-5′ and cpcG1-3′ were designed from the flanking regions of the transgenic DNA insertion site. PCR amplification using WT genomic DNA as a template generated a single product of 1,866 bp, while DNA from the transformant cpcG1*IFN strains generated a single product size of 3,197 bp. Absence of wild type gene products in either the cpcG1*IFN or double transformant cpcB*TTFC+cpcG1*IFN strains suggested that they have reached a state of homoplasmy with respect to the mutant DNA.

### Protein Analysis of Total Cell Extracts from CpcG1*IFN and Double Fusion CpcB*TTFC+CpcG1*IFN Transformants

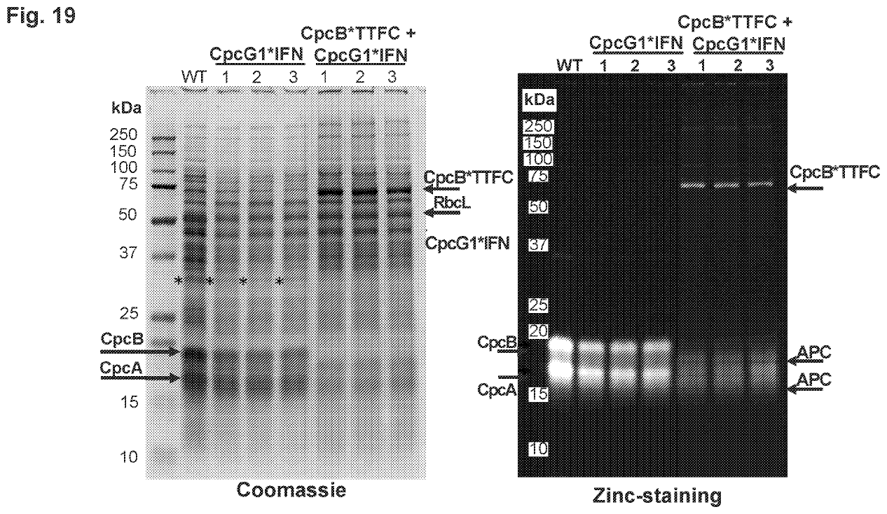

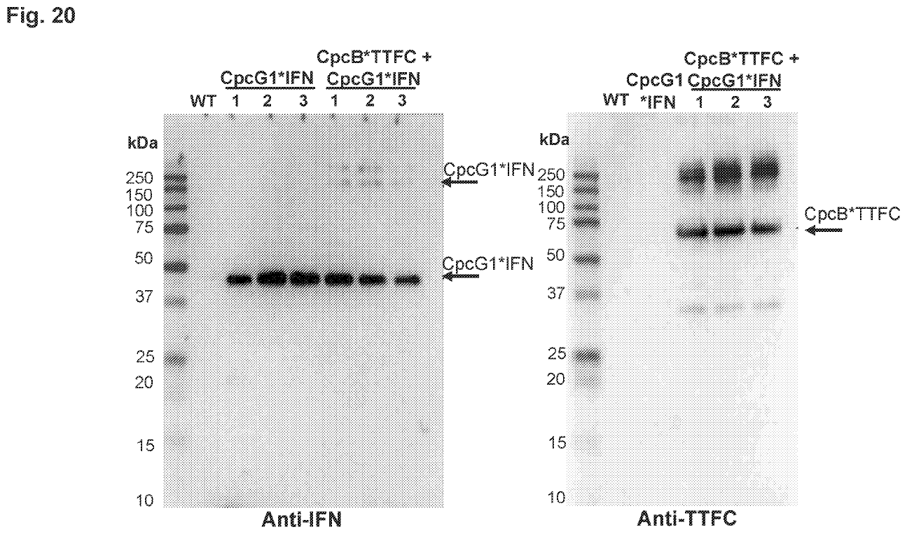

A first approach to protein analysis from WT and transformant Synechocystis was implemented through SDS-PAGE followed by Coomassie blue staining, Zinc chromophore fluorescence (FIG. 19), and Western blot analysis (FIG. 20). Three biological replicates from independent transformant lines were used to compare the cellular protein profile against that of the WT. FIG. 19 (left panel) shows the dominant presence of the CpcB β-subunit and CpcA α-subunit of phycocyanin in the WT, migrating to ˜19 and ˜17 kDa, respectively. Somewhat lower amounts of the ˜19 kDa CpcB and ˜17 kDa CpcA are also present in the CpcG1*IFN strains. The CpcB*TTFC+CpcG1*IFN double transformant strains lacked the individual ˜19 and ˜17 kDa proteins and instead shows a dominant protein band migrating ˜73 kDa, attributed to the CpcB*TTFC fusion protein. Quantitative gel scanning measurements showed that the 73 kDa CpcB*TTFC fusion protein accounted for about 10.7% (±0.9) of the total cellular protein, as measured from the Coomassie blue binding to proteins. FIG. 19, left panel, also shows the electrophoretic mobility of RbcL, the large subunit of Rubisco. migrating to about 56 kDa, which was present in all samples.

The CpcG1*IFN fusion protein band could be measured only in relation to the amount of RbcL in each lane, due to the presence of proteins in Synechocystis with similar molecular weight that migrate in the ˜46 kDa region. Quantitative gel scanning measurements, and by using the RbcL-to-46 kDa Coomassie stain ratio as a reference. showed that the ˜46 kDa CpcG1*IFN fusion protein specifically accounted for about 3% (±0.67) of the total cellular protein, both in the CpcG1*IFN and CpcB*TTFC+CpcG1*IFN transformants.

To visualize the possible association of phycocyanobilin pigments with the protein bands shown in FIG. 19, left panel, SDS-PAGE-resolved proteins were subjected to Zinc-staining (Betterle et al. 2020). Results (FIG. 19, right panel) showed Zn-chromophore fluorescence emanating from the CpcB, CpcA and allophycocyanin (APC) proteins in the wild type and CpcG1*IFN transformants. In the CpcB*TTFC+CpcG1*IFN double transformant strains, Zn-chromophore fluorescence was seen from the CpcA and allophycocyanin (APC) proteins but not from the 19 kDa CpcB electrophoretic mobility position. Instead, there was a pronounced Zn-chromophore fluorescence emanating from the 73 kDa position, attributed to bilin pigments in association with the CpcB in the CpcB*TTFC fusion protein. Zn-chromophore fluorescence was not detected in the electrophoretic mobility position of the CpcG1*IFN fusion protein (˜46 kDa), as this complex does not contain phycocyanobilin pigments. The Zn-chromophore fluorescence analysis is consistent with the assignment of protein bands to CpcB, CpcA, and CpcB*TTFC in the wild type and transformants.

Western blot analysis with specific polyclonal antibodies raised against the human IFN and bacterial TTFC proteins was used to further test the identity of the various protein bands shown in FIG. 19. FIG. 20, left panel, shows a strong positive cross-reaction between anti-IFN polyclonal antibodies and the CpcG1*IFN fusion protein at ˜46 kDa in all transformant lines tested. Moreover, cross-reactions were also detected with protein bands at a higher MW (>250 kDa) in the CpcB*TTFC+CpcG1*IFN samples, suggesting presence of higher order complexes containing the CpcG1*IFN fusion protein. FIG. 20, right panel, shows strong positive cross-reactions between anti-TTFC polyclonal antibodies and the CpcB*TTFC fusion protein at ˜73 kDa in all CpcB+TTFC+CpcG1*IFN double transformant strains tested. The ˜73 kDa size of these cross-reaction bands is consistent with the size of the CpcB*TTFC fusion protein. The higher MW bands (>250 kDa) likely originate from (α,β*TTFC)₃CpcG1*IFN undissociated dimer phycocyanin discs, which has a calculated MW of about at ˜316 kDa (please see below). Proteins from wild type cells did not show any cross-reactivity with either the anti-IFN or anti-TTFC antibodies, supporting the notion of high specificity of the respective immune sera. The above analyses suggested a correct assembly of functional heterohexameric (α,β*TTFC)₃CpcG1*IFN discs harboring the two fusion proteins, in quantities greater than 1% of the total cellular protein, consistent with the embodiments described above.

The above description illustrates that IFN can accumulate in Synechocystis in a fusion construct configuration with the CpcG1 linker protein. Further, IFN and TTFC can be co-expressed in quantities greater than 1% of the total cellular protein in Synechocystis, when placed in a double fusion CpcB*TTFC+CpcG1*IFN construct configuration.

### Column Affinity Chromatography and Native-PAGE Analysis of Proteins from CpcG1*IFN and Double Fusion CpcB*TTFC +CpcG1*IFN Transformants

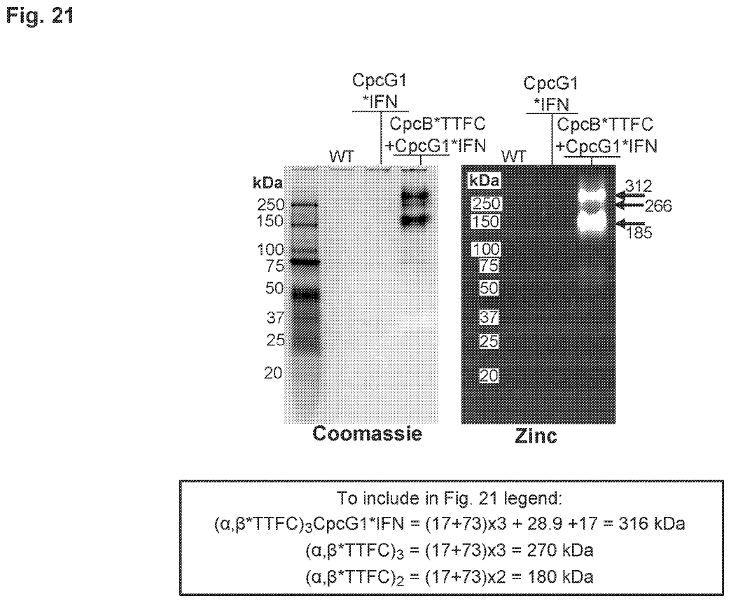

Total extracts from WT and transformant cells were investigated by cobalt affinity column chromatography and selective elution of the 6xHis-tagged fusion proteins. A comparison of the column affinity chromatography-eluted protein profiles of wild type, CpcG1*IFN fusion, and CpcB*TTFC+CpcG1*IFN double fusion is shown in the Native-PAGE results of FIG. 21.

The Coomassie stain (FIG. 21, left panel) of proteins eluted by the selective cobalt column affinity chromatography showed the presence of protein bands only from the CpcB*TTFC+CpcG1*IFN double transformant samples, whereas cellular proteins from the WT and the CpcG1*IFN transformant failed to be differentially eluted from the Co-column. This is understood for the wild type, as it does not contain the His-tag needed to bind it to the Co-column. The CpcB*TTFC transformant possesses the 6xHis tag, needed for differential adherence to the column, and eluted proteins are seen from the CpcB*TTFC+CpcG1*IFN double transformant extracts. It is more difficult to explain why CpcG1*IFN transformant failed to be differentially eluted from the Co-column, as this is also endowed with the 6xHis tag (see discussion below). It may reflect a tertiary configuration and folding of the CpcG1*S7*6xHis*tev*IFN fusion protein that results in a steric hindrance preventing the 6xHis-tag from accessing and binding to the f Co site in the column. In any case, Zinc-chromophore staining of the Native-PAGE (FIG. 21, right panel) corroborated the results from the Coomassie stain showing presence of protein complexes harboring phycocyanobilin, associated with the CpcB and CpcA subunits.

The Native-PAGE analysis showed the presence of three bands, clearly visible in the CpcB*TTFC+CpcG1*IFN double transformant extracts. The largest showed electrophoretic mobility calculated to be at about 312 kDa, corresponding to the expected MW of 316 kDa of the full size (α,β*TTFC)₃CpcG1*IFN complex. A smaller, and minor, protein band at ˜266 kDa matched closely the trimer configuration (α,β*TTFC)₃, which has an expected MW of 270 kDa. The lower band at ˜185 kDa could originate from a dimer (α,β*TTFC)₂ configuration, which has a MW of 185 kDa. These results showed retention of the heterohexameric (α,β*TTFC)₃CpcG1*IFN structure in Native-PAGE analysis, but also suggested a partial dissociation of the (α,β*TTFC)₃CpcG1*IFN complex, resulting in the appearance of lower than 316 kDa products.

The technical analysis presented in this section showed that column-eluted proteins from the single-transformant CpcB*TTFC crude extracts migrated as a band at ˜296 kDa. which electrophoretic mobility corresponds to the (α,β*TTFC)₃CpcG1 undissociated complex. Therefore, presence of the IFN protein as an additional fusion with the cpcG1 gene (CpcG1*IFN) would increase the size of the complex by about 19 kDa. In this respect, it is of interest that the entire double fusion (α,β*TTFC)₃CpcG1*IFN protein is in fact eluted as a single unit, signifying strong binding of the CpcG1*IFN linker to the (α,β*TTFC)₃ heterohexameric disc. The elution of this disc without the CpcG1*IFN, as well as of the band at ˜185 kDa, may be due to the fact that the complex was in different assembly stages in the cell at the time of protein extraction and purification or that some partial dissociation occurred during the cell lysis and related experimental manipulations described in this detailed technical description. The latter alternative gains credence due to the presence of Triton X-100 in the sample prior to column chromatography, as Triton was used to clarify the crude cell extracts (please Materials and methods section).

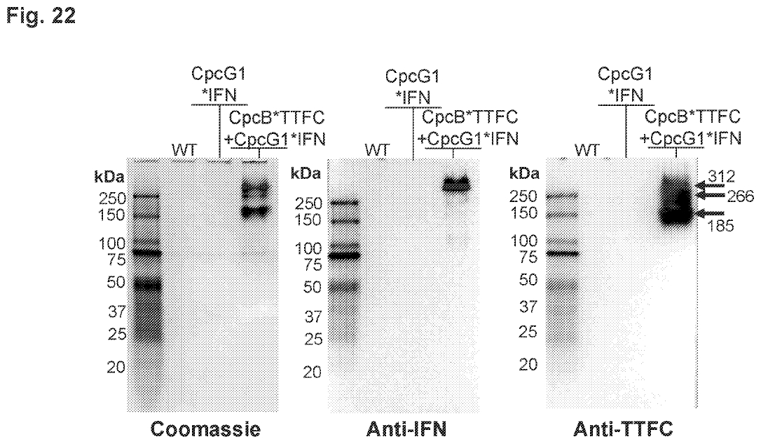

Western blot analysis with specific polyclonal antibodies raised against the human IFN and bacterial TTFC proteins was used to further test the protein identity of the bands in FIG. 21. Results are shown in FIG. 22, where that anti-IFN polyclonal antibodies specifically cross-reacted with the upper ˜312 kDa protein band (FIG. 22, center panel), whereas the anti-TTFC polyclonal antibodies (FIG. 22, right panel) specifically cross-reacted with all three protein bands (312, 266, and 185 kDa), as detected in the Native-PAGE analysis of the CpcB*TTFC+CpcG1*IFN eluted extracts. These results lend additional support to the interpretation that a 312 kDa band emanates from the (α,β*TTFC)₃CpcG1*IFN complex, whereas the (α,β*TTFC)₃˜266 kDa and (α,β*TTFC)₂˜185 kDa bands lack the CpcG1*IFN fusion and originate from the partial dissociation of the double fusion heterohexameric (α,β*TTFC)₃CpcG1*IFN complex.

### Absorbance Spectral Analysis of (α,β)₃CpcG1*IFN and Double Fusion (α,β*TTFC)₃CpcG1*IFN Mutants

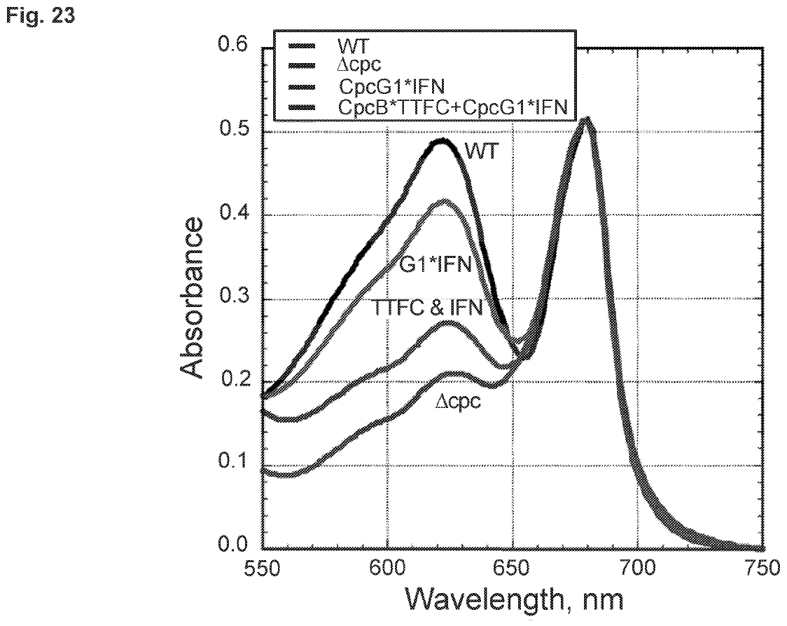

Absorbance spectra of lysed cell suspensions were measured to evaluate the pigment profile of (α,β)₃CpcG1*IFN and double fusion (α,β*TTFC)₃+CpcG1*IFN relative to that of the wild type and Δcpc strains (FIG. 23). The absorbance spectra were normalized to the chlorophyll max at 678 nm so that differences of the absorbance at 620 nm, where phycocyanin absorbs, could be more easily assessed among the various strains. WT cells showed the typical absorbance bands of chlorophyll a peaking at 678 nm and phycocyanin at 620 nm. The Δcpc strain (FIG. 17D) lacked the entire cpc operon genes and, therefore, lacked the CpcB and CpcA phycocyanin proteins due to the cpc operon deletion. The absorbance spectrum of the Δcpc strain showed a low amplitude peak at 620 nm, which is attributed to a chlorophyll a secondary absorbance in this spectral region.

The absorbance spectra of the (α,β*TTFC)₃CpcG1*IFN double fusion mutant showed an elevated 620 nm band (FIG. 23). The ratio of phycocyanin absorbance of the double fusion (α,β*TTFC)₃CpcG1*IFN to that of the wild type from four independent biological replicates was measured as A(620 nm,CpcB*TTFC+G1*IFN)/A(620 nm,WT)=11.5%±5.0, i.e., 1 to 8.7 mutant to wild type phycocyanin ratio.

The absorbance spectra of the (α,β)₃CpcG1*IFN (FIG. 17B) unexpectedly showed a substantially elevated absorbance at 620 nm (FIG. 23), suggesting the presence of considerable amounts of phycocyanobilin chromophore and phycocyanin pigment in these strains. The ratio of phycocyanin absorbance of the (α,β)₃CpcG1*IFN transformants to that of the wild type from four independent biological replicates was A(620 nm,G1*IFN)/A(620 nm,WT)=68.4%±4.0, i.e., presence of about two thirds of the wild type phycocyanin in this mutant.

To better understand the organization of the extra phycocyanin in the (α,β)₃G1*IFN mutant, mass spectrometry sequencing analysis was undertaken of protein bands excised from the 25-37 kDa SDS-PAGE electrophoretic migration position. This effort sought to investigate whether additional phycocyanin linkers are present in the examined transformants. SDS-PAGE was loaded with the same amount of total protein extract from the strains denoted as WT, CpcG1*IFN and CpcB*IFN+CpcG1*IFN. The qualitative MS analysis (Table 5) consistently showed presence of the phycocyanin 32 kDa linker polypeptide CpcC1 in all strains examined, and absence of the 30 kDa CpcC2 linker polypeptide in either of the two transformants (Table 5). The CpcC1 linker functions to provide structural stability to the middle disc in the phycocyanin rod structure, while CpcC2linker is associated with the distal disc in the phycocyanin rod. These results can be explained to indicate presence of both the proximal and middle phycocyanin discs, along with their respective linker polypeptides, and absence of the distal disc in the CpcG1*IFN mutant.

### Functional Antenna Analysis of the (α,β)₃CpcG1*IFN and Double Fusion (α,β*TTFC)₃CpcG1*IFN Complexes

The functional association of the (α,β)₃CpcG1*IFN and double fusion (α,β*TTFC)₃CpcG1*IFN heterohexameric complexes as a residual phycocyanin antenna in the transformants was investigated by sensitive absorbance spectrophotometry, measuring the functional PSII absorption cross section to 620 nm light, where phycocyanin absorbs, and from the yield Φ of chlorophyll a fluorescence of intact and DCMU-inhibited Synechocystis. Cells were suspended in a 1.5 mm pathlength spectrophotometer cuvette in the range of 22 to 28 μg Chl mL⁻¹ (Table 6). Weak actinic excitation at 10 μmol photons m⁻² s⁻¹ was provided at 619.5 nm by a narrow-bandpass Baird Atomic interference filter coupled with a 659.6 nm visible bandpass negative cut-off Ealing filter.

The rate constant k_{II} of PSII photochemistry was measured from the fluorescence induction kinetics of intact cells suspended in the presence of 12 μM DCMU (Table 6) (Melis and Duysens 1978; Melis 1989). Under weak actinic excitation, rates of PSII photochemistry are directly proportional to the number of pigment molecules acting as light-harvesting antennae for this photosystem. Under these conditions, k_{II} values show the rate constants (rates of light absorption) obtained upon illumination of dark-adapted cells. It is evident from the k_{II} (s⁻¹) results that k_{II}-(G1*IFN) and k_{II}-(CpcB*TTFC+G1*IFN) are greater than k_{II}-(Δcpc), but lower than that of the k_{II}-(WT) (Table 6, k_{II}-s⁻¹ column).

A measure of the direct phycocyanin contribution to the rate of photochemistry was obtained upon subtracting the contribution of chlorophyll a in the Δcpc k_{II} from the k_{II} value of the transformants and from that of the wild type. This is presented in Table 6, column k_{II} s⁻¹ (minus the k_{II} of Δcpc). This analysis showed that phycocyanin in the (α,β)₃CpcG1*IFN mutant transferred excitation energy to PSII at a 22.4% rate, when compared to phycocyanin in the wild type (average k_{II} of 4.12 s⁻¹ versus 18.37 s⁻¹, respectively). Here, we noted a quantitative discrepancy between phycocyanin content and photochemistry, as the (α,β)₃ CpcG1*IFN mutant contained 68.4% of the wild type phycocyanin (FIG. 23) but it apparently contributes only 22.4% excitation energy transfer to PSII.

Conversely, phycocyanin in the (α,β*TTFC)₃CpcG1*IFN double mutant transferred excitation energy to PSII at a 13.4% rate, when compared to phycocyanin in the wild type (average k_{II} of 2.47 s⁻¹ versus 18.37 s⁻¹, respectively). This is more closely in agreement with the relative phycocyanin content of 11.5%, noted from the absorbance spectra in this mutant.

The raw chlorophyll a fluorescence yield Φ of the various strains is shown in Table 6 (Fluorescence yield). The yield data normalized to that of chlorophyll loaded in the cuvette and, in parentheses, further normalized to that of the Δcpc strain (=1.0) are shown in Table 6 (Fluorescence yield normalized to Chl). On average, Φ((α,β)₃G1*IFN) was about 1.55× greater than Φ(Δcpc). Φ((α,β*TTFC)₃G1*IFN) from the double mutant was about 1.45× greater than Φ(Δcpc). These results are qualitatively consistent with the k_{II} s⁻¹ measurements and suggested that, under these experimental conditions, higher rates of excitation energy arrived at PSII in the fusion transformants than in the Δcpc, presumably because more actinic light was harvested (greater antenna size) in the latter than in the Δcpc strain. Φ(wild type) was 2.5× greater than (Δcpc) (Table 6).

It is noted that the CpcB*TTFC+G1*IFN double mutant, A(620 nm), k_{II} , and the relative Φ, for are consistent with each other. However, in the (α,β)₃CpcG1*IFN mutant, A(620 nm) is far greater than k_{II}, and the relative Φ. This observation is further discussed below.

### Analysis of Technical Results

The embodiments described in Example 2 illustrate that independent recombinant proteins are over-expressed either by the CpcG1 linker fusion construct or by the CpcA phycocyanin fusion construct approach. An important requirement for the substantial accumulation of recombinant proteins in cyanobacteria, and avoidance of degradation of heterologous proteins by the cellular proteasome is a need by the cell to functionally benefit from the non-native structure (Zhang et al. 2021). In the present technical description. substantial and stable accumulation (10.7% of total cellular protein, FIGS. 19, 20) of recombinant proteins such as TTFC as a fusion with the CpcB β-subunit was demonstrated as well as substantial and stable accumulations of IFN as a fusion with the CpcA and CpcG1 (6.4% and 3% of total cellular protein, respectively, FIGS. 14, 15). The overexpression of these recombinant proteins formed a functional protein complex for the cell, which complex performs light-harvesting and excitation energy transfer. Not to be bound by theory, the cell tolerates TTFC and IFN exogenous proteins so long as they are part of a useful functional complex, which is required for competition and survival. In this case. TTFC and IFN, which play no functional role in the Synechocystis physiology, accumulated in direct proportion to the assembled CpcB and CpcG1 proteins, respectively. The latter form a rudimentary, under these conditions, functional light-harvesting complex in the form of a phycocyanin (α,β*TTFC)₃CpcG1*IFN disc. Thus, this technical description demonstrated that overexpression, in an SDS-PAGE Coomassie-stain visible amount, can be obtained of two proteins in close proximity to each other that are, by their origin, foreign to the cell.

Earlier reports showed that such fusion constructs retain the activity of the recombinant protein, as the case was for IFN (Betterle et al. 2020) and enzymes such as the isoprene and β-phellandrene synthases (Chaves et al. 2017; Formighieri and Melis 2016; Betterle and Melis 2019). The technical description provided in this disclosure thus demonstrates robust and stable expression using a fusion construct approach, which can be used as a platform for expression of recombinant target proteins of interest.

In wild type Synechocystis, the light-harvesting phycobilisome complexes include the allophycocyanin-containing core cylinder and the phycocyanin-containing peripheral rods. There are three core cylinders in Synechocystis, the long axis of which is parallel to the surface of the thylakoid membrane. Two of the core cylinders rest directly on top of the thylakoid membrane at the PSII dimer locus, while the third is resting in parallel and on top of the first two (Kirst et al. 2014). Wild type phycobilisomes in Synechocystis have six phycocyanin peripheral rods, which emanate radially from the core cylinders and are exposed to the aqueous medium of the cellular cytosol. Peripheral rods are made of (α,β)₃ heterohexameric disc units, organized in three (α,β)₃(α,β)₃ heterohexameric dimers. These are stacked on each other with the proximal (P) (α,β)₃(α,β)₃ disc tethered onto the core cylinders via the cpcG1 gene product, whereas the middle (M), and distal (D) (α,β)₃(α,β)₃ phycocyanin dimer discs are placed away from the core. A distinction between proximal (P), middle (M), and distal (D) (α,β)₃(α,β)₃ phycocyanin dimer discs is realized by the placement of linker polypeptides, which occupy the hollow channel in the center of the (α,β)₃(α,β)₃ phycocyanin dimer discs, thereby ensuring structural and functional integrity of the phycocyanin rods. Phycocyanin in the PC discs that are proximal to the AP core cylinders structurally and electronically couple to the core allophycocyanin through the colorless CpcG1 polypeptide linkers (De Marsac and Cohen-Bazire 1977; Ughy and Ajlani 2004; Kondo et al. 2005). Additional colorless linker polypeptides, e.g., the cpcC1 and cpcC2 gene products, ensure the structural stability of the middle (M) and distal (D) discs in the PC rods, respectively (Yamanaka et al. 1978; 1982; Ughy and Ajlani 2004). A recent structural study (doi. available at doi.org/10.1101/2021.11.15.468712) suggested that the C-terminus of the CpcG1 protein extends through the hollow channel of the proximal (α,β)₃(α,β)₃ phycocyanin dimer disc, such that about 60 amino acid residues of the CpcG1 C-terminus extension attach it, and the proximal (α,β)₃(α,β)₃ phycocyanin dimer, to the allophycocyanin core.

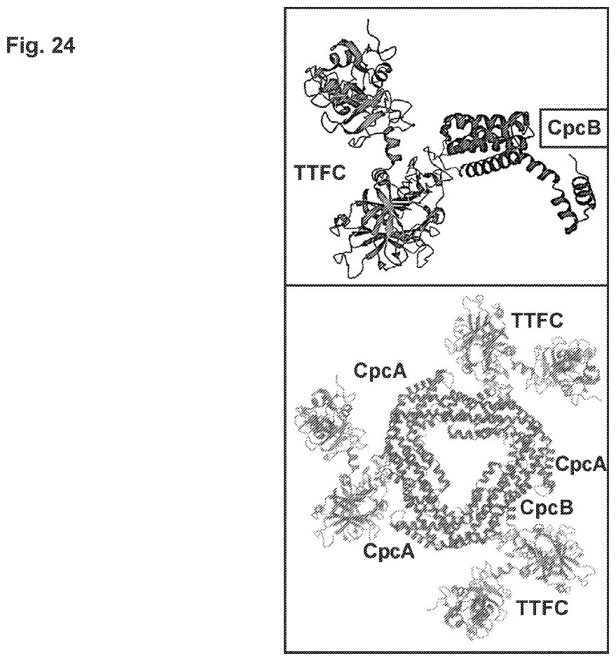

Results obtained with respect to the the technical embodiments described herein suggested that the middle (M), and distal (D) (α,β)₃(α,β)₃ phycocyanin dimer discs are not retained in the CpcB*TTFC fusion constructs, as only a single proximal (P) (α,β*TTFC); heterohexamer disc was detected, i.e., about one sixth of the full size phycocyanin rods. It was concluded that presence of the TTFC protein as a fusion with the C-terminus of the CpcB β-subunit of phycocyanin (α,β*TTFC)₃ prevented the assembly of additional (α,β)₃ heterohexamers due to space constrains. A folding model depiction of the CpcB*TTFC fusion protein is shown in FIG. 24, upper panel, whereas the folding model of the assembled solitary (α,β*TTFC)₃ phycocyanin fusion disc is shown in FIG. 24, lower panel Noted is the peripheral placement and radial orientation of the recombinant TTFC with respect to the (α,β)₃ compact disc. The double fusion (α,β*TTFC)₃CpcG1*IFN embodiment described herein also showed this single-disc limitation in both assembly and function of phycocyanin, with phycocyanobilin pigment content and rate of excitation energy transfer to PSII being equal to about one eighth of that measured with the wild type. This analysis suggested that phycocyanin assembly in the double fusion (α,β*TTFC)₃ CpcG1*IFN mutant does not exceed the single heterohexameric disc per phycocyanin rod.

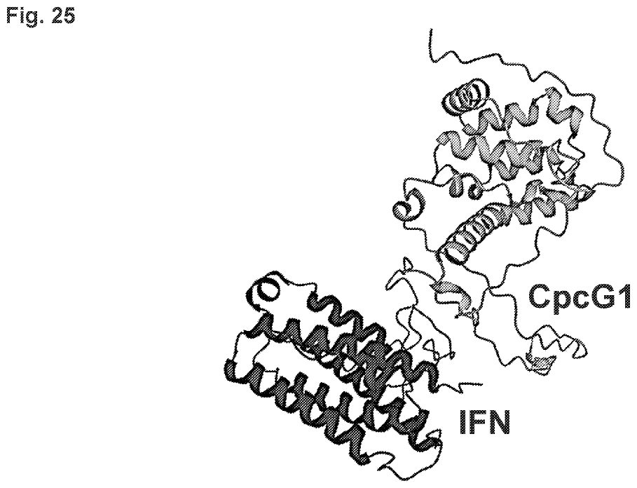

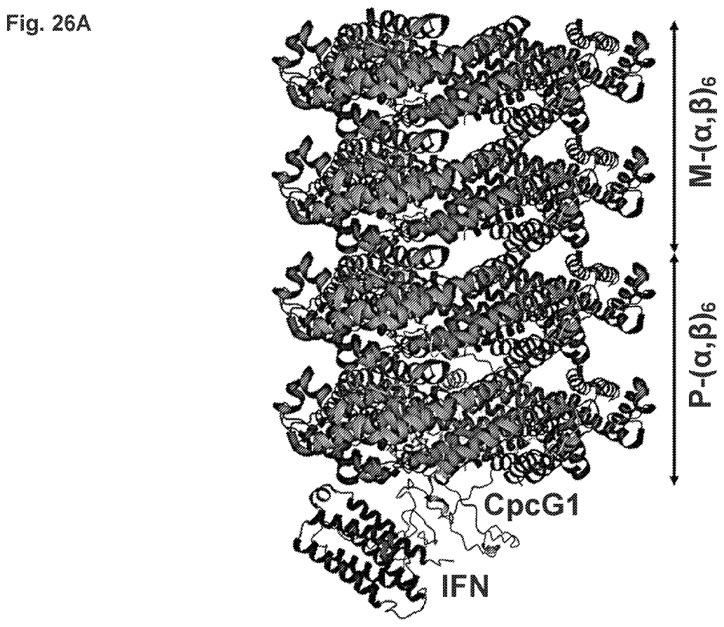

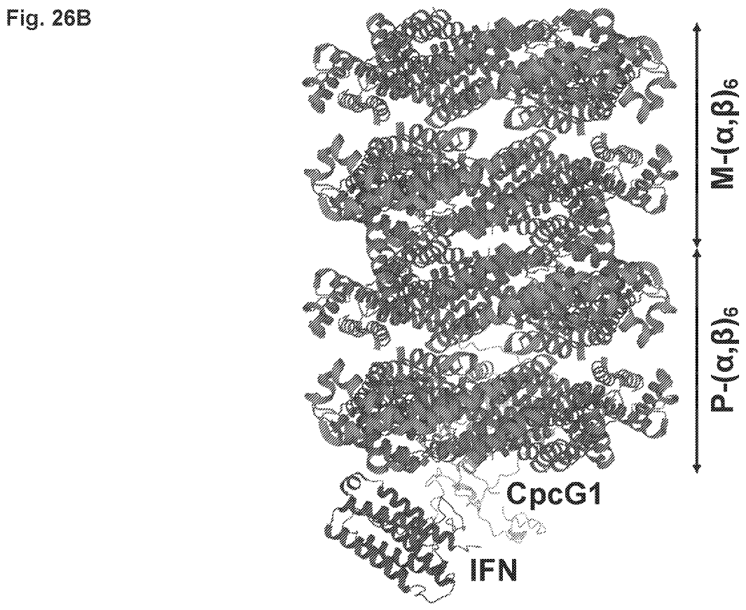

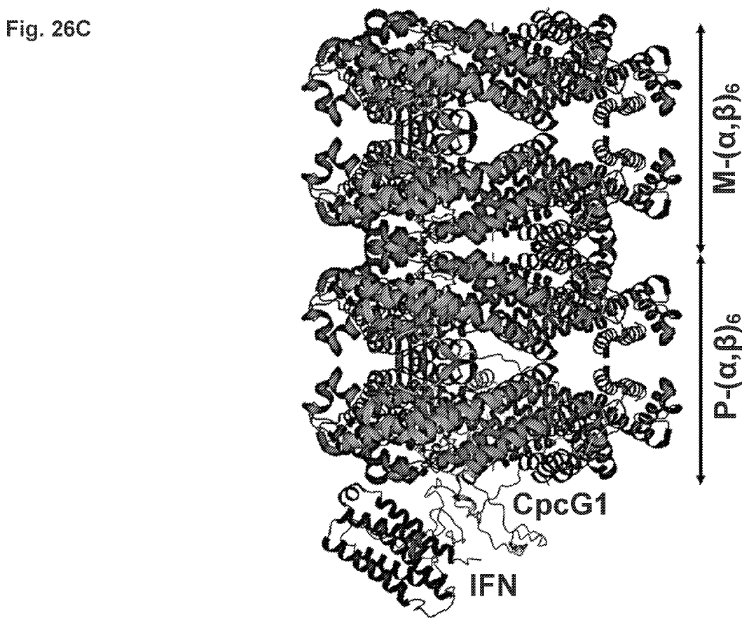

Surprisingly, the phycocyanin rod configuration of the simpler (α,β)₃CpcG1*IFN fusion construct was substantially different from that described above for the (α,β*TTFC)₃CpcG1*IFN double fusion construct. The (α,β)₃CpcG1*IFN assembled about two thirds of the wild type phycocyanin (68.4% of the WT phycocyanin), signifying the assembly of the proximal and middle (α,β)₃(α,β)₃ phycocyanin dimer discs, presumably with the CpcG1*IFN linker in association with the proximal (α,β)₃(α,β)₃ dimer disc and the CpcC1 linker in association with the middle (α,β)₃(α,β)₃ phycocyanin dimer disc. FIG. 25 shows a folding model of the CpcG1*IFN fusion protein with the CpcG1 linker in the leader and IFN in the trailing protein position in this construct. FIG. 26A shows the likely folding model of the proximal P-(α,β)₃CpcG1*IFN(α,β)₃ and middle M-(α,β)₃(α,β)₃ heterobexamer dimer structures. Inferred from this structure is the likelihood of IFN interference in the binding interaction between the exposed portion of the CpcG1 and the core cylinders of the Synechocystis phycobilisome. Such interference may explain the reason why the (α,β)₃CpcG1*IFN fusion mutants possess 68.4% of the wild type phycocyanin but are able to contribute only 22.4% to the rate of PSII photochemistry, when compared with that of the wild type (Table 6, see rates of 4.12 s⁻¹ for the (α,β)₃CpcG1*IFN mutant versus 18.37 s⁻¹ for the wild type). This is also reflected in the chlorophyll a fluorescence yield data, whereby the Φ((α,β)₃G1*IFN) is marginally greater than that of the Φ((α,β*TTFC)₃G1*IFN), and substantially lower of that in the Φ(wild type). Not to be bound by theory, one explanation of this functional inhibition is that the protruding IFN protein increased the distance or altered the conformation relationship between the terminal phycocyanobilin in the phycocyanin rods and the receiving allophycocyanin chromophore in the core cylinders, thereby impeding excitation energy transfer from the phycocyanin rods in the (α,β*TTFC)₃CpcG1*IFN strain to allophycocyanin and PSII.

### 3. IFN*cpcA and IFN*cpcG1 Fusion Constructs

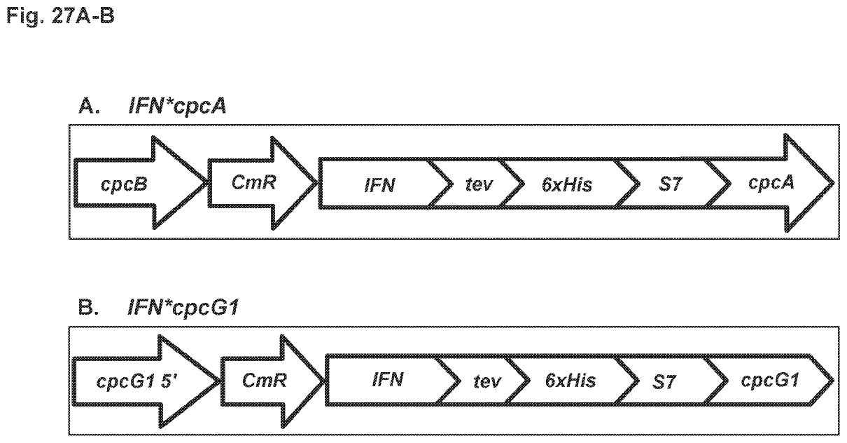

This section describes design of IFN*cpcA and IFN*cpcG1 fusion constructs (FIG. 27) and demonstrates target protein over-expression exceeding the 1% of the total cellular protein content.

### Design of Fusion Constructs IFN*cpcA

Relative to the wild type cpc operon in Synechocystis (FIG. 13A), a DNA construct was designed, comprising a fusion between the human interferon α-2 (IFN) and the α-subunit of phycocyanin cpcA gene (FIG. 27A). In this case, the IFN was placed in the leader sequence position, whereas the CpcA was in the trailing protein position (IFN C-terminus to CpcA N-terminus fusion). The IFN and cpcA sequences in the IFN*cpcA fusion construct (FIG. 27A) were separated with short pieces of DNA encoding the Tobacco Etch Virus Protease cleavage site (tev) to facilitate in vitro enzymatic cleaving of the leader and trailing proteins (Zhang et al. 2021), the His tag (6xHis) DNA to enable a differential column affinity chromatography isolation of the fusion construct proteins, and a seven amino acid spacer (S7) in order to further distance the two proteins. The latter enabled a stretching between the IFN and CpcA proteins, conferring the tertiary configuration needed for the TEV enzyme to access the tev amino acid sequence and, thus, to facilitate cleaving of the two proteins, thereby releasing a native form of the target (IFN) enzyme (Zhang et al. 2021). These spacer additions did not interfere with the over-expression of phycocyanin subunit fusion constructs (Betterle et al 2020). This IFN*cpcA fusion construct was preceded by the chloramphenicol (cmR) resistance cassette in an operon configuration. The nucleotide and amino acid sequences of this cmR-IFN*tev*6xHis*S7*cpcA construct are provided in the Illustrative Expression Constructs and Sequences section below. Following transformation and antibiotic selection. attainment of transgenic DNA copy homoplasmy in the transformant strains was evidenced through genomic DNA PCR analysis (not shown).

### Design of Fusion Constructs IFN*cpcG1

A DNA construct comprising a fusion between the IFN and cpcG1 genes is shown in FIG. 27B, (IFN*cpcG1). Sequence information for this construct is provided in the Illustrative Expression Constructs and Sequences section below. The native to Synechocystis cpcG1 gene, which encodes the colorless CpcG1 28.9 kDa linker polypeptide, was replaced with fusion construct cmR-IFN*tev*6xHis**S7*cpcG1 (referred to as IFN*cpcG1). In this construct, the IFN was placed in the leader sequence position, whereas the CpcG1 was in the trailing protein position (fusion of the IFN C-terminus to CpcG1 N-terminus). The CpcG1 polypeptide serves to anchor the proximal phycocyanin (α,β)₃(α,β)₃ dimer disk to the phycobilisome core cylinders. The IFN*cpcG1 fusion DNA was preceded by the chloramphenicol (cmR) resistance cassette in an operon configuration. Following transformation and antibiotic selection, attainment of transgenic DNA copy homoplasmy in the transformant strains was tested through genomic DNA PCR analysis (not shown).

### Protein Analysis of Total Cell Extracts from the IFN*cpcA Fusion Transformants

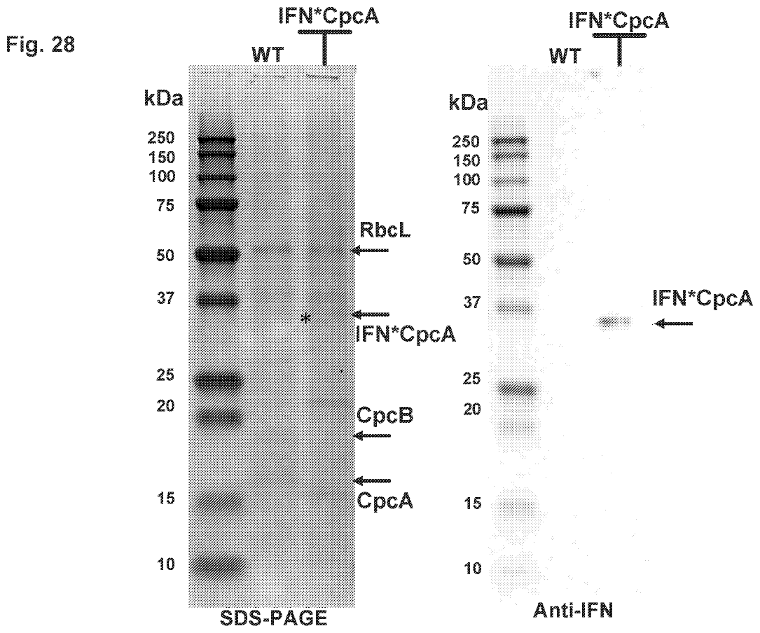

A combined approach to protein analysis from WT and IFN*cpcA fusion transformants of Synechocystis was implemented through SDS-PAGE followed by Coomassie blue staining, and Western blot analysis (FIG. 28).

FIG. 28 (left panel) shows the dominant presence of the CpcB β-subunit and CpcA α-subunit of phycocyanin in the WT, migrating to ˜19 and ˜17 kDa, respectively. The IFN*CpcA transformant strains lacked the individual ˜19 CpcB and ˜17 kDa CpcA proteins and showed, instead, a new protein band migrating to ˜34 kDa, attributed to the IFN*CpcA fusion protein. FIG. 28, left panel, also shows the electrophoretic mobility of RbcL, the large subunit of Rubisco, migrating to about 56 kDa, which was present in both samples. Quantitative gel scanning measurements showed that the ˜34 kDa CpcA*IFN fusion protein accounted for 3-5% of the total cellular protein, as measured from the Coomassie blue staining of the bands.

Western blot analysis with specific polyclonal antibodies raised against the human IFN protein was used to further test the identity of the various protein bands shown in FIG. 28. FIG. 28, right panel, shows a positive cross-reaction between anti-IFN polyclonal antibodies and the IFN*CpcA fusion protein at ˜34 kDa in all transformant lines tested. Resolved proteins from wild type cells did not show any cross-reactivity with the anti-IFN antibodies, supporting the notion of high specificity of the anti-IFN immune sera.

### Protein Analysis of Total Cell Extracts from the IFN*cpcG1 Fusion Transformants

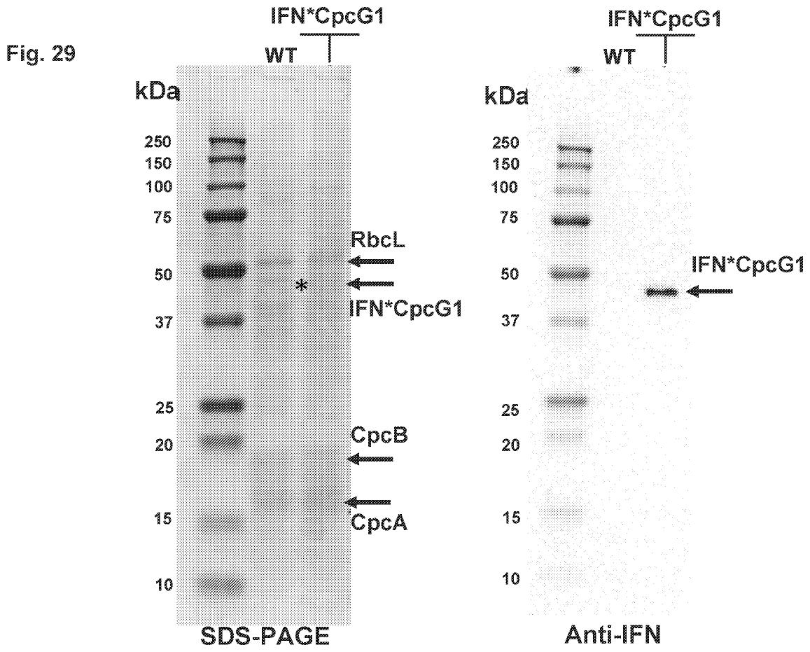

A combined approach to protein analysis from WT and IFN*cpcG1 fusion transformants of Synechocystis was implemented through SDS-PAGE followed by Coomassie blue staining, and Western blot analysis (FIG. 29).

FIG. 29 (left panel) shows the dominant presence of the CpcB β-subunit and CpcA α-subunit of phycocyanin, migrating to ˜19 and ˜17 kDa, respectively, in both wild type and transformant strains. The IFN*CpcG1 transformant strains showed a new protein band migrating to ˜46 kDa, attributed to the IFN*CpcG1 fusion protein. FIG. 29, left panel, also shows the electrophoretic mobility of RbcL, the large subunit of Rubisco, migrating to about 56 kDa, which was present in both samples. Quantitative gel scanning measurements showed that the ˜46 kDa IFN*CpcG1 fusion protein accounted for 3-5% of the total cellular protein, as measured from the Coomassie blue staining of the bands.

Western blot analysis with specific polyclonal antibodies raised against the human IFN protein was used to further test the identity of the various protein bands shown in FIG. 29. FIG. 29, right panel, shows a positive cross-reaction between anti-IFN polyclonal antibodies and the IFN*CpcG1 fusion protein at ˜46 kDa in all transformant lines tested.

Resolved proteins from wild type cells did not show any cross-reactivity with the anti-IFN antibodies, supporting the notion of high specificity of the anti-IFN immune sera.

### Abbreviations

AP: Allophycocyanin
PC: phycocyanin
cpcA: gene encoding the phycocyanin α-subunit
cpcB: gene encoding the phycocyanin β-subunit
cpcG: gene encoding the proximal phycocyanin linker protein CpcG1/CpcG2
Synechocystis: Synechocystis sp. PCC 6803
PBS: phycobilisome
PSII: photosystem II
Chl: Chlorophyll
P: protein of interest to be expressed
WT: wild type

### Detailed Description of Methods, Section 1 Above

Strains, Recombinant Constructs, and Culture Conditions. The unicellular cyanobacterium Synechocystis sp. PCC 6803 (Synechocystis) wilt type (WT) was used as the reference strain for the technical work described above. Transformants cpcB*6xHis*tev*IFN (abbreviated as CpcB*IFN), cpcB*6xHis*tev*TTFC (CpcB*TTFC) and Acpc have been described in recent work from this lab (Betterle et al 2020; Zhang et al 2021; Kirst et al. 2014). The generation of transformant cpcB*6xHis*tev (abbreviated as CpcB*) was generated by deletion of the tetanus toxic fragment C sequence (TTFC) gene from the CpcB*TTFC construct via the Q5 Site-Directed Mutagenesis Kit (New England Biolabs) and by use of primers Δttfc_fw (5′-TGAGGAATTAGGAGGTAATATATG-3′) and Δttfc_rv (5′-GCCTTGTAAATACAAATTATCATG-3′).

Transformation of Synechocystis was performed according to protocols earlier described (Williams 1988; Eaton-Rye 2011; Lindberg et al. 2010). All strains were maintained on BG11 media supplemented with 1% agar, 10 mM TES-NaOH (pH 8.2), 0.3% sodium thiosulfate and the corresponding antibiotic (20 mg L⁻¹ chloramphenicol, 15 mg L⁻¹ spectinomycin, or 10 mg L⁻ kanamycin). Cell suspensions in liquid culture were cultivated in 1 L bottles, buffered with 37.5 mM sodium bicarbonate and 6.25 mM dipotassium hydrogen phosphate (pH 9), instead of TES buffer, and incubated in the light with continuous gentle agitation. Illumination was provided with a balanced combination of white LED and incandescent light bulbs to yield a final photosynthetically active radiation (PAR) intensity of ˜100 μmol photons m⁻² s⁻¹.

Genomic DNA PCR Analysis and Homoplasmy Testing. Synechocystis genomic DNA was extracted and prepared as described (Formighieri and Melis 2014). Briefly, 10 μL of cell suspension from a culture in the intermediate exponential growth phase (OD730-1) was mixed with 10 μL of 100% ethanol, then 100 μL of a 10% (w/v) Chelex 100 Resin (BioRad, Hercules, CA) were added. This mix was incubated at 98° C. for 10 min, followed by centrifugation at 16,000 g for 10 min. Two and a half microliters of the supernatant were used as a template in a 12.5 μL PCR reaction. Q5 High-Fidelity 2X Master Mix (New England Biolabs, Ipswich, MA) was used to perform the analysis. The state of genomic DNA homoplasmy was tested after a few rounds of selection with the appropriate antibiotic. The primers used for this test were cpcB_fw (5′-TGACATGGAAATCATCCTCC-3′) and cpcA_rv (5′-GGTGGAAACGGCTTCAGTTAAAG-3′). The location of these primers on the DNA constructs are indicated in FIG. 1 with forward and reverse arrows for the respective DNA constructs.

Protein extraction, purification and electrophoresis. Fifty mL of cells suspension in the intermediate exponential growth phase (OD730˜1) were pelleted by centrifugation at 4,500 g for 5 min. The cells were suspended in 2 mL of a solution buffered with 50 mM Tris-HCl, pH 8.2 supplemented with a cOmplete™ mini protease inhibitor cocktail (Roche) and kept on ice. Then, cells were broken by passing the suspension through a French press cell at 1,500 psi. The unbroken cells were removed by slow speed centrifugation at 350 g for 3 min. The supernatant with the crude cell extracts were kept on ice until use or at −80° C. for long term storage.

Recombinant protein purification was performed using 400 μL of total crude cellular extracts mixed with 1 M HEPES buffer, pH=7.5, and Triton X-100 to yield final concentrations of 20 mM and 0.2%, respectively. This mix was incubated at room temperature for 20 min with gentle shaking. After this incubation, samples were centrifugated for 5 min at 16,000 g to remove cell debris and insoluble material. The supernatant was mixed with 100 μL of HIS-Select® Cobalt Affinity Gel (Sigma-Aldrich. St. Louis, MO, United States) for the cobalt affinity chromatography. Selective elution of the fusion proteins was performed according to the manufacture's recommendations.

Samples for denatured electrophoretic analysis of proteins (SDS-PAGE) were solubilized for 30 min at room temperature in the presence of 1× Laemmli Sample Buffer (BioRad, Hercules, CA), supplemented with a final concentration of 1 M urea and 5% β-mercaptoethanol. The samples were briefly vortexed every 10 min to enhance solubilization. Prior to loading onto SDS-PAGE, samples were centrifuged at 16,000 g for 3 min to remove cell debris and insoluble material. Samples for native PAGE analysis were just mixed with equal parts of 2× loading buffer (62.5 mM Tris-HCl, pH 6.8, 40% glycerol, 0.01% bromophenol blue) prior to loading the PAGE lanes. The SDS-PAGE and Native-PAGE were performed with a lane load of 20 μl, using the 12-well Any kD™ Mini-PROTEAN® TG™ Precast Protein Gels. (BioRad, Hercules, CA). Densitometric analysis of protein bands was performed using the BioRad (Hercules, CA) Image Lab software.

Zinc and Coomassie staining. SDS-PAGE or Native-PAGE were incubated in the presence of 5 mM zine sulfate for 30 min (Li et al. 2016; Betterle et al. 2020) To detect covalent chromophore-binding polypeptides, zinc-induced fluorescence was measured by the Chemidoc imaging system (BIORAD), employing UV irradiance as a light source. After registering the Zn-chromophore fluorescence, gels were incubated overnight in a solution of 0.1% Coomassie Blue G, 37% methanol, 3% phosphoric acid, and 17% ammonium sulfate. Finally, gels were washed with 5% acetic acid to remove excess Coomassie stain.

Protein analysis by mass spectrometry. Mass spectrometry was performed by the Vincent J. Coates Proteomics/Mass Spectrometry Laboratory at UC Berkeley. Sample preparation was performed according to internal protocols of the Vincent J. Coates Lab. In brief, digestion of proteins in SDS-PAGE slices consisted of washing the gel pieces for 20 min in 100 mM NH₄HCO₃. After discarding the first wash, an incubation at 50° C. with 100 mM NH₄HCO₃ and 45 mM DTT was done for 15 min. To the cooled down mix 100 mM iodoacetamide were added and incubated in the dark for 15 min. Then, the solvent was discarded and the gel slice was washed with 50:50 mix of acetonitrile and 100 mM NH₄HCO₃ with shaking for 20 min. The wash was repeated just with acetonitrile, followed by drying the gel fragments in a speed vac. The gel pieces were rinsed thoroughly with 25 mM NH₄HCO₃ containing Promega modified trypsin and incubated for 8 h at 37° C. The supernatant was removed and placed in new microcentrifuge tubes. To extract remaining peptides, the gel pieces were treated by adding 60% acetonitrile and 0.1% formic acid for 20 min, then once with acetonitrile. Finally, the supernatant was subjected to speed vac to dryness. Fusion proteins from the cobalt affinity chromatography and selective elution were buffer exchanged with 8 M Urea and 100 mM Tris-HCl, pH 8.5. prior to been treated with the reducing, alkylating agent and the corresponding digestion steps mentioned above.

A nano LC column was packed in a 100 μm inner diameter glass capillary with an emitter tip. The column consisted of 10 cm of Polaris c18 5 μm packing material. The column was loaded by use of a pressure bomb and washed extensively with buffer A solution (see below). The column was then directly coupled to an electrospray ionization source mounted on a Thermo-Fisher LTQ XL linear ion trap mass spectrometer. An Agilent 1200 HPLC equipped with a split line so as to deliver a flow rate of 300 nL min⁻¹ was used for chromatography. Peptides were eluted with a 90 minus gradient to 60% B. Buffer A contained 5% acetonitrile and 0.02% beptaflurobutyric acid (HBFA). Buffer B contained 80% acetonitrile and 0.02% HBFA.

Protein identification was done with Integrated Proteomics Pipeline (IP2, Integrated Proteomics Applications, Inc. San Diego, CA) using ProLuCID/Sequest, DTASelect2 and Census (Xu et al 2006, Cociorva et al 2007, Tabb et al 2002, Park and Venable 2008). Tandem mass spectra were extracted into msl and ms2 files from raw files using RawExtractor (McDonald et al 2004). Data were searched against a database of Synechocystis sp. PCC6803 downloaded for Uniprot in December 2020 and supplemented with sequences of possible common contaminants. The database was concatenated to a decoy database in which the sequence for each entry in the original database was reversed (Peng et al 2003). LTQ data was searched with 3000.0 milli-amu precursor tolerance and the fragment ions were restricted to a 600.0 ppm tolerance. All searches were parallelized and searched on the VJC proteomics cluster. Search space included all fully tryptic peptide candidates with no missed cleavage restrictions. Carbamidomethylation (+57.02146) of cysteine was considered a static modification. We required 1 peptide per protein and both tryptic termini for each peptide identification. The ProLuCID search results were assembled and filtered using the DTASelect program (Cociorva et al 2007, Tabb et al 2002) with a peptide false discovery rate (FDR) of 0.001 for single peptide and a peptide FDR of 0.005 for additional peptides of the same protein.

Photosystem II absorption cross section measurements Rates of light absorption and the associated effective light-harvesting antenna size of photosystem-II in the various Synechocystis transformants were measured from the chlorophyll fluorescence induction kinetics of cells suspended in the presence of 12 or 24 μM 3-(3,4-dichlorophenyl)-1,1-dimethylurea (DCMU)-treated cells, as previously described (Melis 1989). Weak actinic excitation (10 μmol photons m⁻² s⁻¹) was defined at 619.5 nm by a narrow-bandpass Baird Atomic interference filter coupled with a 659.6 nm visible bandpass negative cut-off Ealing filter. Chlorophyll fluorescence emission was recorded at 700 nm, defined by a 700 nm narrow-bandpass Baird Atomic interference filter coupled with a 695 nm red cut-off Schott filter. The rate constant of light absorption by PSII was measured from the slope of the straight line, following a first-order kinetic analysis of the area accumulation over the variable fluorescence induction curve. The latter is a direct measure the kinetics of QA photoreduction under these experimental conditions (Melis and Duysens 1978).

### References for Citations Cited by Author and Year of Publication

Arteni AA. Ajlani G, Boekema EJ (2009) Structural organisation of phycobilisomes from Synechocystis sp strain PCC6803 and their interaction with the membrane. Biochim Biophys Acta-Bioenerg 1787(4):272-279.
Baier A, Winkler W, Korte T, Lockau W. Karradt A. (2014) Degradation of phycobilisomes in Synechocystis sp. pcc6803 evidence for essential formation of an nbla1/nbla2 heterodimer and its codegradation by a clp protease complex. J Biol Chem. 289(17):11755-66
Baier. T., Kros. D., Feiner, R. C., Lauersen, K. J., Müller, K. M., and Kruse, O. (2018) Engineered fusion proteins for efficient protein secretion and purification of a human growth factor from the green microalga Chlamydomonas reinhardni. ACS Synth. Biol. 7,2547-2557
Betterle N, Hidalgo Martinez DA, Melis A (2020) Cyanobacterial production of biopharmaceutical and biotherapeutic proteins. Front. Plant Sci. 11:237.
Betterle N. Melis A (2018) Heterologous leader sequences in fusion constructs enhance expression of geranyl diphosphate synthase and yield of β-phellandrene production in cyanobacteria (Synechocystis) ACS Synth Biol 7:912-921
Betterle N, Melis A (2019) Photosynthetic generation of heterologous terpenoids in cyanobacteria. DOI: Biotechnology and Bioengineering 116:2041-2051. 10.1002/bit.26988
Bolte K, Kawach O, Prechtl J, Gruenheit N, Nyalwidhe J, Maier UG (2008) Complementation of a phycocyanin-bilin lyase from Synechocystis sp. PCC 6803 with a nucleomorph-encoded open reading frame from the cryptophyte Guillardia theta. BMC Plant Biol. 8:1-12.
Chang L. Liu X, Li Y. Liu CC. Yang F, Zhao J. et al. (2015) Structural organization of an intact phycobilisome and its association with photosystem II. Cell Res. 25 (6): 726-737.
Chaves JE. Melis A (2018) Biotechnology of cyanobacterial isoprene production. Appl Microbiol Biotechnol 102(15):6451-6458
Chaves JE, Rueda Romero P, Kirst H, Melis A (2016) Role of isopentenyl-diphosphate isomerase in heterologous cyanobacterial (Synechocystis) isoprene production. Photosynth Res 130:517-527. DOI: 10.1007/s11120-016-0293-3
Chaves JE, Rueda-Romero P, Kirst H, Melis A (2017) Engineering isoprene synthase expression and activity in cyanobacteria. ACS Synth Biol 6:2281-2292
Cociorva D. D LT, Yates JR (2007) Validation of tandem mass spectrometry database search results using DTASelect. Current protocols in bioinformatics/editoral board, Andreas D Baxevanis [et al] Chapter 13: Unit 13 14.
Collier JL, Grossman AR (1994) A small polypeptide triggers complete degradation of light-harvesting phycobiliproteins in nutrient-deprived cyanobacteria. EMBO J 13:1039-1047
Coragliotti, A. T., Beligni, M. V., Franklin, S. E., and Mayfield, S. P. (2011). Molecular factors affecting the accumulation of recombinant proteins in the Chlamydomonas reinhardtii chloroplast. Mol. Biotechnol. 48, 60-75.
David L. Marx A, Adir N (2011) High-resolution crystal structures of trimeric and rod phycocyanin J Mol Biol 405(1)201-213.
Davies FK, Work VH, Beliaev AS, Posewitz MC (2014) Engineering limonene and bisabolene production in wild type and a glycogen-deficient mutant of Synechococcus sp. PCC 7002. Front. Bioeng. Biotechnol 2:21
De Lorimier R, Bryant DA, Stevens Jr. SE (1990) Genetic analysis of a 9 kDa phycocyanin-associated linker polypeptide. Biochim et Biophys Acta (BBA) Bioenergetics 1019:29-41
De Marsac NT, Cohen-Bazire (1977) Molecular composition of cyanobacterial phycobilisomes. PNAS 74(4):1635-1639.
Demain, A. L., and Vaishna, P. (2009). Production of recombinant proteins by microbes and higher organisms. Biotechnol. Adv. 27, 297-306. doi: 10.1016/j.biotechadv.2009.01.008
Dyo. Y M, and Purton, S. (2018). The algal chloroplast as a synthetic biology platform for production of therapeutic proteins. Microbiology 164, 113-121.
Eaton-Rye, J.J. (2011). Construction of gene interruptions and gene deletions in the cyanobacterium Synechocystis sp. Strain PCC 6803. In: Carpentier R. (eds) Photosynthesis Research Protocols. Methods in Molecular Biology (Methods and Protocols), vol 684. Humana Press, Totowa,. Vol. 684.
Elmorjani K. Herdman M (1987) Metabolic control of phycocyanin degradation in the cyanobacterium Synechocystis pec 6803: a glucose effect. Journal of General Microbiology 133:1685-1694.
Englund E, Liang FY, Pia Lindberg P (2016) Evaluation of promoters and ribosome binding sites for biotechnological applications in the unicellular cyanobacterium Synechocystis sp. PCC 6803. Scientific Reports 6:36640
Formighieri C, Melis A (2014) Regulation of β-phellandrene synthase gene expression, recombinant protein accumulation, and monoterpene hydrocarbons production in Synechocystis transformants. Planta 240:309-324
Formighieri C, Melis A (2014) Regulation of β-phellandrene synthase gene expression. recombinant protein accumulation, and monoterpene hydrocarbons production in Synechocystis transformants. Planta 240:309-324
Formighicri C, Melis A (2015) A phycocyanine⋅phellandrene synthase fusion enhances recombinant protein expression and β-phellandrene (monoterpene) hydrocarbons production in Synechocystis (cyanobacteria). Metab Eng 32:116-124.
Formighieri C, Melis A (2016) Sustainable heterologous production of terpene hydrocarbons in cyanobacteria. Photosynth Res 130.123-135.
Formighieri C, Melis A (2017) Heterologous synthesis of geranyllinalool, a diterpenol plant product, in the cyanobacterium Synechocystis. Appl Microbiol Biotechnol 101:2791-2800 Formighicri C, Melis A (2018) Cyanobacterial production of plant essential oils Planta 248(4):933-946
Gómez-Lojero C. Perez-Gómez B, Shen G, Schluchter WM. Bryant DA (2003) Interaction of ferredoxin: NADP⁺ oxidoreductase with phycobilisomes and phycobilisome substructures of the cyanobacterium Synechococcus sp. strain PCC 7002. Biochemistry. 42(47):13800-13811
Gregory, J. A., Topol, A. B., Doerner, D. Z., Mayfield. S. (2013). Alga-produced cholera toxin-Pfs25 fusion proteins as oral vaccines. Appl Environ Microbiol 79, 3917-3925.
Grossman AR, Bhaya D, Apt KE, Kehoe DM (1995) Light-harvesting complexes in oxygenic photosynthesis: Diversity. Control, and Evolution. Annu. Rev. Genetics 29:231-288
Guan XG, Qin S, Zhao FQ. Zhang XW, Tang XX (2007) Phycobilisomes linker family in cyanobacterial genomes: divergence and evolution. Int. J. Biol. Sci. 3(7):434-445.
Jones. C. S., and Mayfield, S. P. (2013). Steps toward a globally available malaria vaccine: harnessing the potential of algae for future low-cost vaccines. Bioengineered 4, 164-167
Kirst H, Formighieri C, Melis A (2014) Maximizing photosynthetic efficiency and culture productivity in cyanobacteria upon minimizing the phycobilisome light-harvesting antenna size. Biochim Biophys Acta-Bioenergetics 1837(10):1653-1664
Kondo, K., Geng. X., Katayama, M., Ikeuchi, M. (2005). Distinct roles of CpcG1 and CpcG2 in phycobilisome assembly in the cyanobacterium Synechocystis sp PCC 6803. Photosynth Res. 84, 269-273.
Kondo, K., Mullineaux, C.W., Ikeuchi, M (2009) Distinct roles of CpcG1-phycobilisome and CpcG2-phycobilisome in state transitions in a cyanobacterium Synechocystis sp. PCC 6803. Photosynth Res 99:217-225 (2009).
Kondo. K., Ochiai Y, Katayama, M., Ikeuchi, M. (2007). The Membrane-associated CpcG2-phycobilisome in Synechocystis: A new photosystem I antenna. Plant Physiol 144(2):1200-1210.
Li Y, Lin Y, Garvey CJ, Birch D. Corkery RW. Loughlin PC, Scheer H, Willows RD. Chen M (2016) Characterization of red-shifted phycobilisomes isolated from the chlorophyll f-containing cyanobacterium Halomicronema hongdechloris. Biochim Biophys Acta. 1857, 107-114
Lindberg P. Park S. Melis A (2010) Engineering a platform for photosynthetic isoprene production in cyanobacteria, using Synechocystis as the model organism. Metabol Engin 12:70-79
Liu LN, Chen XL, Zhang YZ. Zhou BC (2005) Characterization, structure and function of linker polypeptides in phycobilisomes of cyanobacteria and red algae: An overview. Biochim Biophys Acta 1708:133-142.
McDonald WH, Tabb DL, Sadygov RG, MacCoss MJ, Venable J, et al. (2004) MS1, MS2, and SQT-three unified, compact, and easily parsed file formats for the storage of shotgun proteomic spectra and identifications. Rapid communications in mass spectrometry. RCM 18:2162-2168.
Melis A (1989) Spectroscopic methods in photosynthesis: photosystem stoichiometry and chlorophyll antenna size, Philos. Trans. R. Soc. Lond. B 323:397-409.
Melis A and Duysens LNM (1979) Biphasic energy conversion kinetics and absorbance difference spectra of photosystem II of chloroplasts. Evidence for two different system II reaction centers. Photochem. Photobiol. 29:373-382.
Park, S.K. and Venable, J.D., et. al. (2008) A quantitative analysis software tool for mass spectrometry-based proteomics. Nature methods 5, 319-322
Peng J, Elias JE, Thoreen CC, Licklider LJ. Gygi SP (2003) Evaluation of multidi-mensional chromatography coupled with tandem mass spectrometry (LC/LC-MS/MS) for large-scale protein analysis: the yeast proteome. Journal of proteome research 2; 43-50.
Rasala, B. A., and Mayfield. S. P. (2015). Photosynthetic biomanufacturing in green algae; production of recombinant proteins for industrial, nutritional, and medical uses. Photosynth Res. 123, 227-239.
Richaud C, Zabulon G. Joder A, Thomas JC (2001) Nitrogen or Sulfur Starvation Differentially Affects Phycobilisome Degradation and Expression of the nb1A Gene in Synechocystis Strain PCC 6803. J Bacteriology 183 (10): 2989-2994
Sidler W.A. (1994) Phycobilisome and phycobiliprotein structures. In: Bryant D.A. (eds) The Molecular Biology of Cyanobacteria. Advances in Photosynthesis, Vol 1. Springer, Dordrecht.
Surzycki. R., Greenham, K., Kitayama, K., Dibal, F., Wagner, R., Rochaix, J.-D., et al (2009) Factors effecting expression of vaccines in microalgae. Biologicals 37, 133-138.
Tabb DL, McDonald WH, Yates JR 3rd (2002) DTASelect and Contrast: tools for assembling and comparing protein identifications from shotgun proteomics. Journal of proteome research 1:21-26.
Tran, M., Zhou, B., Pettersson, P. L., Gonzalez, M. J., and Mayfield, S. P. (2009) Synthesis and assembly of a full-length human monoclonal antibody in algal chloroplasts. Biotechnol. Bioeng. 104, 663-673. doi: 10.1002/bit.22446
Ughy B, Ajlani G (2004). Phycobilisome rod mutants in Synechocystis sp. strain PCC6803. Microbiology 150(12):4147-4156.
Van Thor JJ, Gruters OWM, Matthijs HCP, Hellingwerf KJ (1999) Localization and function of ferredoxin: NADP⁺ reductase bound to the phycobilisomes of Synechocystis. EMBO J. 18(15):4128-4136
Watanabe M, Ikeuchi M (2013) Phycobilisome: architecture of a light-harvesting supercomplex. Photosynth Res 116:265-276.
Williams, J.G.K. (1988). Construction of specific mutations in Photosystem II photosynthetic reaction center by genetic engineering methods in Synechocystis 6803. Methods Enzymol. 167, 766-778.
Xu T. et al. ProLuCID, a fast and sensitive tandem mass spectra-based protein identification program. Mol Cell Proteomics. 2006;5(10):S174-S174.
Yamanaka G, Daniel J. Lundell DJ, Glazer AN (1982) Molecular architecture of a light-harvesting antenna. Isolation and characterization of phycobilisome subassembly particles. J Biol Chem 257(8):4077-4086
Yamanaka G. Glazer AN. Williams RC (1978) Cyanobacterial phycobilisomes. Characterization of the phycobilisomes of Synechococcus sp. 6301. J Biol Chem 253 (22): 8303-8310
Zhang XN, Betterle N, Hidalgo Martinez D, Melis A (2021) Recombinant protein stability in cyanobacteria. ACS Synth Biol. 10(4):810-825
Zhou J, Zhang HF, Meng HK, Zhu Y, Bao GH, Zhang YP, Li Y, Ma YH (2014) Discovery of a super-strong promoter enables efficient production of heterologous proteins in cyanobacteria. Scientific Reports 4:4500.

TABLE 1
Mass spectrometry sequencing hits of the unknown ~27 kD
protein band excised from SDS-PAGE lanes. The phycobilisome
peripheral rod-core cylinder linker polypeptide CpcG scored
the highest probability with a 60.6% sequence coverage.

| | | | | | |
|---|---|---|---|---|---|
| | | | | | UniProt |
| Sequence | Spectrum | Sequence | | MW, | accession |
| Count | Count | Coverage (%) | Length | Dalton | number |

Phycobilisome peripheral rod-core cylinder linker polypeptide CpcG

| | | | | | |
|---|---|---|---|---|---|
| 31 | 414 | 60.6 | 249 | 28902 | P73093 |

Photosystem I-associated linker protein CpcL

| | | | | | |
|---|---|---|---|---|---|
| 10 | 15 | 30.5 | 249 | 28523 | P74625 |

C-phycocyanin β-subunit

| | | | | | |
|---|---|---|---|---|---|
| 7 | 18 | 27.9 | 172 | 18126 | Q5471 |

Formyltetrahydrofolate deformylase

| | | | | | |
|---|---|---|---|---|---|
| 2 | 8 | 4.9 | 284 | 32634 | Q55135 |

TABLE 2
Uniprot accession numbers of heterohexameric fusion constructs (α, β*P)₃CpcG, from transformant Synechocystis sp.
PCC 6803, differentially eluted from cobalt column chromatography. Note the presence of the CpcC1 and CpcD linker proteins.

| | | Uniprot accession number. Synechocystis sp. PCC 6803 | | | | | | | |
|---|---|---|---|---|---|---|---|---|---|
| | Strain | P23349 | P26527 | P52981 | P73093 | P73202 | P73203 | P74227 | Q01951 |
| Peptide | Cpc* | 2 | 7 | 35 | 31 | 3 | 0 | 17 | 10 |
| count | Cpc*IFN | 2 | 8 | 24 | 35 | 10 | 9 | 12 | 8 |
| | Cpc*TTFC | 3 | 3 | 7 | 10 | 0 | 0 | 7 | 3 |
| NSAF | Cpc* | 0.6 | 0.5 | 1.9 | 6.4 | 0.9 | 0 | 2.0 | 2.9 |
| (×10⁻²) | Cpc*IFN | 0.2 | 0.4 | 1.2 | 5.0 | 3.3 | 0.7 | 0.9 | 1.2 |
| | Cpc*TTFC | 2.6 | 0.7 | 1.3 | 4.7 | 0.0 | 0 | 1.9 | 2.1 |
| EMPAI | Cpc* | 0.57 | 0.89 | 1.94 | 5.30 | 2.68 | 0 | 1.82 | 2.94 |
| | Cpc*IFN | 0.78 | 1.07 | 1.31 | 5.35 | 5.07 | 1.75 | 1.39 | 2.84 |
| | Cpc*TTFC | 1.25 | 0.21 | 0.32 | 1.13 | 0 | 0.70 | 0.60 | 0 |
| Spectral | Cpc* | 3 | 10 | 57 | 62 | 3 | 0 | 31 | 18 |
| count | Cpc*IFN | 2 | 15 | 67 | 90 | 20 | 14 | 26 | 14 |
| | Cpc*TTFC | 4 | 4 | 12 | 14 | 0 | 0 | 9 | 4 |
| Sequence | Cpc* | 19.5 | 27.7 | 46.9 | 79.9 | 56.6 | 0 | 45.1 | 59.6 |
| coverage | Cpc*IFN | 25 | 31.7 | 36.4 | 80.3 | 78.3 | 44 | 37.8 | 58.4 |
| (%) | Cpc*TTFC | 35.2 | 8.3 | 12.1 | 32.9 | 0 | 0 | 23.1 | 20.5 |

| | | | | | | | | |
|---|---|---|---|---|---|---|---|---|
| Description | 50S | ATP | 1,4- | Phycobilisome | Phycobilisome | Phycobilisome | Elongation | Allophycocyanin |
| | ribosomal | synthase | alpha- | rod-core linker | 8.9 kD linker | 32.1 kD linker | factor Tu | alpha chain |
| | protein | subunit | glucan | polypeptide | polypeptide, | polypeptide, | | ApcA |
| | L7/L12 | beta | branching | CpcG | phycocyanin- | phycocyanin- | | |
| | | | enzyme | | associated, | associated, | | |
| | | | GlgB | | rod CpcD | rod 1 CpcC1 | | |

| | | Uniprot accession number. Synechocystis sp. PCC 6803 | | | | | | |
|---|---|---|---|---|---|---|---|---|
| | Strain | Q01952 | Q02907 | Q02925 | Q05972 | 054714 | Q54715 | Q55318 |
| Peptide | Cpc* | 9 | 7 | 0 | 9 | 29 | 36 | 65 |
| count | Cpc*IFN | 8 | 11 | 2 | 14 | 28 | 32 | 52 |
| | Cpc*TTFC | 0 | 0 | 0 | 2 | 11 | 12 | 23 |
| NSAF | Cpc* | 1.9 | 0.2 | 0 | 0.5 | 17.7 | 25.7 | 10.5 |
| (×10⁻²) | Cpc*IFN | 0.8 | 0.2 | 0.6 | 0.5 | 19.3 | 42.1 | 5.9 |
| | Cpc*TTFC | 0 | 0 | 0 | 0.3 | 28.5 | 26.6 | 22.1 |
| EMPAI | Cpc* | 4.04 | 0.47 | 0 | 0.54 | 5.97 | 8.31 | 6.60 |
| | Cpc*IFN | 3.82 | 0.69 | 0.86 | 1.15 | 7.39 | 9.00 | 4.64 |
| | Cpc*TTFC | 0 | 0 | 0 | 0.13 | 2.86 | 5.34 | 2.37 |
| Spectral | Cpc* | 12 | 7 | 0 | 11 | 118 | 161 | 167 |
| count | Cpc*IFN | 9 | 12 | 3 | 18 | 241 | 495 | 178 |
| | Cpc*TTFC | 0 | 0 | 0 | 2 | 59 | 52 | 110 |
| Sequence | Cpc* | 70.2 | 16.7 | 0 | 18.7 | 84.3 | 96.9 | 88.1 |
| coverage | Cpc*IFN | 68.3 | 22.7 | 26.9 | 33.3 | 92.4 | 100 | 75.1 |
| (%) | Cpc*TTFC | 0 | 0 | 0 | 5.2 | 58.7 | 80.2 | 52.8 |

| | | | | | | | |
|---|---|---|---|---|---|---|---|
| Description | Allophycocyanin | Phycobiliprotein | Phycobilisome | 60 kDa | C-phycocyanin | C-phycocyanin | Ferredoxin- |
| | beta chain ApcB | ApcE | 7.8 kDa linker | chaperonin 1 | beta subunit | beta subunit | NADP reductase |
| | | | polypeptide, | | CpcB | CpcA | |
| | | | allophycocyanin- | | | | |
| | | | associated, core | | | | |
| | | | ApcC | | | | |

TABLE 3
Chlorophyll (Chl) fluorescence yield and rate constants of PSII light absorption and
utilization of Synechocystis transformants upon 620 nm PC actinic excitation. The [Chl]
column shows the chlorophyll concentration loaded in the 1.5 mm pathlength
spectrophotometer cuvette. The Chl a fluorescence yield was measured at 700 nm
in μV of photomultiplier signal output. (In parentheses), fluorescence yield
measurements were normalized to the same [Chl] concentration and reported relative to
that of the Δcpc (=1.00) sample. Rates of light absorption by PSII were measured
from the rate constant k_{II} of the fluorescence induction kinetics of intact
cells suspended in the presence of either 12 or 24 μM DCMU. k_{II}-1 and k_{II}-2
for samples in the presence of 24 μM DCMU show the rate constants obtained upon
a first illumination of dark-adapted cells (k_{II}-1), followed by a 2-min dark
relaxation of the redox state of PSII and upon a second illumination and fluorescence
kinetic registration of the same sample (k_{II}-2). The repeat measurement
was undertaken to test for sample and/or signal deterioration in the presence of DCMU.

| | | Fluorescence | 12 μM | 24 μM | 24 μM |
|---|---|---|---|---|---|
| | [Chl], | yield, μV signal, | DCMU, | DCMU, | DCMU, |
| Strain | μg mL⁻¹ | (normalized) | k_{II}, s⁻¹ | k_{II}-1, s⁻¹ | k_{II}-2 s⁻¹ |
| Δcpc | 34.0 ± 0.0 | 0.0758 ± 0.002 | 8.4 ± 0.2 | 8.4 ± 0.3 | 8.2 ± 0.6 |
| | | (1.00) | | | |
| CpcB*IFN | 42.0 ± 4.2 | 0.1725 ± 0.010 | 11.9 ± 0.5 | 12.1 ± 1.6 | 12.6 ± 0.9 |
| | | (1.84) | | | |
| CpcB*TTFC | 32.5 ± 3.5 | 0.1201 ± 0.005 | 12.2 ± 0.7 | 11.2 ± 0.4 | 11.4 ± 0.4 |
| | | (1.66) | | | |
| CpcB* | 35.5 ± 0.7 | 0.1375 ± 0.009 | 13.0 ± 1.7 | 12.5 ± 1.5 | 13.7 ± 2.1 |
| | | (1.74) | | | |

TABLE 4
Rate constants of PSII light absorption and utilization and Chlorophyll (Chl)
fluorescence yield of Synechocystis transformants upon 620 nm actinic excitation.
The [Chl] column shows the chlorophyll concentration loaded in the 1.5 mm
pathlength spectrophotometer cuvette. Rates of light absorption by PSII in
the Δcpc, CpcA*IFN transformants, and the wild type were measured upon
619.5 nm actinic excitation with 10 μmol photons m⁻² s⁻¹
intensity from the rate constant k_{II} of the fluorescence induction kinetics
of intact cells suspended in the presence of 12 μM DCMU. Numbers in parentheses
show the contribution of phycocyanin to the measured kᵢᵢ (minus the k_{II} of
Δcpc). The Chl a fluorescence yield was measured at 700 nm in V of
photomultiplier signal output. Fluorescence yield measurements were
normalized to the same [Chl] concentration and (in parentheses)
are reported relative to that of the Δcpc (=1.00) sample.

| | | | k_{II}, s⁻¹ | | Fluorescence |
|---|---|---|---|---|---|
| | | | (minus | | yield, V |
| | [Chl], | | the k_{II} | Fluorescence | (normalized to Chl |
| Strain | μg mL−1 | k_{II}, s⁻¹ | of Δcpc) | yield, V | loaded) |
| Δcpc | 23.82± | 6.60 ± 0.21 | 0.00 | 0.0392 ± 0.0010 | 0.0392 |
| | | | | | (1.00) |
| CpcA*IFN-AVE | 27.32 ± 4.02 | 7.33 ± 0.25 | 0.73 (4.1% | 0.0705 ± 0.0038 | 0.0624 |
| | | | of WT) | | (1.59) |
| Wild type | 25.11± | 24.4 ± 0.09 | 17.8 | 0.1408 ± 0.0001 | 0.1336 |
| | | | | | (3.41) |

TABLE 5
Mass spectrometry sequencing analysis of protein bands excised from the 25-37 kDa
SDS-PAGE electrophoretic migration gel section, probing specifically for the
presence of the CpcC1 and CpcC2 linker proteins. SDS-PAGE was loaded with the
same amount of total protein extract from the strains denoted as WT, CpcG1*IFN
and double fusion CpcB*IFN + CpcG1*IFN. The qualitative test consistently showed
presence of the phycocyanin CpcC1-32 kDa linker polypeptide in all strains examined,
and absence of the CpcC2-30 kDa linker polypeptide in either of the two transformants.
Both CpcC1 and CpcC2 were detected in extracts of the wild type. The CpcC1 linker
functions in the structural stability of the middle (α, β)₃(α, β)₃ phycocyanin
disc, while CpcC2 linker is associated with the stability of the distal
(α, β)₃(α, β)₃ disc in the phycocyanin rod structure.

| | | | | Sequence | Length, | Mol | | |
|---|---|---|---|---|---|---|---|---|
| | Protein | Sequence | Spectrum | Coverage | amino | Wt | NSAF | EMPAI |
| Strain | Probed | Count | Count | (%) | acids | (kDa) | (×10⁻³) | (×10⁻³) |
| WT | CpcC1 | 1 | 1 | 8 | 291 | 32.5 | 9.9 | 207.8 |
| | CpcC2 | 2 | 2 | 17 | 273 | 30.8 | 21.0 | 462.2 |
| CpcG1*IFN | CpcC1 | 4 | 6 | 20 | 291 | 32.5 | 52.6 | 595.9 |
| | CpcC2 | nd | nd | nd | nd | nd | nd | nd |
| CpcB*TTFC + | CpcC1 | 3 | 3 | 15 | 291 | 32.5 | 48.2 | 406.0 |
| CpcG1*IFN | CpcC2 | nd | nd | nd | nd | nd | nd | nd |

Variables measured:
Sequence count shows the number of unique parent ions identified for a specific protein.
Spectrum count shows the total number of spectra identified for a specific protein.
Sequence coverage is total coverage of amino acids for a protein sequence by identified peptides
Length represents the number of amino acids of an identified protein
Mol Wt: molecular weight
NSAF: Normalized Spectral Abundance Factor
emPAI: Exponentially modified Protein Abundance Index
Descriptive name of all proteins identified in the excised 25-37 kDa bands.
Wild type #1: Phycobilisome 32.5 kDa linker polypeptide, phycocyanin-associated, rod 1 OS = Synechocystis sp. GN = cpcC1 PE = 1 SV = 3
Wild type #2: Phycobilisome 30.8 kDa linker polypeptide, phycocyanin-associated, rod 2 OS = Synechocystis sp. GN = cpcC2 PE = 1 SV = 1
CpcG1*IFN: Phycobilisome 32.5 kDa linker polypeptide, phycocyanin-associated, rod 1 OS = Synechocystis sp. GN = cpcC1 PE = 1 SV = 3
CpcB*TTFC + CpcG1*IFN #2: Phycobilisome 32.5 kDa linker polypeptide, phycocyanin-associated, rod 1 OS = Synechocystis sp. OX = 1111708 GN = cpcC1 PE = 1 SV = 3

TABLE 6
Rate constants of PSII light absorption and utilization and chlorophyll
(Chl) fluorescence yield of Synechocystis transformants upon 620 nm
actinic excitation at 10 μmol photons m⁻² s⁻¹ intensity.
The [Chl] column shows the chlorophyll concentration loaded in the
1.5 mm pathlength spectrophotometer cuvette. Rates of light absorption
by PSII in the Δcpc, two transformants used in the technical section,
and the wild type were measured from the rate constant k_{II}
of the fluorescence induction kinetics of intact cells suspended in the
presence of 12 μM DCMU. Numbers in parentheses show the
contribution of phycocyanin to the measured kᵢᵢ (minus
the k_{II} of Δcpc). The Chl a fluorescence yield was measured
at 700 nm in V of photomultiplier signal output. Fluorescence
yield measurements were normalized to the same [Chl] concentration
and (in parentheses) are reported relative to that of the Δcpc (=1.00) sample.

| | | | | | Fluorescence |
|---|---|---|---|---|---|
| | | | k_{II}, s⁻¹ | | yield, V |
| | [Chl], | | (minus the | Fluorescence | normalized |
| Strain | μg mL⁻¹ | k_{II}, s⁻¹ | k_{II} of Δcpc) | yield, V | to Chl |
| Δcpc | 24.11 ± 3.94 | 7.87 ± 0.66 | 0.00 | 0.0759 ± 0.01 | 0.0759 |
| | | | | | (1.0) |
| CpcG1*IFN | 28.43 ± 1.61 | 11.99 ± 1.13 | 4.12 ± 0.19 | 0.1384 ± 0.01 | 0.1174 |
| | | | (22.4% of | | (1.55) |
| | | | WT) | | |
| CpcB*TTFC & | 22.74 ± 5.58 | 10.34 ± 0.73 | 2.47 ± 0.06 | 0.1039 ± 0.02 | 0.1102 |
| CpcG1*IFN | | | (13.4% of | | (1.45) |
| | | | WT) | | |
| Wild type | 26.02 ± 3.19 | 26.24 ± 2.63 | 18.37 ± 1.97 | 0.2046 ± 0.02β | 0.1896 |
| | | | | | (2.50) |

Illustrative Expression Constructs and Sequences

I. CpcB*IFN construc sequences, designed for insertion in the cpc operon
(see, FIG. 1B)
DNA sequence
Lowercase, cpcB gene
Lowercase italics, His-tag
Lowercase bold, tev cleavage site
Uppercase bold, codon-optimized human interferon (IFN) gene + stop codon
Lowercase bold italics, BglII DNA restriction site + intergenic seq
Uppercase italics, cmR (chloramphenicol) gene for antibiotic selection
Lowercase underline, transcription terminator + BamHI DNA restriction site
Uppercase, cpcB-cpcA intergenic sequence
Uppercase bold italics, partial cpcA gene
atgttcgacgtattcactcgggttgtttcccaagctgatgctcgcggcgagtacctctctggttctcagttagatgctttgagcgctaccgtt
gctgaaggcaacaaacggattgattctgttaaccgcatcaccggtaatgcttccgctatcgtttccaacgctgctcgtgctttgttcgccg
aacagccccaattaatccaacccggtggaaacgcctacaccagccgtcgtatggctgcttgtttgcgtgacatggaaatcatcctccgc
tatgttacctacgcaaccttcaccggcgacgcttccgttctagaagatcgttgcttgaacggtctccgtgaaacctacgttgccctgggtg
ttcccggtgcttccgtagctgctggcgttcaaaaaatgaaagaagctgccctggacatcgttaacgatcccaatggcatcacccgtggt
gattgcagtgctatcgttgctgaaatcgctggttacttcgaccgcgccgctgctgccgtagcccaccatcaccatcaccatgataattt
gtatttacaaggcTGTGACTTGCCTCAGACGCATTCTTTGGGAAGCCGACGCACACT
GATGCTGCTCGCCCAAATGCGCCGGATCTCCTTATTCTCCTGTCTCAAGGAT
CGGCATGACTTCGGCTTCCCTCAGGAGGAGTTTGGAAATCAGTTCCAAAAG
GCCGAAACCATTCCGGTCCTCCATGAAATGATTCAACAGATCTTTAACTTAT
TCAGTACCAAAGACAGCAGTGCGGCCTGGGACGAAACATTACTCGATAAAT
TCTACACGGAATTATACCAACAGTTGAACGACTTAGAAGCCTGTGTAATCCA
AGGTGTTGGTGTCACTGAGACTCCATTAATGAAAGAAGACTCTATTCTGGCC
GTCCGCAAGTATTTCCAGCGAATCACACTGTATTTGAAAGAGAAAAAGTATT
CTCCGTGTGCGTGGGAGGTAGTACGGGCTGAAATCATGCGGTCCTTCTCTTT
AAGCACAAACCTCCAGGAATCTCTGCGCTCCAAAGAATGAagatctgcggccgcgttgatcgg
cacgtaagaggttccaactttcaccataatgaaataagatcactaccgggcgtattttttgagttatcgagattttcaggagctaaggaagctaa
aATGGAGAAAAAAATCACTGGATATACCACCGTTGATATATCCCAATGGCATCGTAAAGAACATTTTGAGGCATTTCAG
TCAGTTGCTCAATGTACCTATAACCAGACCGTTCAGCTGGATATTACGGCCTTTTTAAAGACCGTAAAGAA
AAATAAGCACAAGTTTTATCCGGCCTTTATTCACATTCTTGCCCGCCTGATGAATGCTCATCCGGAATTCC
GTATGGCAATGAAAGACGGTGAGCTGGTGATATGGGATAGTGTTCACCCTTGTTACACCGTTTTCCATGA
GCAAACTGAAACGTTTTCATCGCTCTGGAGTGAATACCACGACGATTTCCGGCAGTTTCTACACATATATT
CGCAAGATGTGGCGTGTTACGGTGAAAACCTGGCCTATTTCCCTAAAGGGTTTATTGAGAATATGTTTTTC
GTCTCAGCCAATCCCTGGGTGAGTTTCACCAGTTTTGATTTAAACGTGGCCAATATGGACAACTTCTTCGC
CCCCGTTTTCACCATGGGCAAATATTATACGCAAGGCGACAAGGTGCTGATGCCGCTGGCGATTCAGGTT
CATCATGCCGTCTGTGATGGCTTCCATGTCGGCAGAATGCTTAATGAATTACAACAGTACTGCGATGAGTG
GCAGGGGGGGGCGTAAtttttttaaggcagttattggtgcccttaaacgcctggggatccTCTGGTTATTTTAAAAACCAACTTT
ACTCAGGTTCCATACCCGAGAAAATCCAGCTTAAAGCTGACATATCTAGGAAAATTTTCACATTCT
AACGGGAGATACCAGAACAATGAAAACCCCTTTAACTGAAGCCGTTTCCACCGCTGACTCTCAAGG
TCGCTTTCTGAGCAGCACCGAATTGCAAATTGCTTTCGGTCGTCTACGTCAAGCTAATGCTGGTTTGC
AAGCCGCTAAAGCTCTGACCGACAATGCCCAGAGCTTGGTAAATGGTGCTGCCCAAGCCGTTTATAA
CAAATTCCCCTACACCACCCAAACCCAAGGCAACAACTTTGCTGCGGATCAACGGGGTAAAGACAAG
TGTGCCCGGGACATCGGCTACTACCTCCGCATCGTTACCTACTGCTTAGTTGCTGGTGGTACCGGTC
CTTTGGATGAGTACTTGATCGCCGGTATTGATGAAATCAACCGCACCTTTGACCTCTCCCCCAGCTGG
TATGTT
Amino acid sequence encoded by the CpcB*IFN construct
Lowercase, CpcB protein
Lowercase italics, His-tag
Lowercase bold, TEV cleavage site
Uppercase bold, human interferon a-2 (IFN) protein
mfdvftrvvsqadargeylsgsqldalsatvaegnkridsvnritgnasaivsnaaralfaeqpqliqpggnaytsrrmaaclrdme
iilryvtyatftgdasvledrcinglretyvalgvpgasvaagvqkmkeaaldivndpngitrgdcsaivaciagyfdraaaavxhh
hhhhenlyfqgCDLPQTHSLGSRRTLMLLAQMRRISLFSCLKDRHDFGFPQEEFGNQ
FQKAETIPVLHEMIQQIFNLFSTKDSSAAWDETLLDKFYTELYQQLNDLEACVIQ
GVGVTETPLMKEDSILAVRKYFqRITLYLKEKKYSPCAWEVVRAEIMRSFSLST
NLQ
II. CpcB*TTFC construct sequences, designed for insertion in the cpc operon (see FIG. 1C)
DNA sequences
Lowercase, cpcB gene
Uppercase, S7 spacer
Lowercase italics, His-tag
Lowercase bold, TEV cleavage site
Uppercase bold, Tetanus toxin Fragment C (TTFC) + stop codon
Lowercase bold italics, RBS (from Formighieri and Melis 2016)
Uppercase italics, smR gene for antibiotic selection
Lowercase underline, transcription terminator + intergenic seq + partial cpcA gene
atgttcgacgtattcactcgggttgtttcccaagctgatgctcgcggcgagtacctctctggttctcagttagatgctttgagcgctaccgtt
gctgaaggcaacaaacggattgattctgttaaccgcatcaccggtaatgcttccgctatcgtttccaacgctgctcgtgctttgttcgccg
aacagccccaattaatccaacccggtggaaacgcctacaccagccgtcgtatggctgcttgtttgcgtgacatggaaatcatcctccgc
tatgttacctacgcaaccttcaccggcgacgcttccgttctagaagatcgttgcttgaacggtctccgtgaaacctacgttgccctgggtg
ttcccggtgcttccgtagctgctggcgttcaaaaaatgaaagaagctgccctggacatcgttaacgatcccaatggcatcacccgtggt
gattgcagtgctatcgttgctgaaatcgctggttacttcgaccgcgccgctgctgccgtagccCCCATGCCTTGGCGCGT
GATTcaccatcaccatcaccatgataatttgtatttacaaggcAAGAACTTAGACTGTTGGGTCGATA
ATGAGGAGGATATCGATGTCATTCTAAAGAAGTCTACCATCCTAAATCTGGA
CATTAACAATGATATCATTAGTGATATTTCTGGTTTTAATTCTTCTGTTATCA
CATACCCCGACGCCCAATTAGTTCCAGGAATTAATGGGAAGGCTATTCATCT
AGTAAATAATGAGAGCAGCGAAGTGATCGTCCACAAGGCGATGGACATTGA
GTATAATGATATGTTCAACAACTTTACTGTGTCCTTTTGGTTGCGCGTCCCC
AAAGTGTCTGCCAGTCACCTGGAACAATACGACACGAATGAATATAGTATCA
TTAGCAGTATGAAAAAGTATAGTTTAAGTATTGGGTCTGGGTGGTCCGTCTC
TCTCAAAGGAAACAACCTCATCTGGACCCTCAAGGATTCTGCAGGCGAAGT
GCGTCAAATTACATTCCGCGACTTGTCCGATAAATTCAATGCGTACCTCGCT
AACAAATGGGTTTTCATCACCATCACGAACGACCGGCTGAGTAGCGCTAACC
TCTACATTAATGGCGTGTTGATGGGGAGTGCGGAGATCACCGGCCTGGGGG
CAATTCGCGAGGACAACAACATCACACTCAAGTTGGACCGTTGCAATAACAA
CAACCAATATGTCTCTATCGACAAATTTCGTATTTTCTGTAAGGCGCTAAAC
CCAAAGGAGATCGAAAAGTTATATACTAGTTATTTGAGCATCACGTTTTTAC
GCGATTTTTGGGGCAACCCACTGCGTTATGACACTGAATATTATCTCATTCC
CGTTGCGTACAGCAGTAAAGACGTCCAATTAAAGAATATCACGGATTATATG
TATCTGACTAATGCTCCCAGTTACACGAACGGGAAATTAAACATTTACTACC
GCCGTCTGTACTCTGGTCTGAAGTTTATTATCAAACGCTACACCCCCAACAA
TGAAATCGACTCTTTTGTTCGGTCTGGTGACTTTATTAAACTGTACGTAAGT
TACAACAACAATGAACACATCGTGGGATACCCTAAAGACGGGAATGCGTTC
AATAACTTAGATCGGATCCTCCGAGTAGGGTATAATGCACCCGGTATTCCTC
TGTATAAGAAGATGGAAGCGGTAAAGCTCCGTGACCTCAAAACTTATAGCGT
GCAACTCAAACTGTACGACGACAAAGATGCGTCTCTAGGGTTGGTGGGTAC
CCACAACGGACAAATCGGGAATGACCCTAACCGCGATATTCTAATCGCTTCT
AATTGGTATTTTAACCACTTAAAAGATAAGACCCTCACCTGCGACTGGTATT
TCGTCCCAACCGACGAGGGATGGACTAATGATTGAggaattaggaggtaatatATGAGG
GAAGCGGTGATCGCCGAAGTATCGACTCAACTATCAGAGGTAGTTGGCGTCATCGAGC
GCCATCTCGAACCGACGTTGCTGGCCGTACATTTGTACGGCTCCGCAGTGGATGGCG
GCCTGAAGCCACACAGTGATATTGATTTGCTGGTTACGGTGACCGTAAGGCTTGATGA
AACAACGCGGCGAGCTTTGATCAACGACCTTTTGGAAACTTCGGCTTCCCCTGGAGAG
AGCGAGATTCTCCGCGCTGTAGAAGTCACCATTGTTGTGCACGACGACATCATTCCGT
GGCGTTATCCAGCTAAGCGCGAACTGCAATTTGGAGAATGGCAGCGCAATGACATTCT
TGCAGGTATCTTCGAGCCAGCCACGATCGACATTGATCTGGCTATCTTGCTGACAAAAG
CAAGAGAACATAGCGTTGCCTTGGTAGGTCCAGCGGCGGAGGAACTCTTTGATCCGGT
TCCTGAACAGGATCTATTTGAGGCGCTAAATGAAACCTTAACGCTATGGAACTCGCCGC
CCGACTGGGCTGGCGATGAGCGAAATGTAGTGCTTACGTTGTCCCGCATTTGGTACAG
CGCAGTAACCGGCAAAATCGCGCCGAAGGATGTCGCTGCCGACTGGGCAATGGAGCG
CCTGCCGGCCCAGTATCAGCCCGTCATACTTGAAGCTAGACAGGCTTATCTTGGACAA
GAAGAAGATCGCTTGGCCTCGCGCGCAGATCAGTTGGAAGAATTTGTCCACTACGTGA
AAGGCGAGATCACCAAGGTAGTCGGCAAATAAtttttttaaggcagttattggtgcccttaaacgcctggggat
cctctggttattttaaaaaccaactttactcagcttccatacccgagaaaatccagcttaaagctgacatatctaggaaaattttcacattcta
acgggagataccagaacaatgaaaacccctttaactgaagccgtttccaccgctgactctcaaggtcgctttctgagcagcaccgaatt
gcaaattgctttcggtcgtctacgtcaagctaatgctggtttgcaagccgctaaagctctgaccgacaatgcccagagcttggtaaatgg
tgctgcccaagccgtttataacaaattcccctacaccacccaaacccaaggcaacaactttgctgcggatcaacggggtaaagacaag
tctgccccggacatcgcctactacctccgcatccttacctactccttagttcctggtgctacccctcctttcgatgagtacttgatccccc
gtattga
Amino acid sequence encoded by the CpcB*TTFC construct
Lowercase, CpcB protein
Uppercase, S7 spacer
Lowercase italics, His-tag
Lowercase bold, TEV cleavage site
Uppercase bold, Tetanus toxin Fragment C (TTFC) + stop codon
mfdvftrvvsqadargeylsgsqldalsatvaegnkridsvnritgnasaivsnaaralfaeqpqliqpggnaytsrrmaaclrdm
elilryvtyatftgdasvledrclnglretyvalgvpgasvaagvqkmkeaaldivndpngitrgdcsaivaeiagyfdraaaavxP
MPWRVIhhhhhhenlyfqgKNLDCWVDNEEDIDVILKKSTILNLDINNDIISDISGENSSVITYPDAQLVPG
INGKAIHLVNNESSEVIVHKAMDIEYNDMENNFTVSFWLRVPKVSASHLEQYDTNEYSIISSMKKYSLSI
GSGWSVSLKGNNLIWTLKDSAGEVRQITFRDLSDKFNAYLANKWVFITITNDRLSSANLYINGVLMGS
AEITGLGAIREDNNITLKLQRCNNNNQYVSIDKFRIFCKALNPKEIEKLYTSYLSITFLRDFWGNPLRYDTE
YYLIPVAYSSKDVQLKNITDYMYLTNAPSYTNGKLNIYYRRLYSGLKFIIKRYTPNNEIDSFVRSGDFIKLY
VSYNNNEHIVGYPKDGNAFNNLDRILRVGYNAPGIPLYKKMEAVKLRDLKTYSVQLKLYDDKDASLGL
VGTHNGQIGNDPNRDILIASNWYFNHLKDKTLTCQWYFVPTDEGWTND*
III. CpcB* construct sequences, designed for insertion in the cpc operon (see, FIG. 1D)
DNA sequences:
Lowercase, cpcB gene
Uppercase, S7 spacer
Lowercase italics, His-tag
Lowercase bold, tev cleavage site
Uppercase bold, stop codon
Lowercase bold italics, RBS (from Formighieri and Melis 2016)
Uppercase italics, smR gene for antibiotic selection
Lowercase underline, transcription terminator + intergenic seq + partial cpcA gene
atgttcgacgtattcactcgggttgtttcccaagctgatgctcgcggcgagtacctctctggttctcagttagatgctttgagcgctaccgtt
gctgaaggcaacaaacggattgattctgttaaccgcatcaccggtaatgcttccgctatcgtttccaacgctgctcgtgctttgttcgccg
aacagccccaattaatccaacccggtggaaacgcctacaccagccgtcgtatggctgcttgtttgcgtgacatggaaatcatcctccgc
tatgttacctacgcaaccttcaccggcgacgcttccgttctagaagatcgttgcttgaacggtctccgtgaaacctacgttgccctgggtg
ttcccggtgcttccgtagctgctggcgttcaaaaaatgaaagaagctgccctggacatcgttaacgatcccaatggcatcacccgtggt
gattgcagtgctatcgttgctgaaatcgctggttacttcgaccgcgccgctgctgccgtagccCCCATGCCTTGGCGCGT
GATTcaccatcaccatcaccatgataatttgtatttacaaggcTGAggaattaggaggtaatatATGAGGGAAGCG
GTGATCGCCGAAGTATCGACTCAACTATCAGAGGTAGTTGGCGTCATCGAGCGCCATC
TCGAACCGACGTTGCTGGCCGTACATTTGTACGGCTCCGCAGTGGATGGCGGCCTGA
AGCCACACAGTGATATTGATTTGCTGGTTACGGTGACCGTAAGGCTTGATGAAACAACG
CGGCGAGCTTTGATCAACGACCTTTTGGAAACTTCGGCTTCCCCTGGAGAGAGCGAGA
TTCTCCGCGCTGTAGAAGTCACCATTGTTGTGCACGACGACATCATTCCGTGGCGTTAT
CCAGCTAAGCGCGAACTGCAATTTGGAGAATGGCAGCGCAATGACATTCTTGCAGGTA
TCTTCGAGCCAGCCACGATCGACATTGATCTGGCTATCTTGCTGACAAAAGCAAGAGAA
CATAGCGTTGCCTTGGTAGGTCCAGCGGCGGAGGAACTCTTTGATCCGGTTCCTGAAC
AGGATCTATTTGAGGCGCTAAATGAAACCTTAACGCTATGGAACTCGCCGCCCGACTG
GGCTGGCGATGAGCGAAATGTAGTGCTTACGTTGTCCCGCATTTGGTACAGCGCAGTA
ACCGGCAAAATCGCGCCGAAGGATGTCGCTGCCGACTGGGCAATGGAGCGCCTGCC
GGCCCAGTATCAGCCCGTCATACTTGAAGCTAGACAGGCTTATCTTGGACAAGAAGAA
GATCGCTTGGCCTCGCGCGCAGATCAGTTGGAAGAATTTGTCCACTACGTGAAAGGCG
AGATCACCAAGGTAGTCGGCAAATAAtttttttaaggcagttattggtgcccttaaacgcctggggatcctctggttat
tttaaaaaccaactttactcagcttccatacccgagaaaatccagcttaaagctcacatatctaggaaaattttcacattctaacgggagat
accagaacaatgaaaacccctttaactgaagccgtttccaccgctgactctcaaggtcgctttctgagcagcaccgaattgcaaattgct
ttcggtcgtctacgtcaagctaatcctgctttgcaagcccctaaagctctgaccgacaatgcccagagcttggtaaatggtgctgcccaa
gccgtttataacaaattcccctacaccacccaaacccaaggcaacaactttcctccggatcaacggggtaaagacaagtctccccgg
gacatcggctactacctccgcatcgttacctactgcttagttcctggtggtaccggtcctttcgatgagtacttgatcgccggtattga
Amino acid sequence encoded by the CpcB* construct
Lowercase, CpcB protein
Uppercase, S7 spacer
Lowercase italics, His-tag
Lowercase bold, TEV cleavage site
mfdvflrvvsqadargeylsgsqldalsatvaegnkridsvnritgnasaivsnaaralfaeqpqliqpggnaytsrmmaaclrdme
iilryvtyatftgdasvledrclngretyvalgvpgasvaagvqkmkeaaldivndpngitrgdesaivaciagyfdraaaavxP
MPWRVIhhhhhhenlyfqg*
IV. CpcA*IFN construct cpcA-S7-6xHis-tev-IFN-CmR-cpcC2 sequences (see, FIG. 13B)
DNA sequences
lowercase cpcA no STOP codon
UPPERCASE S7
lower case italics His6x, histidine tag (6x)
UPPERCASE ITALICS tev, TEV protease cleavage site
UPPERCASE BOLD IFN, codon-optimized human interferon gene for expression in
Synechocystis
lower case bold intergenic sequence
UPPERCASE underline cmR (chloramphenicol selection cassette)
lowercase underline intergenic sequence
lowercase italics bold cpcC2 (partial) for homologous recombination
atgaaaacccctttaactgaagccgtttccaccgctgactctcaaggtcgctttctgagcagcaccgaattgcaaattgctttcggtcgtct
acgtcaagctaatgctggtttgcaagccgctaaagctctgaccgacaatgcccagagcttggtaaatggtgctgcccaagccgtttata
acaaattcccctacaccacccaaacccaaggcaacaactttgctgcggatcaacggggtaaagacaagtgtgcccgggacatcggct
actacctccgcatcgttacctactgcttagttgctggtggtaccggtcctttggatgagtacttgatcgccggtattgatgaaatcaaccgc
acctttgacctctcccccagctggtatgttgaagctctgaaatacatcaaagctaaccacggcttgagtggcgatgcccgtgacgaagc
taattcctacctcgattacgccatcaatgctctgagcCCCATGCCTTGGCGCGTGATTcaccatcaccatcaccatG
AAAATTTGTATTTACAAGGCTGTGACTTGCCTCAGACGCATTCTTTGGGAAGCCG
ACGCACACTGATGCTGCTCGCCCAAATGCGCCGGATCTCCTTATTCTCCTGT
CTCAAGGATCGGCATGACTTCGGCTTCCCTCAGGAGGAGTTTGGAAATCAG
TTCCAAAAGGCCGAAACCATTCCGGTCCTCCATGAAATGATTCAACAGATCT
TTAACTTATTCAGTACCAAAGACAGCAGTGCGGCCTGGGACGAAACATTACT
CGATAAATTCTACACGGAATTATACCAACAGTTGAACGACTTAGAAGCCTGT
GTAATCCAAGGTGTTGGTGTCACTGAGACTCCATTAATGAAAGAAGACTCTA
TTCTGGCCGTCCGCAAGTATTTCCAGCGAATCACACTGTATTTGAAAGAGAA
AAAGTATTCTCCGTGTGCGTGGGAGGTAGTACGGGCTGAAATCATGCGGTC
CTTCTCTTTAAGCACAAACCTCCAGGAATCTCTGCGCTCCAAAGAATGAgcggc
cgcgttgatcggcacgtaagaggttccaactttcaccataatgaaataagatcactaccgggcgtattttttgagttatcgagat
tttcaggagctaaggaagctaaaATGGAGAAAAAAATCACTGGATATACCACCGTTGATATA
TCCCAATGGCATCGTAAAGAACATTTTGAGGCATTTCAGTCAGTTGCTCAATGTA
CCTATAACCAGACCGTTCAGCTGGATATTACGGCCTTTTTAAAGACCGTAAAGAA
AAATAAGCACAAGTTTTATCCGGCCTTTATTCACATTCTTGCCCGCCTGATGAAT
GCTCATCCGGAATTCCGTATGGCAATGAAAGACGGTGAGCTGGTGATATGGGAT
AGTGTTCACCCTTGTTACACCGTTTTCCATGAGCAAACTGAAACGTTTTCATCGCT
CTGGAGTGAATACCACGACGATTTCCGGCAGTTTCTACACATATATTCGCAAGAT
GTGGCGTGTTACGGTGAAAACCTGGCCTATTTCCCTAAAGGGTTTATTGAGAATA
TGTTTTTCGTCTCAGCCAATCCCTGGGTGAGTTTCACCAGTTTTGATTTAAACGTG
GCCAATATGGACAACTTCTTCGCCCCCGTTTTCACCATGGGCAAATATTATACGC
AAGGCGACAAGGTGCTGATGCCGCTGGCGATTCAGGTTCATCATGCCGTCTGTGA
TGGCTTCCATGTCGGCAGAATGCTTAATGAATTACAACAGTACTGCGATGAGTGG
CAGGGGGGGGCGTAAtcagtttttaattctagctggcctggaagggtgggaaagtgttaacaactggttgacaatattccg
cttttccaggtcttgtcgtatttattgacacttatctaggagaacaaaatgactagtttagtttcggcccagcgtctgggcattgtggccgtg
gatgaggctattcccctcgagcttcgttcccggagtacagaggaagaggtggatgccgttatcctggcggtttaccgtcaagttttgg
gcaacgatcatctcatgtcccagguacgactguccagtgcagaatctttgctccggggcagggagatttcggtaagggattttgttc
gggctgtggctctgtcggaagtgtaccggcagaagtttttccattccaacccacaaaatcgttttatcgagcttaattataagcattta
ctgggacgggctccctacgatcagtcggaaattgctttccacaccgatctttatcaccaggggggctat
Amino Acid Sequences encoded by construct
CpcA =
MKTPLTEAVSTADSQGRFLSSTELQIAFGRLRQANAGLQAAKALTDNAQSLVNGAA
QAVYNKFPYTTQTQGNNFAADQRGKDKCARDIGYYLRIVTYCLVAGGTGPLDEYLI
AGIDEINRTFDLSPSWYVEALKYIKANHGLSGDARDEANSYLDYAINALS
S7 = PMPWRVI
6xHis = HHHHHH
tev = ENLYLQG
IFN =
CDLPQTHSLGSRRTLMLLAQMRRISLFSCLKDRHDFGFPQEEFGNQFQKAETIPVLHE
MIQQIFNLFSTKDSSAAWDETLLDKFYTELYQQLNDLEACVIQGVGVTETPLMKEDSI
LAVRKYFQRITLYLKEKKYSPCAWEVVRAEIMRSFSLSTNLQESLRSKE*
Intergenic sequence = AAALIGT*EVPTFTIMK*DHYRAYFLSYRDFQELRKL
CmR =
MEKKITGYTTVDISQWHRKEHFEAFQSVAQCTYNQTVQLDITAFLKTVKKNKHKFY
PAFIHILARLMNAHPEFRMAMKDGELVIWDSVHPCYTVFHEQTETFSSLWSEYHDDF
RQFLHIYSQDVACYGENLAYFPKGFIENMFFVSANPWVSFTSFDLNVANMDNFFAPV
FTMGKYYTQGDKVLMPLAIQVHHAVCDGFHVGRMLNELQQYCDEWQGGA*
Intergenic sequence= SVFNSSWPGRVGKC*QLVDNIPLFQVLSYLLTLI*ENK
CpcC2=
MTSLVSAQRLGIVAVDEAIPLELRSRSTEEEVDAVILAVYRQVLGNDHLMSQERLTS
AESLLRGREISVRDFVRAVALSEVYRQKFFHSNPQNRFIELNYKHLLGRAPYDQSEIA
FHTDLYHQGGY
V. CpcG1*IFN construct cpcg1-S7-6xHis-tev IFN-CMR-cpcG1 3′ sequences (see, FIG. 17B)
DNA sequences
lowercase cpcG1 no STOP codon
UPPER CASE S7
lower case italics His6x, histidine tag (6x)
UPPER CASE ITALICS tev, TEV protease cleavage site
UPPER CASE BOLD IFN, codon-optimized human interferon gene for expression in
Synechocystis
lower case bold
intergenic sequence
UPPER CASE underline cmR (chloramphenicol selection cassette)
lowercase italics bold cpcG1 downstream for homologous recombination
atggctcttcccctattgaactacgcccccaaaagtcaaaatgtgagggtagaaggttatgaaattggctctgaagagaagcctgttgttt
tcaccacggaaaacatcctctccagcagcgatatggataacctaatcgaggcggcctatcgtcaaatctttttccatgcgtttaagtggg
accgagaaaaagtccttgagtcccaactgcgtaacggccaaattactgtacgagattttgtgcggggcttgttgctttccaacaccttccg
caatagcttctacgaaaagaatagtaactaccgcttcgttgagcactgtgtacagaagattttaggggggacgtttacagcgaacggg
aaaaaattgcttggtccattgtggtcgcgaccaagggctatcaaggattaattgacgatttgctcaacagcgacgagtacctcaataactt
tggctatgacacggtgccctaccaacgtcgtcgcaaccttcccggtcgggaagcgggtgaattgccctttaacatcaaatctccccggt
acgatgcctaccaccgtcgccaattgggcttcccccaaatcgtttggcagaacgaagtgcgtcgctttattccccaggagaaaaagctc
accgctggcaatccgatgaacttcttgggcatggcccgcagtatcaaccctgccgccaacaccattcccaaggtttccgcccaaaatat
caacatcgaagcttctgtgccccgtcgcCCCATGCCTTGGCGCGTGATTcaccatcaccatcaccatGAAAAT
TTGTATTTACAAGGCTGTGACTTGCCTCAGACGCATTCTTTGGGAAGCCGACGC
ACACTGATGCTGCTCGCCCAAATGCGCCGGATCTCCTTATTCTCCTGTCTCA
AGGATCGGCATGACTTCGGCTTCCCTCAGGAGGAGTTTGGAAATCAGTTCC
AAAAGGCCGAAACCATTCCGGTCCTCCATGAAATGATTCAACAGATCTTTAA
CTTATTCAGTACCAAAGACAGCAGTGCGGCCTGGGACGAAACATTACTCGAT
AAATTCTACACGGAATTATACCAACAGTTGAACGACTTAGAAGCCTGTGTAA
TCCAAGGTGTTGGTGTCACTGAGACTCCATTAATGAAAGAAGACTCTATTCT
GGCCGTCCGCAAGTATTTCCAGCGAATCACACTGTATTTGAAAGAGAAAAAG
TATTCTCCGTGTGCGTGGGAGGTAGTACGGGCTGAAATCATGCGGTCCTTCT
CTTTAAGCACAAACCTCCAGGAATCTCTGCGCTCCAAAGAATGAgcggccgcgttga
tcggcacgtaagaggttccaactttcaccataatgaaataagatcactaccgggcgtattttttgagttatcgagattttcagga
gctaaggaagctaaaATGGAGAAAAAAATCACTGGATATACCACCGTTGATATATCCCA
ATGGCATCGTAAAGAACATTTTGAGGCATTTCAGTCAGTTGCTCAATGTACCTAT
AACCAGACCGTTCAGCTGGATATTACGGCCTTTTTAAAGACCGTAAAGAAAAAT
AAGCACAAGTTTTATCCGGCCTTTATTCACATTCTTGCCCGCCTGATGAATGCTCA
TCCGGAATTCCGTATGGCAATGAAAGACGGTGAGCTGGTGATATGGGATAGTGT
TCACCCTTGTTACACCGTTTTCCATGAGCAAACTGAAACGTTTTCATCGCTCTGGA
GTGAATACCACGACGATTTCCGGCAGTTTCTACACATATATTCGCAAGATGTGGC
GTGTTACGGTGAAAACCTGGCCTATTTCCCTAAAGGGTTTATTGAGAATATGTTT
TTCGTCTCAGCCAATCCCTGGGTGAGTTTCACCAGTTTTGATTTAAACGTGGCCA
ATATGGACAACTTCTTCGCCCCCGTTTTCACCATGGGCAAATATTATACGCAAGG
CGACAAGGTGCTGATGCCGCTGGCGATTCAGGTTCATCATGCCGTCTGTGATGGC
TTCCATGTCGGCAGAATGCTTAATGAATTACAACAGTACTGCGATGAGTGGCAGG
GCGGGGCGTAAgcactaaggtcagagggttgaaggattagtgcattcctagcgtggatagttactgatttttccgtcctga
aaattaaactgaaaaatcaaataattgtttcgctgggactgtttgctagtcccttttttttggctatttttggggcgaggatctgagtataa
atacttgattgaactatgacgggagagcaatttagcggaaattgtgaagtcttctaacaattatggaacctctgcccataatcccag
actaaaaacctatacttgcccccaataccaactcctcaccgaacctagggcttgatgttggttgaggtagtaatttccccagagtgg
agtccccatgattgcccctatttgccgtgctctgcgttcccgtcttcccctcgccgctatgctgatgattgccgccactgctacccccgc
cttagcgaattcccccctcgccgccaatttaactccgaatcaaccccaacctttactgttggatggcgttcaac
Amino acid sequences encoded by construct
CpcG1 =
MALPLLNYAPKSQNVRVEGYEIGSEEKPVVFTTENILSSSDMDNLIEAAYRQIFFHAF
KWDREKVLESQLRNGQITVRDFVRGLLLSNTFRNSFYEKNSNYRFVEHCVQKILGRD
VYSEREKIAWSIVVATKGYQGLIDDLLNSDEYLNNFGYDTVPYQRRRNLPGREAGEL
PFNIKSPRYDAYHRRQLGFPQIVWQNEVRRFIPQEKKLTAGNPMNFLGMARSINPAA
NTIPKVSAQNINIEASVPRR
Invalidate
S7 = PMPWRVI
6xHis = HHHHHH
tev = ENLYLQG
IFN =
CDLPQTHSLGSRRTLMLLAQMRRISLFSCLKDRHDFGFPQEEFGNQFQKAETIPVLHE
MIQQIFNLFSTKDSSAAWDETLLDKFYTELYQQLNDLEACVIQGVGVTETPLMKEDSI
LAVRKYFQRITLYLKEKKYSPCAWEVVRAEIMRSFSLSTNLQESLRSKE*
Intergenic sequence = AAALIGT*EVPTFTIMK*DHYRAYFLSYRDFQELRKL
CmR =
MEKKITGYTTVDISQWHRKEHFEAFQSVAQCTYNQTVQLDITAFLKTVKKNKHKFY
PAFIHILARLMNAHPEFRMAMKDGELVIWDSVHPCYTVFHEQTETFSSLWSEYHDDF
RQFLHIYSQDVACYGENLAYFPKGFIENMFFVSANPWVSFTSFDLNVANMDNFFAPV
FTMGKYYTQGDKVLMPLAIQVHHAVCDGFHVGRMLNELQQYCDEWQGGA*
CpcG1 3′ =
ALRSEG*RISAFLAWIVTDFSVLKIKLKNQIIVSLGLFASPFFLAIFGARI*V*ILD*TMT
GEQFSGNCEVF*QLWNLCP*SQTKNLYLPPIPTPHRT*GLMLVEVVISPEWSPHDCPY
LPCSAFPSSPRRYADDCRHCYPRLSEFPPRRQFNSESTPTFTVGWR
VI. IFN*CpcA construct cpcB-CmR-IFN-tev-6xHis-S7-cpcA sequences (see, FIG. 27A)
DNA sequences
lowercase cpcB, native B-subunit of phycocyanin gene
lowercase italics native intergenic sequence between cpcB and cpcA
UPPERCASE cmR (chloramphenicol selection cassette)
lowercase bold native intergenic sequence between cpcB and cpcA
UPPERCASE BOLD IFN, codon-optimized human interferon gene with ATG and no
stop codon
lowercase underline tev, TEV protease cleavage site
UPPERCASE underline His₆ₓ, histidine tag (6x)
UPPERCASE ITALICS Spacer S7
lowercase bold underline CpcA without ATG
atgttcgacgtattcactcgggttgtttcccaagctgatgctcgcggcgagtacctctctggttctcagttagatgctttgagcgctaccgtt
gctgaaggcaacaaacggattgattctgttaaccgcatcaccggtaatgcttccgctatcgtttccaacgctgctcgtgctttgttcgccg
aacagccccaattaatccaacccggtggaaacgcctacaccagccgtcgtatggctgcttgtttgcgtgacatggaaatcatcctccgc
tatgttacctacgcaaccttcaccggcgacgcttccgttctagaagatcgttgcttgaacggtctccgtgaaacctacgttgccctgggtg
ttcccggtgcttccgtagctgctggcgttcaaaaaatgaaagaagctgccctggacatcgttaacgatcccaatggcatcacccgtggt
gattgcagtgctatcgttgctgaaatcgctggttacttcgaccgcgccgctgctgccgtagcctagtctggttattttaaaaaccaacttt
actcaggttccatacccgagaaaatccagcttaaagctgacatatctaggaaaattttcacattctaacgggagataccagaaca
ATGGAGAAAAAAATCACTGGATATACCACCGTTGATATATCCCAATGGCATCGT
AAAGAACATTTTGAGGCATTTCAGTCAGTTGCTCAATGTACCTATAACCAGACCG
TTCAGCTGGATATTACGGCCTTTTTAAAGACCGTAAAGAAAAATAAGCACAAGTT
TTATCCGGCCTTTATTCACATTCTTGCCCGCCTGATGAATGCTCATCCGGAATTCC
GTATGGCAATGAAAGACGGTGAGCTGGTGATATGGGATAGTGTTCACCCTTGTTA
CACCGTTTTCCATGAGCAAACTGAAACGTTTTCATCGCTCTGGAGTGAATACCAC
GACGATTTCCGGCAGTTTCTACACATATATTCGCAAGATGTGGCGTGTTACGGTG
AAAACCTGGCCTATTTCCCTAAAGGGTTTATTGAGAATATGTTTTTCGTCTCAGCC
AATCCCTGGGTGAGTTTCACCAGTTTTGATTTAAACGTGGCCAATATGGACAACT
TCTTCGCCCCCGTTTTCACCATGGGCAAATATTATACGCAAGGCGACAAGGTGCT
GATGCCGCTGGCGATTCAGGTTCATCATGCCGTCTGTGATGGCTTCCATGTCGGC
AGAATGCTTAATGAATTACAACAGTACTGCGATGAGTGGCAGGGGGGGGCGTAA
ggttattttaaaaaccaactttactcaggttccatacccgagaaaatccagcttaaagctgacatatctaggaaaattttcacat
tctaacgggagataccagaacaATGTGTGACTTGCCTCAGACGCATTCTTTGGGAAGCC
GACGCACACTGATGCTGCTCGCCCAAATGCGCCGGATCTCCTTATTCTCCTG
TCTCAAGGATCGGCATGACTTCGGCTTCCCTCAGGAGGAGTTTGGAAATCA
GTTCCAAAAGGCCGAAACCATTCCGGTCCTCCATGAAATGATTCAACAGATC
TTTAACTTATTCAGTACCAAAGACAGCAGTGCGGCCTGGGACGAAACATTAC
TCGATAAATTCTACACGGAATTATACCAACAGTTGAACGACTTAGAAGCCTG
TGTAATCCAAGGTGTTGGTGTCACTGAGACTCCATTAATGAAAGAAGACTCT
ATTCTGGCCGTCCGCAAGTATTTCCAGCGAATCACACTGTATTTGAAAGAGA
AAAAGTATTCTCCGTGTGCGTGGGAGGTAGTACGGGCTGAAATCATGCGGT
CCTTCTCTTTAAGCACAAACCTCCAGGAATCTCTGCGCTCCAAAGAAgataatttct
atttacaaggcCACCATCACCATCACCATCCCATGCCTTGGCGCGtGATTaaaacccctttaactg
aagccgtttccacccctgactctcaagctcgctttctgagcagcaccgaattccaaattcctttccctcctctacctcaacctaat
gctggtttgcaagccgctaaagctctgaccgacaatgcccagagcttcgtaaatggtgctgcccaagccgtttataacaaattc
ccctacaccacccaaacccaaggcaacaactttcctcccgatcaacgcgctaaagacaagtctgccccggacatcgcctact
acctccgcatccttacctactccttagttcctggtcgtaccggtcctttcgatgagtacttgatcgccgctattgatgaaatcaac
cgcacctttgacctctcccccagctggtatcttgaagctctgaaatacatcaaagctaaccaccgcttcagtggcgatgcccgt
gacgaagctaattcctacctcgattacgccatcaatgctctgagctag
Amino acid sequences:
CpcB =
MFDVFTRVVSQADARGEYLSGSQLDALSATVAEGNKRIDSVNRITGNASAIVSNAAR
ALFAEQPQLIQPGGNAYTSRRMAACLRDMEIILRYVTYATFTGDASVLEDRCLNGLR
ETYVALGVPGASVAAGVQKMKEAALDIVNDPNGITRGDCSAIVAEIAGYFDRAAAA
VA*
Native intergenic sequence between cpcB and cpcA =
SGYFKNQLYSGSIPEKIQLKADISRKIFTF*REIPE
CmR =
MEKKITGYTTVDISQWHRKEHFEAFQSVAQCTYNQTVQLDITAFLKTVKKNKHKFY
PAFIHILARLMNAHPEFRMAMKDGELVIWDSVHPCYTVFHEQTETFSSLWSEYHDDF
RQFLHIYSQDVACYGENLAYFPKGFIENMFFVSANPWVSFTSFDLNVANMDNFFAPV
FTMGKYYTQGDKVLMPLAIQVHHAVCDGFHVGRMLNELQQYCDEWQGGA*
Native intergenic sequence between cpcß and cpcA =
GYFKNQLYSGSIPEKIQLKADISRKIFTF*REIPE
IFN =
MCDLPQTHSLGSRRTLMLLAQMRRISLFSCLKDRHDFGFPQEEFGNQFQKAETIPVL
HEMIQQIFNLFSTKDSSAAWDETLLDKFYTELYQQLNDLEACVIQGVGVTETPLMKE
DSILAVRKYFQRITLYLKEKKYSPCAWEVVRAEIMRSFSLSTNLQESLRSKE
tev = ENLYLQG
6xHis = HHHHHH
S7 = PMPWRVI
CpcA =
KTPLTEAVSTADSQGRFLSSTELQIAFGRLRQANAGLQAAKALTDNAQSLVNGAAQ
AVYNKFPYTTQTQGNNFAADQRGKDKCARDIGYYLRIVTYCLVAGGTGPLDEYLIA
GIDEINRTFDLSPSWYVEALKYIKANHGLSGDARDEANSYLDYAINALS*
VII. IFN*CpcG1 construct cpcG1 5′-CmR-IFN-tev-6xHis-S7-cpcGI sequences (see, FIG. 27B)
DNA sequences
UPPERCASE cpcG1 5′
UPPERCASE underline cmR (chloramphenicol selection cassette)
lowercase native intergenic sequence between cpcB and cpcA
UPPERCASE BOLD IFN, codon-optimized human interferon gene with ATG and no
stop codon
lowercase italics tev, TEV protease cleavage site
lowercase bold His₆ₓ, histidine tag (6x)
UPPERCASE italics Spacer S7
lowercase underline cpcG1 without ATG
CCCTCATTCCCGATGAAGACAATCCCCAACAGGGACTACTACTGCGGGAGAGGG
GCTATGCGGAAATTGTCCCCATTGCCGGTCGCTATCACATCGACGAAGAGGAAG
CCCTGGTGCTAGTGACGGAGTATGAGACCATGACCACCATTGAACGGTTCTGGTT
TGCCAACCCGGATATGCGCCTTAGAACCAGTACTGTGCAAAGGTTTGGGGGATTC
AATACCGCCACCTACTGTACGGAAATGCGGGTCAAAGAGGAAAACACCGTGTCT
GCGTCCCCTGCCCCTGCCTACGAACAATTTTGTGGCTGGTAGCCCCGTTTGGTTCC
AGTATGGGGAGCTCGATCGCCCTGCCAAGCCATCTGAGGAATTGGCTAACCCTG
AATGGAAGGGCGATAATTGTAATAAATTGTAATATTTGCCAACGATTAGGACCCT
TCCCCCTTATTATTTAAACTAACTAATTATCAAGCTCTTCTTTAAGATCACGGAGG
TTTACACATGGAGAAAAAAATCACTGGATATACCACCGTTGATATATCCCAATGG
CATCGTAAAGAACATTTTGAGGCATTTCAGTCAGTTGCTTCAATGTACCTATAACC
AGACCGTTCAGCTGGATATTACGGCCTTTTTAAAGACCGTAAAGAAAAATAAGC
ACAAGTTTTATCCGGCCTTTATTCACATTCTTGCCCGCCTGATGAATGCTCATCCG
GAATTCCGTATGGCAATGAAAGACGGTGAGCTGGTGATATGGGATAGTGTTCAC
CCTTGTTACACCGTTTTCCATGAGCAAACTGAAACGTTTTCATCGCTCTGGAGTG
AATACCACGACGATTTCCGGCAGTTTCTACACATATATTCGCAAGATGTGGCGTG
TTACGGTGAAAACCTGGCCTATTTCCCTAAAGGGTTTATTGAGAATATGTTTTTCG
TCTCAGCCAATCCCTGGGTGAGTTTCACCAGTTTTGATTTAAACGTGGCCAATAT
GGACAACTTCTTCGCCCCCGTTTTCACCATGGGCAAATATTATACGCAAGGCGAC
AAGGTGCTGATGCCGCTGGCGATTCAGGTTCATCATGCCGTCTGTGATGGCTTCC
ATGTCGGCAGAATGCTTAATGAATTACAACAGTACTGCGATGAGTGGCAGGGCG
GGGCGTAAggttattttaaaaaccaactttactcaggttccatacccgagaaaatccagcttaaagctgacatatctaggaaaattt
tcacattctaacgggagataccagaacaATGTGTGACTTGCCTCAGACGCATTCTTTGGGAAG
CCGACGCACACTGATGCTGCTCGCCCAAATGCGCCGGATCTCCTTATTCTCC
TGTCTCAAGGATCGGCATGACTTCGGCTTCCCTCAGGAGGAGTTTGGAAATC
AGTTCCAAAAGGCCGAAACCATTCCGGTCCTCCATGAAATGATTCAACAGAT
CTTTAACTTATTCAGTACCAAAGACAGCAGTGCGGCCTGGGACGAAACATTA
CTCGATAAATTCTACACGGAATTATACCAACAGTTGAACGACTTAGAAGCCT
GTGTAATCCAAGGTGTTGGTGTCACTGAGACTCCATTAATGAAAGAAGACTC
TATTCTGGCCGTCCGCAAGTATTTCCAGCGAATCACACTGTATTTGAAAGAG
AAAAAGTATTCTCCGTGTGCGTGGGAGGTAGTACGGGCTGAAATCATGCGG
TCCTTCTCTTTAAGCACAAACCTCCAGGAATCTCTGCGCTCCAAAGAAgataattt
gtatttacaaggccaccatcaccatcaccatCCCATGCCTTGGCGCGTGATTgctcttcccctattgaactacgcccc
caaaagtcaaaatgtgagggtagaaggttatgaaattggctctgaagagaagcctgttgttttcaccacggaaaacatcctctccagca
gcgatatggataacctaatcgaggcggcctatcgtcaaatctttttccatgcgtttaagtcggaccgagaaaaagtccttgagtcccaact
gcgtaacggccaaattactgtacgagattttgtgcggggcttgttgctttccaacaccttccgcaatagcttctacgaaaagaatagtaac
taccgcttcgttgagcactgtgtacagaagattttaggggggacgtttacagcgaacgggaaaaaattgcttggtccattgtggtcgcg
accaagggctatcaaggattaattgacgatttgctcaacagcgacgagtacctcaataactttggctatgacacggtgccctaccaacgt
cgtcgcaaccttcccggtcgggaagcgggtgaattgccctttaacatcaaatctccccggtacgatgcctaccaccgtcgccaattggg
cttcccccaaatcctttggcagaacgaagtccgtcgctttattccccaggagaaaaagctcacccctggcaatccgatgaacttcttgg
gcatggcccgcagtatcaaccctgccgccaacaccattcccaaggtttccgcccaaaatatcaacatcgaagcttctctgccccgtcgc
tag
Amino acid sequences encoded by construct
CpcG1 5′ =
PSFPMKTIPNRDYYCGRGAMRKLSPLPVAITSTKRKPWC**RSMRP*PPLNGSGLPTRI
CALEPVLCKGLGDSIPPPTVRKCGSKRKTPCLRPLPLPTNNFVAGSPVWFQYGELDRP
AKPSEELANPEWKGDNCNKL*YLPTIRTLPPYYLN-LIIKLFFKITEVY
CmR =
MEKKITGYTTVDISQWHRKEHFEAFQSVAQCTYNQTVQLDITAFLKTVKKNKHKFY
PAFIHILARLMNAHPEFRMAMKDGELVIWDSVHPCYTVFHEQTETFSSLWSEYHDDF
RQFLHIYSQDVACYGENLAYFPKGFIENMFFVSANPWVSFTSFDLNVANMDNFFAPV
FTMGKYYTQGDKVLMPLAIQVHHAVCDGFHVGRMLNELQQYCDEWQGGA*
Native intergenic sequence between cpcB and cpcA =
GYFKNQLYSGSIPEKIQLKADISRKIFTF*REIPE
IFN =
MCDLPQTHSLGSRRTLMLLAQMRRISLFSCLKDRHDFGFPQEEFGNQFQKAETIPVL
HEMIQQIFNLFSTKDSSAAWDETLLDKFYTELYQQLNDLEACVIQGVGVTETPLMKE
DSILAVRKYFQRITLYLKEKKYSPCAWEVVRAEIMRSFSLSTNLQESLRSKE
tev = ENLYLQG
6xHis = HHHHHH
S7 = PMPWRVI
CpcG1 =
ALPLLNYAPKSQNVRVEGYEIGSEEKPVVFTTENILSSSDMDNLIEAAYRQIFFHAFK
WDREKVLESQLRNGQITVRDFVRGLLLSNTFRNSFYEKNSNYRFVEHCVQKILGRDV
YSEREKIAWSIVVATKGYQGLIDDLLNSDEYLNNFGYDTVPYQRRRNLPGREAGELP
FNIKSPRYDAYHRRQLGFPQIVWQNEVRRFIPQEKKLTAGNPMNFLGMARSINPAAN
TIPKVSAQNINIEASVPRR*

What is claimed is:

1. A method of producing a protein of interest in a cyanobacteria host cell, wherein the protein of interest is encoded by a recombinant expression unit comprising:
(i) a nucleic acid sequence encoding a fusion protein comprising the protein of interest fused at the carboxyl terminus or amino terminus of a cyanobacterial CpcB protein, wherein the fusion protein is expressed as a component of functional (α,β*P)₃CpcG or (α,P*β*)₃CpcG heterohexameric discs, where α is a cyanobacterial CpcA phycocyanin subunit protein, β is a cyanobacterial CpcB phycocyanin subunit protein, asterisk denotes fusion, P is the protein of interest, and CpcG is a phycocyanin linker polypeptide;
(ii) a nucleic acid sequence encoding the CpcA phycocyanin subunit protein; and
(iii) a nucleic acid sequence encoding the CpcG phycocyanin linker polypeptide: and
wherein the method comprises:
(a) culturing the cyanobacterial host cell comprising the recombinant expression unit under conditions in which functional heterohexameric discs comprising the fusion protein are expressed; and
(b) purifying the heterohexameric discs comprising the protein of interest to at least 90% (w/w) purity.

2. The method of claim 1, wherein the recombinant expression unit is operably linked to an endogenous cyanobacteria cpc promoter.

3. The method of claim 1 or 2, wherein the fusion protein comprises a protease cleavage site between CpcB protein and the polypeptide of interest.

4. A recombinant cyanobacterial host cell comprising a (α,β)₃CpcG heterohexameric disc, wherein a is a cyanobacterial CpcA phycocyanin subunit protein, β is a cyanobacterial CpcB phycocyanin subunit protein, and CpcG is a phycocyanin linker polypeptide; and wherein a cyanobacterial protein selected from CpcB, CpcA, and CpcG is fused to a first protein of interest to be expressed in the cyanobacterial host cell and a further different cyanobacterial protein selected from CpcB, CpcA, and CpcG is fused to a second protein of interest to be expressed in the cyanobacterial host cell, wherein the second protein of interest may be the same protein as the first protein of interest or a different protein.

5. The recombinant cyanobacterial host cell of claim 4, wherein a first protein of interest is fused to a CpcB and the second protein of interest is fused to CpcA.

6. The recombinant cyanobacterial host cell of claim 4, wherein a first protein of interest is fused to a CpcB and the second protein of interest is fused to CpcG.

7. The recombinant cyanobacterial host cell of claim 4, wherein a first protein of interest is fused to a CpcA and the second protein of interest is fused to CpcG.

8. A recombinant cyanobacterial host cell comprising a (α,β)₃CpcG heterohexameric disc, wherein α is a cyanobacterial CpcA phycocyanin subunit protein, β is a cyanobacterial CpcB phycocyanin subunit protein, and CpcG is a phycocyanin linker polypeptide, and wherein a protein of interest is fused to the N-terminus or C-terminus of CpcG; or the protein of interest if fused to the N-terminus of CpcB or the N-terminus of CpcA.

9. A recombinant cyanobacterial host cell comprising a first and a second fusion protein, wherein the first and the second fusion protein are encoded by a recombinant expression unit that expresses one or more proteins of interest, wherein the first fusion protein comprises a first protein of interest fused at the carboxyl terminus or amino terminus of a CpcB protein and the second fusion protein comprises a second protein of interest, which may be the same or different from the first protein of interest, fused at the carboxyl terminus or amino terminus of a CpcA protein; and the first and second fusion proteins are expressed as a component of functional (α*P2,β*P1)₃CpcG or (α*P2,P1*β)₃CpcG or (P2*α,β*P1)₃CpcG or (P2*α,P1*β)₃CpcG heterohexameric discs, wherein:
α is a cyanobacterial CpcA phycocyanin subunit protein, β is a cyanobacterial CpcB phycocyanin subunit protein, asterisk denotes fusion, P1 is the first protein of interest, P2 is the second protein of interest, and CpcG is a phycocyanin linker polypeptide.

10. A recombinant cyanobacteria host cell comprising a first and a second fusion protein, wherein the first and the second fusion protein are encoded by a recombinant expression unit that expresses one or more proteins of interest, wherein the first fusion protein comprises a first protein of interest fused at the carboxyl terminus or amino terminus of a CpcB protein and the second fusion protein comprises a second protein of interest, which may be the same or different from the first protein of interest, fused at the carboxyl terminus or amino terminus of a CpcA protein; and the first and second fusion proteins are expressed as a component of functional (α*P2,β*P1)₃CpcC1 or (α*P2,P1*β)₃CpcC1 or (P2*α,β*P1)₃CpcC1 or (P2*α,P1*β)₃CpcC1 heterohexameric discs, wherein:
α is a cyanobacterial CpcA phycocyanin subunit protein, β is a cyanobacterial CpcB phycocyanin subunit protein, asterisk denotes fusion, P1 is the first protein of interest, P2 is the second protein of interest, and CpcC1 is a phycocyanin linker polypeptide.

11. The recombinant cyanobacteria host cell of claim 9 or 10, wherein the first fusion protein comprises a protease cleavage site between CpcB and the first polypeptide of interest and the second fusion protein comprises a cleavage site between CpcA and the second polypeptide of interest.

12. A recombinant cyanobacterial host cell comprising a first and a second fusion protein, wherein the first and the second fusion protein are encoded by a recombinant expression unit that expresses one or more proteins of interest, wherein the first fusion protein comprises a first protein of interest fused at the carboxyl terminus or amino terminus of a CpcG protein and the second fusion protein comprises a second protein of interest, which may be the same or different from the first protein of interest, fused at the carboxyl terminus or amino terminus of a CpcB protein; and the first and second fusion proteins are expressed as a component of functional (α,β*P2)₃CpcG*P1 or (α,P2*β)₃CpcG*P1 or (α,β*P2)₃P1*CpcG or (α,P2*β)₃P1*CpcG heterohexameric discs, wherein:
α is a cyanobacterial CpcA phycocyanin subunit protein, β is a cyanobacterial CpcB phycocyanin subunit protein, asterisk denotes fusion, P1 is the first protein of interest, P2 is the second protein of interest, and CpcG is a phycocyanin linker polypeptide.

13. The recombinant cyanobacteria host cell of claim 12, wherein the first fusion protein comprises a protease cleavage site between CpcG and the first polypeptide of interest and the second fusion protein comprises a cleavage site between CpcB and the second polypeptide of interest.

14. A recombinant cyanobacterial host cell comprising a first and a second fusion protein, wherein the first and the second fusion protein are encoded by a recombinant expression unit that expresses one or more proteins of interest, wherein the first fusion protein comprises a first protein of interest fused at the carboxyl terminus or amino terminus of a CpcG protein and the second fusion protein comprises a second protein of interest, which may be the same or different from the first protein of interest, fused at the carboxyl terminus or amino terminus of a CpcA protein; and the first and second fusion proteins are expressed as a component of functional (α*P2,62 )₃CpcG*P1 or (P2*α,β)₃CpcG*P1 or (α*P2,β)₃P1*CpcG or (α*P2,β)₃P1*CpcG heterohexameric discs, wherein:
α is a cyanobacterial CpcA phycocyanin subunit protein, β is a cyanobacterial CpcB phycocyanin subunit protein, asterisk denotes fusion, P1 is the first protein of interest, P2 is the second protein of interest, and CpcG is a phycocyanin linker polypeptide.

15. The recombinant cyanobacteria host cell of claim 14, wherein the first fusion protein comprises a protease cleavage site between CpcG and the first polypeptide of interest and the second fusion protein comprises a cleavage site between CpcA and the second polypeptide of interest.

16. The recombinant cyanobacteria host cell of any one of claims 4-15, wherein the recombinant expression unit is operably linked to an endogenous cyanobacteria cpc promoter.

17. The recombinant cyanobacteria boost cell of any one of claims 4-15, wherein the cyanobacteria is a single celled cyanobacteria.

18. The recombinant cyanobacteria host cell of claim 17, wherein the cyanobacteria is a Synechococcus sp., a Thermosynechococcus sp., a Synechocystis sp., or a Cyanothece sp.

19. The recombinant cyanobacteria host cell of any one of claims 4-15, wherein the cyanobacteria are micro-colonial cyanobacteria.

20. The recombinant cyanobacteria host cell of claim 19, wherein the cyanobacteria is a Gloeocapsa magma, Gloeocapsa phylum, Gloeocapsa alpicola, Gloeocpasa atrata, Chroococcus spp., or Aphanothece sp.

21. The recombinant cyanobacteria host cell of any one of claims 4-15, wherein the cyanobacteria is a filamentous cyanobacteria.

22. The recombinant cyanobacteria boost cell of claim 21, wherein the cyanobacteria is an Oscillatoria spp., a Nostoc sp., an Anabaena sp., or an Arthrospira sp.

23. The recombinant cyanobacteria host cell of any one of claims 4-22 wherein at least one of the proteins of interest is isoprene synthase, a β-phellandrene synthase, a geranyl diphosphate synthase, a geranyl linalool synthase, human interferon α-2 protein, cholera toxin B (CtxB), or a tetanus toxin fragment C (TTFC); or G-CSF, GM-CSF, MCP1 sCD40L, TGF-alpha, EGF, FGF-2, Flt-3L, INF-apha2, INF-gamma, IL-10, IL-15, IL-17, IL-1beta, IL-2, IL-6, IL-8, IL-10, IL-15, IL-17, IL-1beta, IL-2, IL-6, IL-8, IP-10, MIP-1beta, PDGF-AA, TNF-alpha, or VEGF.

24. A cyanobacteria culture comprising a host cell of any one of claims 4-23.

25. A method of producing a first and a second protein of interest, the method comprising growing a cyanobacteria culture of claim 24 under conditions in which the first and the second protein of interest are expressed.

26. A recombinant cyanobacteria host cell comprising a recombinant expression unit comprising:
(i) a nucleic acid sequence encoding a cyanobacterial CpcA phycocyanin subunit protein,
(ii) a nucleic acid sequence encoding a cyanobacterial CpcB phycocyanin subunit protein, and
(iii) a nucleic acid sequence encoding a fusion protein comprising a protein of interest fused at the carboxyl terminus or amino terminus of a cyanobacterial CpcG polypeptide,
wherein the fusion protein is expressed as a component of functional heterohexameric discs comprising the cyanobacterial CpcA phycocyanin subunit protein, the cyanobacterial CpcB phycocyanin subunit protein, and the fusion protein fused at the carboxyl terminus or amino terminus of the cyanobacterial CpcG polypeptide.

27. The recombinant cyanobacteria host cell of claim 26, wherein the recombinant expression unit is operably linked to an endogenous cyanobacteria cpc promoter.

28. The recombinant cyanobacteria host cell of claim 26 or 27, wherein the fusion protein comprises a protease cleavage site between CpcG and the protein of interest.

29. The recombinant cyanobacteria host cell of any one of claims 26-28, wherein the cyanobacteria is a single celled cyanobacteria.

30. The recombinant cyanobacteria boost cell of claim 29, wherein the cyanobacteria is a Synechococcus sp., a Thermosynechococcus sp., a Synechocystis sp., or a Cyanothece sp.

31. The recombinant cyanobacteria host cell of any one of claims 26-28, wherein the cyanobacteria are micro-colonial cyanobacteria.

32. The recombinant cyanobacteria host cell of claim 31, wherein the cyanobacteria is a Gloeocapsa magma, Gloeocapsa phylum, Gloeocapsa alpicola, Gloeocpasa atrata, Chroococcus spp., or Aphanothece sp.

33. The recombinant cyanobacteria boost cell of any one of claims 26-28, wherein the cyanobacteria is a filamentous cyanobacteria.

34. The recombinant cyanobacteria host cell of claim 33, wherein the cyanobacteria is an Oscillatoria spp., a Nostoc sp., an Anabaena sp., or an Arthrospira sp.

35. The recombinant cyanobacteria host cell of any one of claims 26-34, wherein the protein of interest is isoprene synthase, a β-phellandrene synthase, a geranyl diphosphate synthase, a geranyl linalool synthase, human interferon α-2 protein, cholera toxin B (CtxB), or a tetanus toxin fragment C (TTFC); or G-CSF, GM-CSF, MCPI sCD40L, TGF-alpha, EGF, FGF-2, Flt-3L, INF-apha2, INF-gamma, IL-10, IL-15, IL-17, IL-1beta, IL-2, IL-6, IL-8, IL-10, IL-15, IL-17, IL-1beta, IL-2, IL-6, IL-8, IP-10, MIP-1beta, PDGF-AA, TNF-alpha, or VEGF.

36. A cyanobacteria culture comprising a host cell of any one of claims 26-35.

37. A heterohexameric disc preparation at least 90% pure, comprising heterohexameric discs comprising a cyanobacterial CpcA phycocyanin subunit protein, a cyanobacterial CpcB phycocyanin subunit protein, and CpcG or CpcC1 phycocyanin linker polypeptides, wherein at least one of CpcA, CpB, CpcG, or CpC1 is fused at a C-terminal or N-terminal end to a protein of interest expressed in cyanobacteria.

38. The heterohexameric disc preparation of claim 37, wherein the protein of interest is linked to the C-terminal end of CpcB.
